(19) United States
(12) Patent Application Publication
(10) Pub. No.: US 2025/0213563 A1
(43) Pub. Date: Jul. 3, 2025

(54) SUBSTITUTED PYRIMIDIN-4(3H)-ONES FOR USE IN TREATING CANCER

(71) Applicant: NAVIRE PHARMA, INC., Palo Alto, CA (US)

(72) Inventors: Pedro BELTRAN, Palo Alto, CA (US); Carl DAMBKOWSKI, Palo Alto, CA (US); Justin LIM, Palo Alto, CA (US); Kerstin SINKEVICIUS, Palo Alto, CA (US); Anna WADE, Palo Alto, CA (US); Eli WALLACE, Palo Alto, CA (US); Lauren WOOD, Palo Alto, CA (US); David VAN VEENHUYZEN, Palo Alto, CA (US); Susanna WEN, Palo Alto, CA (US)

(21) Appl. No.: 18/698,694

(22) PCT Filed: Oct. 6, 2022
(86) PCT No.: PCT/US2022/045875

§ 371 Date: Apr. 4, 2024

Related U.S. Application Data
(60) Provisional application No. 63/252,970, filed on Oct. 6, 2021.
(60) Provisional application No. 63/330,529, filed on Apr. 13, 2022.

(51) Int. Cl. A61K 31/513; A61P 35/00

(52) U.S. Cl. CPC A61K 31/513; A61P 35/00

(57) ABSTRACT

The present disclosure provides a method of treating cancer or a solid tumor (e.g., an advanced or metastatic solid tumor) in a subject with a therapeutically effective amount of a compound of formula (I) or a pharmaceutically acceptable salt, hydrate, solvate, stereoisomer, conformational isomer, tautomer, or a combination thereof, where the subject has one or more mutations in the MAPK pathway and/or one or more mutations in PTPN11. In particular, the solid tumor is an advanced or metastatic KRAS mutant solid tumor (e.g., KRAS G12C-positive solid tumor); an advanced or metastatic NF1 loss-of-function (LOF) solid tumor or BRAF class II/III mutant solid tumor; or chordoma.

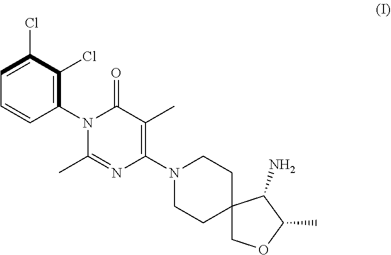

### CROSS-REFERENCES TO RELATED APPLICATIONS

This application claims priority to U.S. Provisional Application No. 63/252,970 filed Oct. 6, 2021 and U.S. Provisional Application No. 63/330,529 filed Apr. 13, 2022, each of which is incorporated herein in its entirety for all purposes.

### STATEMENT AS TO RIGHTS TO INVENTIONS MADE UNDER FEDERALLY SPONSORED RESEARCH AND DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK

Not Applicable

### BACKGROUND

Protein-tyrosine phosphatase non-receptor type 11 (PTPN11, also known as Src Homology-2 phosphatase (SHP2)) is a non-receptor protein tyrosine phosphatase encoded by the PTPN11 gene. This PTP contains two tandem Src homology-2 (SH2) domains, which function as phospho-tyrosine binding domains, a catalytic domain, and a C-terminal tail. In the basal state the protein typically exists in an inactive, self-inhibited conformation with the N-terminal SH2 domain blocking the active site. When stimulated by signal transduction mediated by cytokines and growth factor binding of phosphorylated proteins to the SH2 domains the auto-inhibition is relieved, this makes the active site available for dephosphorylation of PTPN11 substrates (MG Mohl, BG Neel, Curr. Opin. Genetics Dev. 2007, 17, 23-30. KS Grossmann, Adv. Cancer Res. 2010, 106, 53-89. W. Q. Huang et. al. Curr. Cancer Drug Targets 2014, 14, 567-588. C. Gordon et. al. Cancer Metastasis Rev. 2008, 27, 179-192.).

Germ-line and somatic mutations in PTPN11 have been reported in several human diseases resulting in gain-of-function in the catalytic activity, including Noonan Syndrome and Leopard Syndrome; as well as multiple cancers such as juvenile myelomonocytic leukemia, neuroblastoma, myelodysplastic syndrome, B cell acute lymphoblastic leukemia/lymphoma, melanoma, acute myeloid leukemia and cancers of the breast, lung and colon (M G Mohl, B G Neel, Curr. Opin. Genetics Dev. 2007, 17, 23-30). Recent studies have demonstrated that single PTPN11 mutations are able to induce Noonan syndrome, JMML-like myeloproliferative disease and acute leukemia in mice. These mutations disrupt the auto-inhibition between the N—SH2 domains and the catalytic site allowing constitutive access of substrates to the catalytic site of the enzyme (E. Darian et al, Proteins, 2011, 79, 1573-1588. Z-H Yu et al, JBC, 2013, 288, 10472, W Qiu et al BMC Struct. Biol. 2014, 14, 10).

PTPN11 is widely expressed in most tissues and plays a regulatory role in various cell signaling events that are important for a diversity of cell functions that includes proliferation, differentiation, cell cycle maintenance, epithelial-mesenchymal transition (EMT), mitogenic activation, metabolic control, transcription regulation, and cell migration, through multiple signaling pathways including the Ras-MAPK, the JAK-STAT or the PI3K-AKT pathways (Tajan, M. et. al. Eur. J. Medical Genetics, 2015, 58, 509-525. Prahallad, A. et. al. Cell Reports, 2015, 12, 1978-1985).

Additionally there is growing evidence that PTPN11/SHP2 is implicated in immune evasion during tumorigenesis, and hence a SHP2 inhibitor could stimulate the immune response in cancer patients (Cancer Res. 2015 Feb. 1; 75(3):508-18. T Yokosuka T, J Exp Med. 2012, 209(6), 1201. S Amarnath Sci Transl Med. 2011, 3, 111ra120. T Okazaki, PNAS 2001, 98:24, 13866-71).

Substituted pyrimin-4(3H)-one compounds refer to a class of compounds having inhibitory activities against PTPN11/SHP2, as disclosed in International Patent Application No. PCT/US2019/045903 filed Aug. 9, 2019, and represented by the following formula:

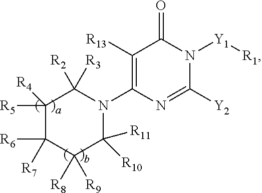

or a pharmaceutically acceptable salt, hydrate, solvate, stereoisomer, conformational isomer, tautomer, or a combination thereof, wherein the subscripts a and b, Y₁, Y₂, and R₁, R₂, R₃, R₄, R₅, R₆, R₇, R₈, R₉, R₁₀, R₁₁, and R₁₃ are as provided in PCT/US2019/045903, which is incorporated herein in its entirety for all purposes. In particular, the substituted pyrimin-4(3H)-one compound is represented by formula (I):

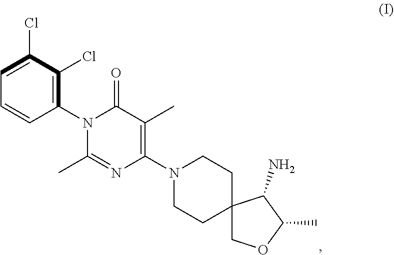

or a pharmaceutically acceptable salt, hydrate, solvate, stereoisomer, conformational isomer, tautomer, or a combination thereof. In certain embodiments, the compound of formula (I) is Compound (10b), represented by the formula:

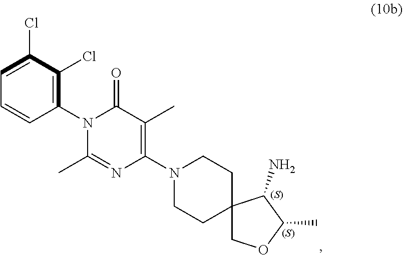

having the name of 6-((3S,4S)-4-amino-3-methyl-2-oxa-8-azaspiro[4.5]decan-8-yl)-3-(Rₐ)-(2,3-dichlorophenyl)-2,5-dimethylpyrimidin-4 (3H)-one.

The compound of formula (I), in particular formula (10b), is a potent, selective, orally active allosteric inhibitor of Src Homology-2 Phosphatase (SHP2) (also known as protein tyrosine phosphatase non-receptor type 11 (PTPN11)), a tyrosine phosphatase that plays a key role in the receptor tyrosine kinase (RTK)-mediated mitogen activated protein kinase (MAPK) signal transduction pathway (Matozaki, 2009). Key components of the MAPK pathway include the small GTPase RAS, the serine/threonine-protein kinase RAF, mitogen-activated protein kinase (MEK) and extracellular signal activated kinase (ERK). In cells, SHP2 binds to phosphorylated tyrosine residues in the intracellular domain of RTKs such as the Epidermal Growth Factor Receptor (EGFR), leading to activation of the downstream MAPK signaling pathway.

RTKs and the MAPK pathway function to relay external growth-promoting signals from the cell surface to the nucleus in many cell types. RTKs and components of the MAPK pathway, such as RAS and RAF, are frequently activated by mutation in human cancer, resulting in constitutive pathway activation. Several RTK and MAPK pathway inhibitors have been approved for the treatment of solid tumors in which activation of these pathways is the oncogenic driver, including inhibitors of RTKs such as EGFR (e.g., erlotinib, gefitinib, afatinib, dacomitinib, osimertinib and others), Anaplastic Lymphoma Kinase (ALK) (e.g., crizotinib, ceritinib and others) for EGFR-mutated and ALK-mutated non-small cell lung cancer (NSCLC), respectively, and MEK inhibitors (e.g., trametinib, cobimetinib and binimetinib) and BRAF inhibitors (e.g., vemurafenib, dabrafenib, encorafenib) for BRAF-mutated melanoma. In addition, various ERK inhibitors and KRAS inhibitors are in clinical trials, and a first KRAS G12C inhibitor, sotorasib, was approved in 2021. However, resistance to each of these pathway inhibitors has been observed in both clinical and nonclinical studies, frequently driven by compensatory activation or upregulation of other components of the pathway, including RTKs (Mainardi et al 2018; Ruess et al 2018).

Recent studies, including studies with the compound of formula (I) or (10b), have shown that SHP2 inhibition can potentiate the activity of RTK, MEK and KRAS inhibitors in nonclinical tumor models. These data suggest that inhibition of SHP2 has the potential to inhibit the growth of tumors that are dependent on activation of RTKs and/or harbor certain oncogenic RAS mutations (e.g., KRAS^{G12C}), including tumors in which adaptive resistance to therapeutic inhibitors of these pathways has developed.

SHP2 inhibition has the potential to become a backbone combination drug across a myriad of cancers. However, there remains a need for effective and safe therapeutic agents as, e.g., monotherapy agents in patients whose tumors display RTK and MAPK pathway alterations. The present disclosure provides such need.

### BRIEF SUMMARY

The present disclosure provides methods of treating cancer (e.g., solid tumors). In some embodiments, the methods include administering a SHP2 inhibitor represented by formula (I) (e.g., compound (10b), as described herein) to a subject in need thereof. In some embodiments, the subject has a cancer such as a solid tumor, such as non-small cell lung cancer (NSCLC). In some embodiments, the subject has one or more mutations, such as one or more mutations in the MAPK pathway and/or one or more mutations in PTPN11. In some embodiments, the subject has one or more mutations in the MAPK pathway other than a V600X mutation.

Accordingly, in one aspect, the present disclosure provides a method of treating cancer or a solid tumor. The method includes administering to a subject in need thereof a therapeutically effective amount of a compound represented by formula (I):

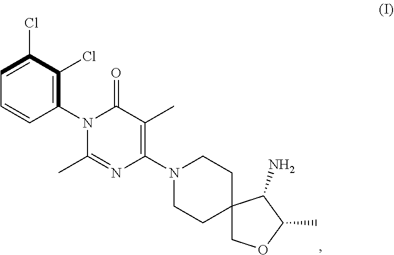

or a pharmaceutically acceptable salt, hydrate, solvate, stereoisomer, conformational isomer, tautomer, or a combination thereof, wherein the subject has (i) one or more mutations in MAPK pathway, provided that the one or more mutations in MAPK pathway are other than a BRAF mutation including V600X mutation, and/or (ii) one or more mutations in PTPN11.

In some embodiments, the solid tumor is an advanced or metastatic KRAS G12C-positive non-small cell lung cancer (NSCLC). In some embodiments, the solid tumor is an advanced or metastatic KRAS G12C-positive solid tumor, provided that the solid tumor is other than non-small cell lung cancer (NSCLC). In some embodiments, the solid tumor is an advanced or metastatic NF1 loss-of-function (LOF) solid tumor. In some embodiments, the solid tumor is an advanced or metastatic EGFR-positive non-small cell lung cancer (NSCLC), optionally progressed on a standard of care EGFR tyrosine kinase inhibitor (TKI) therapy such as osimertinib, erlotinib, afatinib, gefitinib, or dacomitinib. In some embodiments, the solid tumor is an advanced or metastatic KRAS mutant solid tumor. In some embodiments, the solid tumor is an advanced or metastatic BRAF class II/II mutant solid tumor. In some embodiments, the cancer or solid tumor is chordoma or notochord sarcoma.

### BRIEF DESCRIPTION OF THE DRAWINGS

FIGS. 1A-1B show representative dose-response curves of Compound (10b) in an in vitro enzymatic assay. FIG. 1A: enzymatic activity of wild-type SHP2; and FIG. 1B: enzymatic activity of SHP2 phosphatase domain.

FIGS. 2A-2B show PK/PD relationship and IC₅₀ determination in KYSE-520 treated with Compound (10b). FIG. 2A: PK/PD relationship; and FIG. 2B: IC₅₀ determination.

FIG. 3 shows mRNA levels of MPAS-plus signature genes in KYSE-520 xenograft tumors in mice at 4-hour, 16-hour, and 24-hour after either a single dose or five doses of Compound (10b) at 25 mg/kg or at 100 mg/kg QD PO.

FIG. 4 shows a dose relation of Compound (10b) exposure in male NOD/SCID mice after single oral dose and the duration over in vitro pERK IC₅₀ of the NSCLC HCC827 cell line.

FIG. 5 shows monotherapy anti-tumor activity of Compound (10b) in female BALB/c nude mice bearing the non-small cell lung cancer (NSCLC) HCC827 cell line-derived xenograft (CDX) model.

FIGS. 6A-6C show cell viability effects, pERK inhibition, and DUSP6 inhibition in NCI-H358 and KYSE-520 cell lines, following Compound (10b) treatment. FIG. 6A: 3D Viability; FIG. 6B: pERK inhibition; and FIG. 6C: DUSP6 inhibition.

FIG. 7 shows monotherapy anti-tumor activity of Compound (10b) in all CDX models.

FIG. 8 shows a design of a Phase 1/1B first-in-human study of the SHP2 inhibitor Compound (10b) in patients with advanced solid tumors. Expansion cohorts A-D includes advanced or metastatic KRAS G12C mutant NSCLC (Cohort A), advanced or metastatic KRAS G12C mutant non-NSCLC solid tumor (Cohort B), advanced or metastatic NF1 LOF solid tumor (Cohort C), and advanced or metastatic EGFR-mutant NSCLC that progressed on standard of care EGFR TKI therapies, with no available standard of care or curative therapies, including, e.g., advanced or metastatic osimertinib resistant NSCLC (Cohort D). Abbreviations: BOIN-Bayesian optimal interval design; EGFR-epidermal growth factor receptor; FE-food effect; IP-investigational product; LOF=loss-of-function; NSCLC=non-small cell lung cancer; PK=pharmacokinetic; RP2D=recommended phase 2 dose; TKI=tyrosine kinase inhibitor. Enrollment into Dose Escalation will occur first, followed by concurrent and independent enrollment into Dose Expansion and an optional FE/PK sub-study. The dose of IP administered in Dose Expansion and the optional FE/PK sub-study (i.e., RP2D), can be adjusted based on an SRC decision following review of the totality of the data. The FE/PK Study can be performed at selected centers only. Enrollment into the 2 PK cohorts will be at the sponsor's discretion.

FIG. 9 shows dose escalation of the Phase 1/1B first-in-human study. Abbreviations: EOT=end of treatment; IP=investigational product; LD=last dose; QD=once daily; SRC=Safety Review Committee. The treatment period will consist of sequential consecutive 28-day treatment cycles, with no drug holidays. In each treatment cycle, IP (Compound (10b)) will be taken QD. Absent a reason to interrupt dosing, a new cycle will be initiated immediately upon completion of the prior cycle. With each dose cohort, patients will be monitored for dose-limiting toxicities during the first cycle of treatment.

FIG. 10 shows dose expansion of the Phase 1/1B first-in-human study. Expansion cohorts A-D includes advanced or metastatic KRAS G12C mutant NSCLC (Cohort A), advanced or metastatic KRAS G12C mutant non-NSCLC solid tumor (Cohort B), advanced or metastatic NF1 LOF solid tumor (Cohort C), and advanced or metastatic EGFR-mutant NSCLC that progressed on standard of care EGFR TKI therapies, with no available standard of care or curative therapies, including, e.g., advanced or metastatic osimertinib resistant NSCLC (Cohort D). Abbreviations: EGFR=epidermal growth factor receptor; LOF=loss-of-function; NSCLC=non-small cell lung cancer; RP2D=recommended phase 2 dose; SRC=Safety Review Committee; TK=tyrosine kinase. The treatment period will consist of sequential consecutive 28-day treatment cycles, with no drug holidays. In each treatment cycle, IP (Compound (10b)) will be taken QD. Absent a reason to interrupt dosing, a new cycle will be initiated immediately upon completion of the prior cycle. The dose of IP administered in Dose Expansion (i.e., RP2D), can be adjusted based on an SRC decision following review of the totality of the data.

FIG. 11 shows a sample FE/PK sub-study of a Phase 1/1B first-in-human study. Abbreviations: EOT=end of treatment; FE=food effect; IP=investigational product; LD=last dose; PK=pharmacokinetic; QD-once daily; RP2D=recommended phase 2 dose; SRC=Safety Review Committee. The treatment period will consist of sequential consecutive 28-day treatment cycles, with no drug holidays. In each treatment cycle, IP (Compound (10b)) will be taken QD. Absent a reason to interrupt dosing, a new cycle will be initiated immediately upon completion of the prior cycle. The dose of IP administered in the sub-study (i.e., RP2D), can be adjusted based on an SRC decision following review of the totality of the data. The FE/PK Study can be performed at selected centers only. Enrollment into the 2 PK cohorts will be at the sponsor's discretion. The 3-day washout for the FE Cohort has a −1/+3 day window.

FIGS. 12A-12B show mean plasma concentrations of compound (10b) in Cohorts 1-5 of Example 6. FIG. 12A: Day 1 of Cycle 1 (C1D1); and FIG. 12B: Day 1 of Cycle 2 (C2D1). Dotted line represents in vivo IC₅₀ (1.5 μM) and predicted efficacious Cmax (5.3 μM).

FIGS. 13A-13E show pERK inhibition of compound (10b) in Cohorts 1-5 of Example 6. FIG. 13A: Cohort 1; FIG. 13B: Cohort 2: FIG. 13C: Cohort 3; FIG. 13D: Cohort 4; and FIG. 13E: Cohort 5. Dotted line represents IC₅₀ of 1.5 μM. Bars represent PD (% pERK) and filled circles represent PK.

FIG. 14 shows a design for the Phase 1/1B first-in-human study described in Examples 7 and 8. The study includes a Dose Escalation portion including 6 cohorts. Patients enrolled in the Dose Escalation portion has MAPK pathway alterations such as KRAS G12C mutations or EGFR mutations, provided that the subjects do not have activating mutations such as BRAF V600X or RAS Q61X. The study also includes a Dose Expansion portion. Both cohorts of the Dose Expansion portion includes patients with advanced or metastatic KRAS mutant solid tumors, advanced or metastatic NF1 LOF solid tumors, or advanced or metastatic BRAF class II/III mutant solid tumors. Cohort 1 of the Dose Expansion portion will identify the RP2D, while Cohort 2 of the Dose Expansion portion will use a single dose (amount to be determined). Abbreviations: LOF=loss-of-function; RP2D=recommended phase 2 dose; and SRC=Safety Review Committee. The treatment period will consist of sequential consecutive 28-day treatment cycles, with no drug holidays. In each treatment cycle, Compound (10b) will be taken QD orally. Absent a reason to interrupt dosing, a new cycle will be initiated immediately upon completion of the prior cycle. The dose of Compound (10b) administered in Dose Expansion (i.e., RP2D), can be adjusted based on an SRC decision following review of the totality of the data.

### DETAILED DESCRIPTION

### I. General

The present disclosure provides methods of treating cancer or a solid tumor (e.g., an advanced or metastatic solid tumor) with a therapeutically effective amount of a compound of formula (I), in particular Compound (10b) in a subject. The subject can have one or more mutations in the MAPK pathway. The one or more mutations in the MAPK pathway may exclude activating mutations such as BRAF V600X mutations, mutations in PTPN11, and/or RAS Q61X mutations. The solid tumor can be an advanced or metastatic KRAS mutant solid tumor (e.g., KRAS G12C-positive non-small cell lung cancer (NSCLC) or an advanced or metastatic KRAS G12C-positive solid tumor other than NSCLC); an advanced or metastatic NF1 loss-of-function (LOF) solid tumor or BRAF class II/III mutant solid tumor; or an advanced or metastatic EGFR-positive NSCLC, such as an EGFR-mutant NSCLC that progressed on a standard of care EGFR tyrosine kinase inhibitor (TKI) therapy or may have no available standard of care or curative therapy, such as advanced or metastatic osimertinib resistant NSCLC. The cancer or tumor can also be sarcomas, such as chordoma.

### II. Definitions

As used herein, the terms below have the meanings indicated.

“Comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 1 mg to 3 mg (milligram),” which is intended to include 1 mg, 3 mg, and everything in between to any number of significant figures (e.g., 1.255 mg, 2.1 mg, 2.9999 mg, etc.).

“Salt” refers to acid or base salts of the compounds of the present disclosure. Illustrative examples of pharmaceutically acceptable acid addition salts are mineral acid (hydrochloric acid, hydrobromic acid, phosphoric acid, and the like) salts and organic acid (acetic acid, propionic acid, glutamic acid, citric acid and the like) salts. Examples of pharmaceutically acceptable base addition salts include sodium, potassium, calcium, ammonium, organic amino, or magnesium salt, or a similar salt. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

“Solvate” refers to a compound provided herein or a salt thereof, that further includes a stoichiometric or non-stoichiometric amount of solvent bound by non-covalent intermolecular forces.

“Hydrate” refers to a compound that is complexed to a water molecule. The compounds of the present disclosure can be complexed with ½ water molecule or from 1 to 10 water molecules.

Asymmetric centers exist in the compounds disclosed herein. These centers are designated by the symbols “R” or “S,” depending on the configuration of substituents around the chiral carbon atom. It should be understood that the present disclosure encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method. Starting compounds of particular stereochemistry are either commercially available or can be made and resolved by various techniques. Additionally, compounds may exist as tautomers; all tautomeric isomers are provided by this disclosure. Additionally, the compounds disclosed herein can exist in unsolvated as well as solvated forms with pharmaceutically acceptable solvents such as water, ethanol, and the like. In general, the solvated forms are considered equivalent to the unsolvated forms.

“Tautomer”, as use herein, alone or in combination, refers to one of two or more isomers that rapidly interconvert. Generally, this interconversion is sufficiently fast so that an individual tautomer is not isolated in the absence of another tautomer. The ratio of the amount of tautomers can be dependent on solvent composition, ionic strength, and pH, as well as other solution parameters. The ratio of the amount of tautomers can be different in a particular solution and in the microenvironment of a biomolecular binding site in said solution. Examples of tautomers include keto/enol, enamine/imine, and lactam/lactim tautomers. Additional examples of tautomers also include 2-hydroxypyridine/2(1H)-pyridone and 2-aminopyridine/2(1H)-iminopyridone tautomers.

Conformational isomers exist in the compounds disclosed herein. When R₁ is aryl or heteroaryl in the formula:

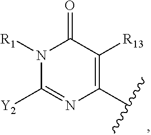

the aryl or heteroaryl group can orient in different conformations in relation to the pyrimidinone moiety, as represented by:

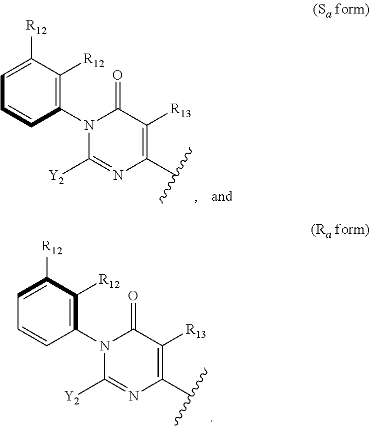

These forms are designated by the symbols “Sₐ” or “Rₐ”, depending on the conformation of the aryl or heteroaryl group in relation to the pyrimidinone moiety. Examples of “Sₐ” and “Rₐ” forms can be found in Examples 1-20 of International Patent Application No. PCT/US2019/045903, which is incorporated herein in its entirety for all purposes. The compound of formula (10b) is substantially in a “Rₐ” form.

“Pharmaceutically acceptable” refers to those compounds (salts, hydrates, solvates, stereoisomers, conformational isomers, tautomers, etc.) which are suitable for use in contact with the tissues of patients without undue toxicity, irritation, and allergic response, are commensurate with a reasonable benefit/risk ratio, and are effective for their intended use. The compounds disclosed herein can exist as pharmaceutically acceptable salts, as defined and described herein.

“PTPN11 inhibitor” is used herein to refer to a compound that exhibits an IC₅₀ with respect to PTPN11 activity of no more than about 100 micromolar (μM) and more typically not more than about 50 μM, as measured in the PTPN11 assay described generally in International Patent Application No. PCT/US2019/045903 (e.g., the enzymatic activity of recombinant human PTPN11 proteins of Example 21) or the enzymatic activity of recombinant human SHP2 protein of Example 1. “IC₅₀” is that concentration of inhibitor which reduces the activity of an enzyme (e.g., PTPN11) to half-maximal level. In certain embodiments, compounds disclosed in PCT/US2019/045903 exhibit an IC₅₀ of no more than about 10 μM for inhibition of PTPN11; in further embodiments, compounds exhibit an IC₅₀ of no more than about 1 μM for inhibition of PTPN11; in yet further embodiments, compounds exhibit an IC₅₀ of not more than about 200 nM for inhibition of PTPN11; in yet further embodiments, compounds exhibit an IC₅₀ of not more than about 100 nM for inhibition of PTPN11; and in yet further embodiments, compounds exhibit an IC₅₀ of not more than about 50 nM for inhibition of PTPN11, as measured in the PTPN11 assay described therein. In certain embodiments, the compound of formula (I) or (10b) exhibits an IC₅₀ of no more than 50 nM for inhibition of PTPN11 (e.g., a PTPN11-E76K mutant enzyme).

“Composition,” as used herein, is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present disclosure include, but are not limited to, binders, fillers, glidants, disintegrants, surfactants, lubricants, coatings, sweeteners, flavors, and colors. One of skill in the art will recognize that other pharmaceutical excipients are useful in the present disclosure.

“Tablet” refers to solid pharmaceutical formulations with and without a coating. The term “tablet” also refers to tablets having one, two, three or even more layers, wherein each of the before mentioned types of tablets may be without or with one or more coatings. In some embodiments, tablets of the present disclosure can be prepared by roller compaction or other suitable means known in the art. The term “tablet” also comprises mini, melt, chewable, effervescent, and orally disintegrating tablets. Tablets include the compound of formula (I) or (10b) and one or more pharmaceutical excipients (e.g., fillers, binders, glidants, disintegrants, surfactants, binders, lubricants, and the like). Optionally, a coating agent can be also included. For the purposes of calculating percent weight of the tablet formulation, the amount of coating agent is not included in the calculation. That is, the percent weights reported herein are of the uncoated tablet.

“Administering” refers to therapeutic provision of the compound or a form thereof to a subject, such as by oral administration.

“Patient” or “subject” refers to a living organism suffering from or prone to a disease or condition that can be treated by administration of a pharmaceutical composition as provided herein. Non-limiting examples include humans, non-human primates (e.g., monkeys), goats, pigs, sheep, cows, deer, horses, bovines, rats, mice, rabbits, hamsters, guinea pigs, cats, dogs, and other non-mammalian animals. In some embodiments, the subject is human. In some embodiments, a subject is an adult (e.g., at least 18 years of age).

“Therapeutically effective amount” refers to an amount of a compound or of a pharmaceutical composition useful for treating or ameliorating an identified disease or condition, or for exhibiting a detectable therapeutic or inhibitory effect. The exact amounts will depend on the purpose of the treatment, and will be ascertainable by clinicians, pharmacists, and the like (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Treat”, “treating,” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, assay (e.g., analysis of a fluid of a subject, such as blood, plasma, or urine), imaging analysis, neuropsychiatric exams, and/or a psychiatric evaluation.

“About” means a range of values including the specified value, which a person of ordinary skill in the art would consider reasonably similar to the specified value. In some embodiments, the term “about” means within a standard deviation using measurements generally acceptable in the art. In some embodiments, about means a range extending to +/−10% of the specified value. In some embodiments, about means the specified value.

Unless specifically indicated otherwise, the content of the compound of formula (I) or (10b) in, e.g., a tablet formulation is calculated based on the normalized weight of the compound of formula (I) or (10b) on a salt-free and anhydrous basis. That is, the salt and/or water content in the compound of formula (I) or (10b) is not included in the calculation.

“KRAS G12C inhibitor” as used herein refers to a compound which targets, decreases, or inhibits the synthesis or biological activity of KRAS (Kirsten rat sarcoma 2 viral oncogene homolog) by selectively modifying mutant cysteine 12 in G12C mutated KRAS. The KRAS G12C inhibitor may at least partially inhibit KRAS G12C kinase. The KRAS G12C inhibitor may be a selective KRAS G12C inhibitor (e.g., having greater selectivity for KRAS having a G12C mutation over KRAS having another mutation such as a G12D mutation). In those cases, the selective KRAS G12C inhibitor may have high potency for KRAS G12C, along with low affinity for other KRAS mutations. The KRAS G12C inhibitor may be a covalent inhibitor (e.g., capable of covalently modifying cysteine 12). The KRAS G12C inhibitor may be a noncovalent inhibitor. The KRAS G12C inhibitor may bind to an inactive (“GDP”) form of KRAS. The KRAS G12C inhibitor may bind to an active (“GTP”) form of KRAS. The KRAS G12C inhibitor may bind to both inactive (“GDP”) and active (“GTP”) forms of KRAS. Examples of KRAS G12C inhibitors include sotorasib (AMG 510), adagrasib (MRTX-849), MRTX1257, ARS-853, ARS-1620, JNJ-74699157 (ARS-3248), JDQ443, GDC-6036, JAB-21822, BI 1823911, MK-1084, LY3537982, and LY3499446.

“KRAS-positive cancer” refers to a cancer with the KRAS gene rearranged, mutated, or amplified. “KRAS G12C-positive cancer” refers to a cancer with the KRAS G12C gene rearranged, mutated, or amplified.

“A cancer resistant to a KRAS inhibitor” and/or “a cancer that is a KRAS-positive cancer resistant to a KRAS inhibitor” refer to a cancer or tumor that either fails to respond favorably to treatment with a prior KRAS inhibitor, or alternatively, recurs or relapses after responding favorably to a KRAS inhibitor. “A cancer resistant to a KRAS G12C inhibitor” and/or “a cancer that is a KRAS G12C-positive cancer resistant to a KRAS G12C inhibitor” refer to a cancer or tumor that either fails to respond favorably to treatment with a prior KRAS G12C inhibitor, or alternatively, recurs or relapses after responding favorably to a KRAS G12C inhibitor.

“Neurofibromatosis type 1 tumor manifestation e.g., neurofibromas that are resistant to a MEK inhibitor” or “an NF1 tumor resistant to a MEK inhibitor” refers an NF1 that either fails to respond favorably to treatment with a prior MEK inhibitor, or alternatively, recurs or relapses after responding favorably to a MEK inhibitor.

“EGFR inhibitor” as used herein refers to a compound which targets, decreases, or inhibits the synthesis or biological activity of epidermal growth factor receptor (EGFR). The EGFR inhibitor may at least partially inhibit EGFR kinase. The EGFR inhibitor may be a selective EGFR inhibitor. In those cases, the selective EGFR inhibitor may have high potency for EGFR, along with low affinity for other related kinases. Examples of EGFR inhibitors include erlotinib, cetuximab, panitumumab, vandetanib, afatinib, gefitinib, osimertinib, necitumumab, brigatinib, neratinib, dacomitinib, amivantamab (JNJ-61186372), mobocertinib (TAK-788), BLU-945, varlitinib, tarloxitinib, poziotinib, and lapatinib.

“EGFR-positive cancer” refers to a cancer with the EGFR gene rearranged, mutated, or amplified.

“A cancer resistant to an EGFR inhibitor” and “a cancer that is an EGFR-positive cancer resistant to an EGFR inhibitor” refer to a cancer or tumor that either fails to respond favorably to treatment with a prior EGFR inhibitor, or alternatively, recurs or relapses after responding favorably to an EGFR inhibitor.

“A,” “an,” or “a (n)”, when used in reference to a group of substituents or “substituent group” herein, mean at least one. For example, where a compound is substituted with “an” alkyl or aryl, the compound is substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

### III. Methods

In one aspect, the present disclosure provides a method of treating cancer or a solid tumor. The method includes administering to a subject in need thereof a therapeutically effective amount of a compound represented by formula (I):

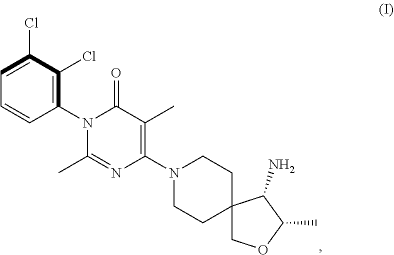

or a pharmaceutically acceptable salt, hydrate, solvate, stereoisomer, conformational isomer, tautomer, or a combination thereof. In some embodiments, the subject has one or more mutations in the MAPK pathway, such as a mutation other than a BRAF mutation comprising V600X mutation. In some embodiments, the subject has one or more mutations in PTPN11. In some embodiments, the compound of formula (I) is administered as a monotherapy agent.

### III-1: Compound of Formula (I)

The compound of formula (I) can be in a pharmaceutically acceptable salt form or in a neutral form, each of which is optionally in a solvate or a hydrate form.

In some embodiments, the compound of formula (I) is in a pharmaceutically acceptable salt form. In some embodiments, a pharmaceutically acceptable acid addition salt of the compound of formula (I) is represented by formula (Ia):

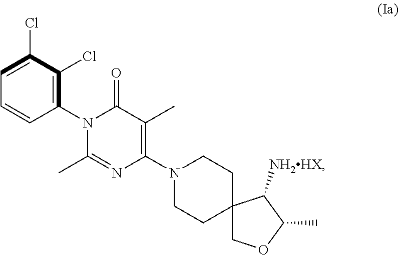

wherein HX is a pharmaceutically acceptable acid addition.

Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like.

In some embodiments, the compound of formula (I) is in a neutral form.

In some embodiments, the compound of formula (I) has a substantially moiety of 6-((3S,4S)-4-amino-3-methyl-2-oxa-8-azaspiro[4.5]decan-8-yl) with stereochemistry as shown in formula (10b):

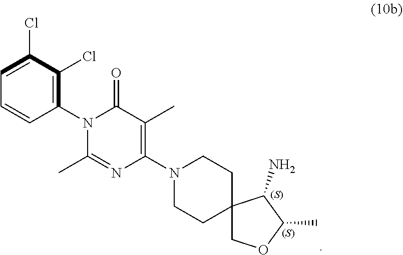

In some embodiments, the compound of formula (I) is substantially in a Ra conformation as shown in formula (10b):

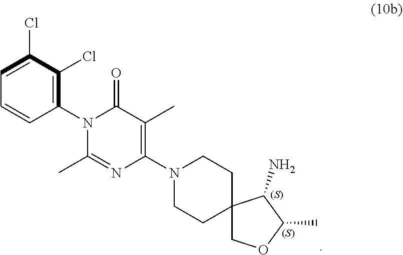

In some embodiments, the compound of formula (I) is represented by formula (10b):

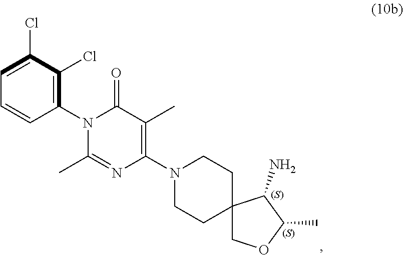

having the name of 6-((3S,4S)-4-amino-3-methyl-2-oxa-8-azaspiro[4.5]decan-8-yl)-3-(Ra)-(2,3-dichlorophenyl)-2,5-dimethylpyrimidin-4(3H)-one.

In some embodiments, the compound of formula (I) or (10b) is in a neutral form.

In some embodiments, the compound of formula (I) includes one or more corresponding enantiomer, diastereomers, and/or conformational isomers of the compound of formula (10b), as represented by formulae, respectively:

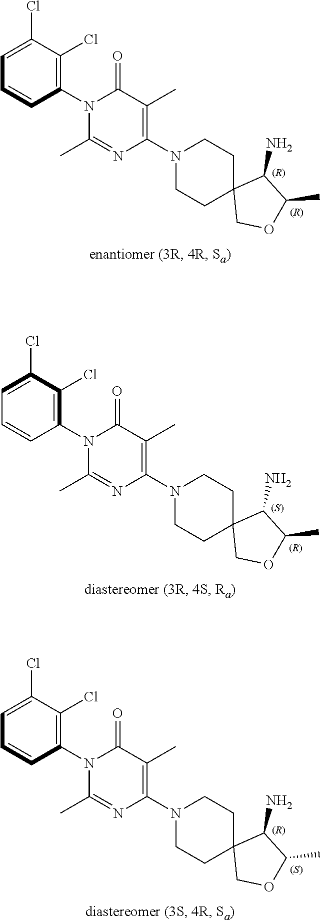

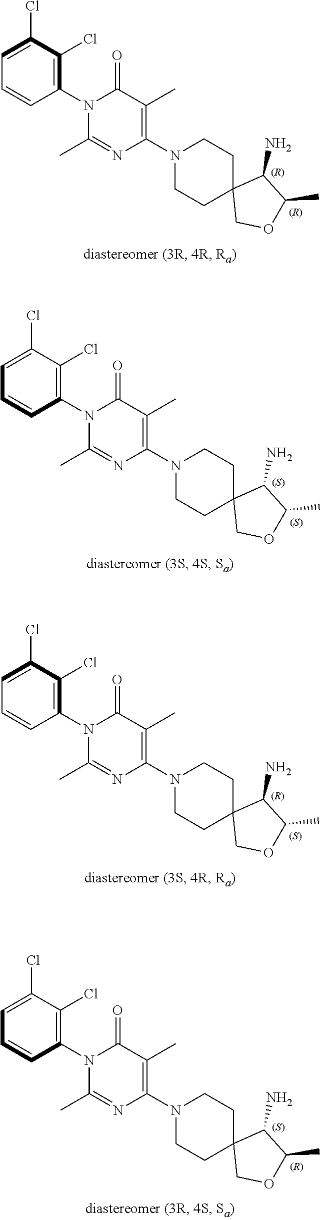

In some embodiments, the compound of formula (10b) has a purity of at least about 95 area % determined by a chiral high-performance liquid chromatography (HPLC). In some embodiments, the compound of formula (10b) has a purity of from about 95 area % to about 99 area %, from about 96 area % to about 99 area %, from about 97 area % to about 99 area %, or from about 98 area % to about 99 area %, determined by a chiral high-performance liquid chromatography (HPLC). In some embodiments, the compound of formula (10b) has a purity of from about 98 area % to about 99 area %.

In some embodiments, the compound of formula (I) includes one or more corresponding enantiomer, diastereomers, and/or conformational isomers of the compound of formula (10b), as represented by the formulae above; and a total of the one or more isomers is no more than about 5 area % determined by a chiral high-performance liquid chromatography (HPLC).

In some embodiments, the corresponding enantiomer, diastereomers, and/or conformational isomers of the compound of formula (10b) are present in the compound of formula (I) meet acceptance criteria as follows: enantiomer (3R, 4R, Sₐ)≤0.5 area %; diastereomer (3R, 4S, Rₐ)≤1.2 area %; diastereomer (3S, 4R, Sₐ)≤0.5 area %; diastereomer (3R, 4R, Rₐ)≤0.5 area %; diastereomer (3S, 4S, Sₐ)≤0.5 area %; diastereomer (3S, 4R, Rₐ)≤0.5 area %; and diastereomer (3R, 4S, Sₐ)≤0.5 area %, each of which is determined by a chiral high-performance liquid chromatography (HPLC). In some embodiments, the compound of formula (10b) has a purity of at least about 95 area %, wherein enantiomer (3R, 4R, Sₐ)<0.5 area %; diastereomer (3R, 4S, Rₐ)<1.2 area %; diastereomer (3S, 4R, Sₐ)<0.5 area %; diastereomer (3R, 4R, Rₐ)<0.5 area %; diastereomer (3S, 4S, Sₐ)<0.5 area %; diastereomer (3S, 4R, Rₐ)<0.5 area %; and diastereomer (3R, 4S, Sₐ)<0.5 area %, each of which is determined by a chiral high-performance liquid chromatography (HPLC). In some embodiments, the compound of formula (10b) has a purity of from about 95 area % to about 99 area %, from about 96 area % to about 99 area %, from about 97 area % to about 99 area %, or from about 98 area % to about 99 area %, wherein enantiomer (3R, 4R, Sₐ)<0.5 area %; diastereomer (3R, 4S, Rₐ)<1.2 area %; diastereomer (3S, 4R, Sₐ)<0.5 area %; diastereomer (3R, 4R, Rₐ)<0.5 area %; diastereomer (3S, 4S, Sₐ)<0.5 area %; diastereomer (3S, 4R, Rₐ)<0.5 area %; and diastereomer (3R, 4S, Sₐ)<0.5 area %, each of which is determined by a chiral high-performance liquid chromatography (HPLC). In some embodiments, the compound of formula (10b) has a purity of from about 98 area % to about 99 area %, wherein enantiomer (3R, 4R, Sₐ) is not detected; diastereomer (3R, 4S, Rₐ) is about 0.86 area %; diastereomer (3S, 4R, Sₐ) is not detected; diastereomer (3R, 4R, Rₐ) is about 0.07 area %; diastereomer (3S, 4S, Sₐ) is not detected; diastereomer (3S, 4R, Rₐ) is not detected; and diastereomer (3R, 4S, Sₐ) is not detected, each of which is determined by a chiral high-performance liquid chromatography (HPLC).

In some embodiments, the compound of any one of formulae (I), (Ia), and (10b) is in a solvate and/or a hydrate form.

### III-2: Subject

In some embodiments, the subject is human. In some embodiments, the subject is under the care of a medical practitioner, such as a physician. In some embodiments, the subject has been diagnosed with a cancer (e.g., as described herein). In some embodiments, the subject has a cancer including a solid tumor.

The subject can have an advanced (e.g., primary or recurrent) or metastatic cancer or solid tumor with MAPK-pathway alterations (e.g., MAPK-pathway alterations excluding BRAF V600X mutations) as assessed by molecular diagnostic using an appropriate clinically validated and/or FDA approved test and with no available standard of care or curative therapies.

In some embodiments, the subject has a mutation in a RAS protein (e.g., KRAS, NRAS, or HRAS). In some embodiments, the subject has a mutation in a RAS protein other than a Q61X mutation. In some embodiments, the subject has a mutation in KRAS other than a KRAS Q61X mutation (e.g., the subject has a cancer characterized by a mutation in KRAS other than KRAS Q61X). In some embodiments, a KRAS protein comprises a G12C, G12D, G12S, G12V, G12R, G12A, G13D, G13A, G13C, G13R, G13S, and/or G13V mutation (e.g., the subject has a cancer characterized by a G12C, G12D, G12S, G12V, G12R, G12A, G13D, G13A, G13C, G13R, G13S, and/or G13V mutation in KRAS). In some embodiments, the subject has a KRAS mutation including a KRAS G12C mutation. In some embodiments, the subject has a KRAS mutation including a KRAS G12A mutation, a KRAS G12D mutation, a KRAS G12F mutation, a KRAS G12I mutation, a KRAS G12L mutation, a KRAS G12R mutation, a KRAS G12S mutation, a KRAS G12V mutation, a KRAS G12Y mutation, a KRAS G13D mutation, or a combination thereof (e.g., the subject has a cancer characterized by a KRAS mutation including a KRAS G12C mutation, a KRAS G12A mutation, a KRAS G12D mutation, a KRAS G12F mutation, a KRAS G12I mutation, a KRAS G12L mutation, a KRAS G12R mutation, a KRAS G12S mutation, a KRAS G12V mutation, a KRAS G12Y mutation, a KRAS G13D mutation, or a combination thereof).

In some embodiments, the subject has one or more mutations in the MAPK pathway, provided that the one or more mutations in the MAPK pathway are other than a BRAF mutation comprising V600X mutation. In some embodiments, the subject has one or more mutations in the MAPK pathway selected from the group consisting of one or more mutations in NRAS, HRAS, CRAF, BRAF, NRAF, MAPK/ERK, MAPK/MEK, NF1, IGFR, PDGFR, VEGFR, FGFR, CCKR, NGFR, EphR, AXLR, KEAP-1, TIE receptor, RYK receptor, DDR receptor, RET receptor, ROS receptor, LTK receptor, ROR receptor, and MuSK receptor. In some embodiments, the subject has a mutation in NRAS. In some embodiments, the subject has a mutation in HRAS. In some embodiments, the subject has a mutation in CRAF. In some embodiments, the subject has a mutation in BRAF (except for V600X mutation). In some embodiments, the subject has a mutation in NRAF. In some embodiments, the subject has a mutation in MAPK/ERK. In some embodiments, the subject has a mutation in MAPKK/MEK. In some embodiments, the subject has a mutation in NF1. In some embodiments, the subject has a mutation in IGFR. In some embodiments, the subject has a mutation in PDGFR. In some embodiments, the subject has a mutation in VEGFR. In some embodiments, the subject has a mutation in FGFR. In some embodiments, the subject has a mutation in CCKR. In some embodiments, the subject has a mutation in NGFR. In some embodiments, the subject has a mutation in EphR. In some embodiments, the subject has a mutation in AXLR. In some embodiments, the subject has a mutation in KEAP-1 receptor. In some embodiments, the subject has a mutation in TIE receptor. In some embodiments, the subject has a mutation in RYK receptor. In some embodiments, the subject has a mutation in DDR receptor. In some embodiments, the subject has a mutation in RET receptor. In some embodiments, the subject has a mutation in ROS receptor. In some embodiments, the subject has a mutation in LTK receptor. In some embodiments, the subject has a mutation in ROR receptor. In some embodiments, the subject has a mutation in MuSK receptor.

In some embodiments, the subject has a mutation in BRAF (except for V600X mutation). In some embodiments, the subject has a class II mutation in BRAF. In some embodiments, the subject has a class III mutation in BRAF.

In some embodiments, the subject has a mutation in EGFR. In some embodiments, subject has an EGFR mutation including an EGFR exon 19 deletion, exon 20 insertion, L858X mutation, T790X mutation, C797X mutation, G719X mutation, L861X mutation, S768X mutation, E709X mutation, or any combination thereof. In some embodiments, subject has an EGFR mutation including an EGFR exon 19 deletion, and/or exon 20 insertion. In some embodiments, subject has an EGFR exon 19 deletion. In some embodiments, subject has an EGFR exon 20 insertion.

In some embodiments, the subject has a mutation in PTPN11. In some embodiments, the subject has a mutation in PTPN11 including E76K mutation. In some embodiments, the subject does not have a mutation in PTPN11. In some embodiments, the subject does not have an E76K mutation in PTPN11.

In some embodiments, the subject has a measurable disease according to response evaluation criteria in solid tumors (RECIST). In some embodiments, treatment of the subject with the compound of formula (I) or (10b) causes a measurable change in disease state according to RECIST.

In some embodiments, the subject has adequate organ functions including adequate hematological, renal, hepatic, and coagulating functions as defined and described below:

### Hematological

absolute neutrophil count (ANC)≥1,500/mcL;
platelets ≥100,000/mcL; and
hemoglobin ≥9 g/dL without transfusion for >2 weeks or erythropoiesis-stimulating agents (e.g., Epo, Procrit) for >6 weeks.

### Renal

Estimated glomerular filtration rate ≥60 mL/min/1.73 m² (calculated by the Chronic Kidney Disease Epidemiology Collaboration equation)

### Hepatic

Serum total bilirubin <2.0× institutional upper limit of normal (ULN) or <3.0× institutional ULN if the individual has a diagnosis of Gilbert syndrome or hemolytic anemia as confirmed by the investigator; and
Aspartate aminotransferase/serum glutamic-oxaloacetic transaminase (AST/SGOT) and/or alanine aminotransferase/serum glutamic-pyruvic transaminase (ALT/SGPT)≤2.5×ULN or ≤5×ULN in the presence of liver metastases.

### Coagulation

International normalized ratio (INR) or prothrombin time (PT)≤1.5×ULN unless the patient is receiving anticoagulant therapy and as long as PT or activated partial thromboplastin time (aPTT) is within the therapeutic range of intended use of anticoagulants; and
Activated partial thromboplastin time (aPTT)≤1.5×ULN unless the patient is receiving anticoagulant therapy and as long as PT or aPTT is within the therapeutic range of intended use of anticoagulants.

In some embodiments, prior to administration of a compound of formula (I) or (10b), the subject has not undergone treatment with any chemotherapy or other investigational therapy such as hormonal (including corticosteroids), biological, or targeted agents for ≥3 weeks; or the subject is at least 5 half-lives from hormonal (including corticosteroids), biological, or targeted agents, whichever is longer at the time of treatment initiation.

In some embodiments, prior to administration of a compound of formula (I) or (10b), the subject is not previously treated with a cancer therapy comprising a chemotherapy, a hormone therapy, an immunotherapy or biological therapy, a targeted therapy, or a combination thereof.

In some embodiments, the subject that has been or is on a cancer therapy including a chemotherapy, a hormone therapy, an immunotherapy or biological therapy, a targeted therapy, or a combination thereof is treated with a compound of formula (I) or (10b) if the subject discontinues such cancer therapy (e.g., a chemotherapy, a hormone therapy, an immunotherapy or biological therapy, a targeted therapy, or a combination thereof) for a period of at least about three weeks (such as at least about four weeks) or five (5) half-lives of an agent used in the cancer therapy, whichever is longer prior to initiation of the treatment with the compound of formula (I) or (10b).

In some embodiments, the subject does not have one or more additional activating mutations in PTPN11 (SHP2), MEK, or RAS (e.g., NRAS, HRAS, KRAS; such as a Q61X mutation).

In some embodiments, the subject does not have an additional malignancy that is progressing or requires an active treatment, wherein the additional malignancy includes basal cell carcinoma of the skin, squamous cell carcinoma of the skin that has undergone potentially curative therapy or in situ cervical cancer. In some embodiments, the subject has not had an additional malignancy within the previous 3 years that has progressed or requires an active treatment, wherein the additional malignancy is other than non-melanomatous skin cancer, superficial urothelial carcinoma, in situ cervical cancer, or any other curatively treated malignancy that is not expected to require treatment for recurrence during the course of the study.

In some embodiments, the subject does not have a primary central nervous system (CNS) tumor, an active CNS metastasis, and/or carcinomatous meningitis. In some embodiments, the subject does not have a primary central nervous system (CNS) tumor. In some embodiments, the subject does not have an active CNS metastasis, and/or a carcinomatous meningitis.

In some embodiments, the subject, who has brain metastases, is treated with a compound of formula (I) or (10b), if i) the brain metastases are stable (without evidence of progression by imaging for at least four weeks prior to administration of a compound of formula (I) or (10b) and any neurologic symptoms have returned to baseline); ii) the subject has no evidence of new or enlarging brain metastases; and iii) the subject is not using steroids and/or anti-seizures medications for at least 7 days prior to prior to administration of a compound of formula (I) or (10b), provided that the subject does not have carcinomatous meningitis.

In some embodiments, the subject is not previously treated with a SHP2 inhibitor (e.g., TNO-155, RMC-4630, RLY-1971, JAB-3068, JAB-3312, PF-07284892, or ERAS601). In some embodiments, the subject is not previously treated with the compound of formula (I) or (10b). In some embodiments, the subject is previously treated with a SHP2 inhibitor. In some embodiments, the subject is previously treated with the compound of formula (I) or (10b).

In some embodiments, prior to administration of a compound of formula (I) or (10b), the subject has not previously taken or is not taking one or more of strong or moderate inducers or inhibitors of CYP3A4 and/or P-gp inducers or inhibitors (including herbal supplements) (e.g., Appendix 3). In some embodiments, prior to administration of a compound of formula (I) or (10b), the subject has not previously taken or is not taking one or more of strong or moderate inducers or inhibitors of cytochrome P450 (CYP) 3A4 or P-glycoprotein (P-gp) inducers (including herbal supplements or food products containing grapefruit juice, star fruit, or Seville oranges) within 14 days or 5 half-lives (whichever is longer) of the concomitant medication.

In some embodiments, the subject has taken or is taking one or more of strong or moderate inducers or inhibitors of CYP3A4 and/or P-gp inducers or inhibitors (including herbal supplements) (e.g., Appendix 3) is treated with a compound of formula (I) or (10b) if the subject discontinues such treatment for a period of at least about five (5) half-lives prior to initiation of the treatment with the compound of formula (I) or (10b) and during the treatment period of the compound of formula (I) or (10b).

In some embodiments, prior to administration of a compound of formula (I) or (10b), the subject has not previously taken or is not taking a drug that is a known substrate of P-gp, BCRP, OATP1B1, OATP1B3, MATE1, and/or MATE2-K transporters.

In some embodiments, the subject has taken or is taking a drug that is a known substrate of P-gp, BCRP, OATP1B1, OATP1B3, MATE1, and/or MATE2-K transporters is treated with a compound of formula (I) or (10b) if the subject discontinues such treatment prior to initiation of the treatment with the compound of formula (I) or (10b) and during the treatment period of the compound of formula (I) or (10b).

Further inclusion and exclusion criteria for subjects who may benefit from treatment with a compound of formula (I) or (10b), such as subjects enrolled in a Phase 1/1B first-in-human Study, are described in Example 7.

In some embodiments, the subject meets all of inclusion criteria of 1) to 10) as described in Example 7. In some embodiments, the subject meets all of inclusion criteria of 1) to 10) as described in Example 7, provided that the subject does not meet any one of exclusion criteria of 1) to 22) as described in Example 7.

### III-3: Cancer/Solid Tumor

In some embodiments, the cancer is selected from pancreatic cancer; colon cancer; rectal cancer; colorectal cancer; breast cancer; ovarian cancer; endometrial cancer; lung cancer; prostate cancer; cancers of the oral cavity and pharynx (lip, tongue, mouth, larynx, pharynx), esophagus, stomach, small intestine, large intestine, liver and biliary passages, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; cancers of the thyroid and other endocrine glands; Hodgkin's disease; non-Hodgkin's lymphomas; multiple myeloma; and hematopoietic malignancies including leukemias (Chronic Lymphocytic Leukemia (CLL), Acute Lymphocytic Leukemia (ALL), Chronic Myelogenous Leukemia (CML), Acute Myelogenous Leukemia (AML), and lymphomas including lymphocytic, granulocytic and monocytic lymphomas. In some embodiments, the cancer is selected from adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma, Kaposi's sarcoma, large cell carcinoma, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, paraganglioma, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, non-small cell lung carcinoma, squamous cell carcinoma, sweat gland carcinoma, synovioma, thyroid cancer, uveal melanoma, and Wilm's tumor. In some embodiments, the cancer is selected from breast cancer, lung cancer (e.g., non-small cell lung cancer), endometrial cancer, esophageal cancer, ovarian cancer, colorectal cancer, gastric cancer, squamous cell carcinoma, prostate cancer, and pancreatic cancer.

The cancer can be characterized by a solid tumor or a liquid tumor. In some embodiments, the cancer includes a solid tumor.

The cancer or solid tumor can be any cancer or solid tumor that responses to the treatment of a PTPN11 inhibitor. In some embodiments, the cancer or solid tumor is a tumor with one or more genes in MAPK pathway rearranged, mutated, or amplified. In some embodiments, the cancer or solid tumor is a tumor with one or more genes in MAPK pathway rearranged, mutated, or amplified, provided that the tumor is other than caused by a BRAF mutation including V600X mutation. In some embodiments, the solid tumor is a lung cancer, such as an advanced or metastatic non-small cell lung cancer (NSCLC). In some embodiments, the solid tumor is chordoma (also referred to as notochordal sarcoma).

In some embodiments, the cancer or solid tumor is a tumor characterized by a mutation in a RAS protein (e.g., KRAS, NRAS, or HRAS). In some embodiments, the cancer or solid tumor is characterized by a mutation in a RAS protein other than a Q61X mutation. In some embodiments, the cancer or solid tumor is characterized by a mutation in KRAS. In some embodiments, the cancer or solid tumor is characterized by a mutation in KRAS other than a KRAS Q61X mutation. In some embodiments, a KRAS protein comprises a G12C, G12D, G12S, G12V, G12R, G12A, G13D, G13A, G13C, G13R, G13S, and/or G13V mutation (e.g., the subject has a cancer characterized by a G12C, G12D, G12S, G12V, G12R, G12A, G13D, G13A, G13C, G13R, G13S, and/or G13V mutation in KRAS). In some embodiments, the cancer or solid tumor is characterized by a KRAS mutation including a KRAS G12C mutation. In some embodiments, the cancer or solid tumor is characterized by a KRAS mutation including a KRAS G12A mutation, a KRAS G12D mutation, a KRAS G12F mutation, a KRAS G12I mutation, a KRAS G12L mutation, a KRAS G12R mutation, a KRAS G12S mutation, a KRAS G12V mutation, a KRAS G12Y mutation, a KRAS G13D mutation, or a combination thereof.

In some embodiments, the cancer or solid tumor is a KRAS G12C-positive cancer or solid tumor. In some embodiments, the solid tumor is an advanced or metastatic KRAS G12C-positive cancer or solid tumor (e.g., lung cancer, colorectal cancer, pancreatic cancer, urothelial carcinoma, stomach cancer, mesothelioma, or a combination thereof). In some embodiments, the KRAS G12C-positive cancer or solid tumor is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma. In some embodiments, the cancer is small bowel cancer, appendiceal cancer, endometrial cancer, hepatobiliary cancer, small cell lung cancer, cervical cancer, germ cell tumor, ovarian cancer, gastrointestinal neuroendocrine tumor, bladder cancer, myelodysplastic/myeloproliferative neoplasms, head and neck cancer, esophagogastric cancer, soft tissue sarcoma, mesothelioma, thyroid cancer, leukemia, or melanoma. In some embodiments, the KRAS G12C-positive cancer or solid tumor is non-small cell lung cancer, colorectal cancer, pancreatic cancer, appendiceal cancer, endometrial cancer, cancer of unknown primary, ampullary cancer, gastric cancer, small bowel cancer, sinonasal cancer, bile duct cancer, or melanoma. In some embodiments, the solid tumor is an advanced or metastatic KRAS G12C-positive solid tumor selected from the group consisting of lung cancer, colorectal cancer, pancreatic cancer, urothelial carcinoma, stomach cancer, mesothelioma, and a combination thereof. In some embodiments, the solid tumor is an advanced or metastatic KRAS G12C-positive non-small cell lung cancer (NSCLC). In some embodiments, the solid tumor is an advanced or metastatic KRAS G12C-positive solid tumor, provided that the solid tumor is other than non-small cell lung cancer (NSCLC).

The cancer or solid tumor can also be any tumor that is resistant to the treatment of a KRAS G12C inhibitor (e.g., sotorasib (AMG 510), adagrasib (MRTX-849), MRTX1257, ARS-853, ARS-1620, JNJ-74699157 (ARS-3248), JDQ443, GDC-6036, JAB-21822, BI 1823911, MK-1084, LY3537982, and LY3499446). In some embodiments, the cancer or solid tumor is resistant to a KRAS G12C inhibitor. In some embodiments, the cancer or solid tumor is characterized by intrinsic and/or acquired resistance to a KRAS G12C inhibitor as defined and described herein. In some embodiments, the solid tumor is a KRAS G12C-positive solid tumor resistant to a KRAS G12C inhibitor. In some embodiments, the solid tumor is a KRAS G12C-positive solid tumor characterized by intrinsic and/or acquired resistance to a KRAS G12C inhibitor. In some embodiments, the solid tumor is resistant to the treatment of a KRAS G12C inhibitor selected from the group consisting of sotorasib (AMG 510), adagrasib (MRTX-849), MRTX1257, ARS-853, ARS-1620, JNJ-74699157 (ARS-3248), JDQ443, GDC-6036, JAB-21822, BI 1823911, MK-1084, LY3537982, and LY3499446. In some embodiments, the solid tumor is resistant to sotorasib (AMG 510). In some embodiments, the solid tumor is resistant to adagrasib (MRTX-849). In some embodiments, the solid tumor is a KRAS-positive solid tumor resistant to the treatment of a KRAS G12C inhibitor selected from the group consisting of sotorasib (AMG 510), adagrasib (MRTX-849), MRTX1257, ARS-853, ARS-1620, JNJ-74699157 (ARS-3248), JDQ443, GDC-6036, JAB-21822, BI 1823911, MK-1084, LY3537982, and LY3499446. In some embodiments, the solid tumor is a KRAS-positive solid tumor resistant to sotorasib (AMG 510). In some embodiments, the solid tumor is a KRAS-positive solid tumor resistant to adagrasib (MRTX-849). In some embodiments, the solid tumor is a KRAS G12C-positive solid tumor resistant to the treatment of a KRAS G12C inhibitor selected from the group consisting of sotorasib (AMG 510), adagrasib (MRTX-849), MRTX1257, ARS-853, ARS-1620, JNJ-74699157 (ARS-3248), JDQ443, GDC-6036, JAB-21822, BI 1823911, MK-1084, LY3537982, and LY3499446. In some embodiments, the solid tumor is a KRAS G12C-positive solid tumor resistant to sotorasib (AMG 510). In some embodiments, the solid tumor is a KRAS G12C-positive solid tumor resistant to adagrasib (MRTX-849).

In some embodiments, the cancer or solid tumor is a tumor in a subject who has one or more mutations in the MAPK pathway selected from the group consisting of one or mutations in NRAS, HRAS, CRAF, BRAF, NRAF, MAPK/ERK, MAPKK/MEK, NF1, IGFR, PDGFR, VEGFR, FGFR, CCKR, NGFR, EphR, AXLR, KEAP-1, TIE receptor, RYK receptor, DDR receptor, RET receptor, ROS receptor, LTK receptor, ROR receptor, MuSK receptor, a combination thereof. In some embodiments, the solid tumor is an advanced or metastatic solid tumor in a subject who has one or more mutations in MAPK pathway selected from the group consisting of one or more mutations in NRAS, HRAS, CRAF, BRAF, NRAF, MAPK/ERK, MAPKK/MEK, NF1, IGFR, PDGFR, VEGFR, FGFR, CCKR, NGFR, EphR, AXLR, TIE receptor, RYK receptor, DDR receptor, RET receptor, ROS receptor, LTK receptor, ROR receptor, MuSK receptor, and a combination thereof. In some embodiments, the solid tumor is an advanced or metastatic solid tumor in a subject who has a mutation in NRAS. In some embodiments, the solid tumor is an advanced or metastatic solid tumor in a subject who has a mutation in HRAS. In some embodiments, the subject has a mutation in CRAF. In some embodiments, the solid tumor is an advanced or metastatic solid tumor in a subject who has a mutation in NRAF. In some embodiments, the solid tumor is an advanced or metastatic solid tumor in a subject who has a mutation in MAPK/ERK. In some embodiments, the solid tumor is an advanced or metastatic solid tumor in a subject who has a mutation in MAPKK/MEK. In some embodiments, the solid tumor is an advanced or metastatic solid tumor in a subject who has a mutation in NF1. In some embodiments, the solid tumor is an advanced or metastatic solid tumor in a subject who has a mutation in IGFR. In some embodiments, the solid tumor is an advanced or metastatic solid tumor in a subject who has a mutation in PDGFR. In some embodiments, the solid tumor is an advanced or metastatic solid tumor in a subject who has a mutation in VEGFR. In some embodiments, the solid tumor is an advanced or metastatic solid tumor in a subject who has a mutation in FGFR. In some embodiments, the solid tumor is an advanced or metastatic solid tumor in a subject who has a mutation in CCKR. In some embodiments, the solid tumor is an advanced or metastatic solid tumor in a subject who has a mutation in NGFR. In some embodiments, the solid tumor is an advanced or metastatic solid tumor in a subject who has a mutation in EphR. In some embodiments, the solid tumor is an advanced or metastatic solid tumor in a subject who has a mutation in AXLR. In some embodiments, the solid tumor is an advanced or metastatic solid tumor in a subject who has a mutation in KEAP-1 receptor. In some embodiments, the solid tumor is an advanced or metastatic solid tumor in a subject who has a mutation in TIE receptor. In some embodiments, the solid tumor is an advanced or metastatic solid tumor in a subject who has a mutation in RYK receptor. In some embodiments, the solid tumor is an advanced or metastatic solid tumor in a subject who has a mutation in DDR receptor. In some embodiments, the subject has a mutation in RET receptor. In some embodiments, the solid tumor is an advanced or metastatic solid tumor in a subject who has a mutation in ROS receptor. In some embodiments, the solid tumor is an advanced or metastatic solid tumor in a subject who has a mutation in LTK receptor. In some embodiments, the solid tumor is an advanced or metastatic solid tumor in a subject who has a mutation in ROR receptor. In some embodiments, the solid tumor is an advanced or metastatic solid tumor in a subject who has a mutation in MuSK receptor.

In some embodiments, the solid tumor is an advanced or metastatic solid tumor in a subject who has a mutation in BRAF. In some embodiments, the solid tumor is an advanced or metastatic solid tumor in a subject who has a mutation in BRAF other than a V600X mutation. In some embodiments, the BRAF mutation(s) is class II (e.g., having intermediate to high kinase activity and RAS independence). In some embodiments, the BRAF mutation(s) is class III (e.g., having impaired kinase activity, upstream signaling dependence, and sensitivity to receptor tyrosine kinase (RTK) inhibitors). In some embodiments, the solid tumor is NSCLC characterized by a class II BRAF mutation (e.g., a mutation other than a V600X mutation). In some embodiments, the solid tumor is NSCLC characterized by a class III BRAF mutation (e.g., a mutation other than a V600X mutation).

The cancer or solid tumor can also be any tumor that is resistant to the treatment of an inhibitor (e.g., MEK inhibitors: cobimetinib, trametinib, binimetinib, mirdametinib, selumetinib; BRAF inhibitors: sorafenib, regorafenib, vemurafenib, encorafenib, dabrafenib) that targets, decreases, or inhibits the synthesis or biological activity in the MAPK pathway selected from the group consisting of one or more mutations in NRAS, HRAS, CRAF, BRAF, NRAF, MAPK/ERK, MAPKK/MEK, NF1, IGFR, PDGFR, VEGFR, FGFR, CCKR, NGFR, EphR, AXLR, KEAP-1, TIE receptor, RYK receptor, DDR receptor, RET receptor, ROS receptor, LTK receptor, ROR receptor, MuSK receptor, and a combination thereof. In some embodiments, the solid tumor is resistant to an inhibitor that targets, decreases, or inhibits the synthesis or biological activity in MAPK pathway selected from the group consisting of NRAS, HRAS, CRAF, BRAF, NRAF, MAPK/ERK, MAPKK/MEK, NF1, IGFR, PDGFR, VEGFR, FGFR, CCKR, NGFR, EphR, AXLR, KEAP-1, TIE receptor, RYK receptor, DDR receptor, RET receptor, ROS receptor, LTK receptor, ROR receptor, MuSK receptor, and a combination thereof. In some embodiments, the solid tumor is characterized by intrinsic and/or acquired resistance to an inhibitor that targets, decreases, or inhibits the synthesis or biological activity in MAPK pathway selected from the group consisting of NRAS, HRAS, CRAF, BRAF, NRAF, MAPK/ERK, MAPKK/MEK, NF1, IGFR, PDGFR, VEGFR, FGFR, CCKR, NGFR, EphR, AXLR, KEAP-1, TIE receptor, RYK receptor, DDR receptor, RET receptor, ROS receptor, LTK receptor, ROR receptor, MuSK receptor, and a combination thereof. In some embodiments, the solid tumor is a tumor in a subject who has one or more mutations in MAPK pathway resistant to an inhibitor that targets, decreases, or inhibits the synthesis or biological activity in MAPK pathway selected from the group consisting of NRAS, HRAS, CRAF, BRAF, NRAF, MAPK/ERK, MAPKK/MEK, NF1, IGFR, PDGFR, VEGFR, FGFR, CCKR, NGFR, EphR, AXLR, KEAP-1, TIE receptor, RYK receptor, DDR receptor, RET receptor, ROS receptor, LTK receptor, ROR receptor, MuSK receptor, and a combination thereof. In some embodiments, the solid tumor is a tumor in a subject who has one or more mutations in MAPK pathway characterized by intrinsic and/or acquired resistance to an inhibitor that targets, decreases, or inhibits the synthesis or biological activity in MAPK pathway selected from the group consisting of NRAS, HRAS, CRAF, BRAF, NRAF, MAPK/ERK, MAPKK/MEK, NF1, IGFR, PDGFR, VEGFR, FGFR, CCKR, NGFR, EphR, AXLR, KEAP-1, TIE receptor, RYK receptor, DDR receptor, RET receptor, ROS receptor, LTK receptor, ROR receptor, MuSK receptor, and a combination thereof.

In some embodiments, the solid tumor is an advanced or metastatic NF1 LOF solid tumor.

In some embodiments, the solid tumor is an advanced or metastatic BRAF class II/III mutant solid tumor. In some embodiments, the solid tumor is an advanced or metastatic BRAF class II mutant solid tumor. In some embodiments, the solid tumor is an advanced or metastatic BRAF class III mutant solid tumor.

In some embodiments, the cancer or solid tumor is sarcomas. In some embodiments, the cancer or tumor is chordoma or notochord sarcoma.

In some embodiments, the solid tumor is an EGFR-positive solid tumor. In some embodiments, the solid tumor is an advanced or metastatic EGFR-positive solid tumor (e.g., biliary tract cancer, brain cancer, breast cancer, cervical cancer, colorectal cancer, esophageal cancer, gastric cancer, head and neck squamous cell carcinoma (HNSCC), lung cancer, pancreatic cancer, thyroid cancer, or a combination thereof). In some embodiments, the solid tumor is an advanced or metastatic EGFR-positive non-small cell lung cancer (NSCLC). In some embodiments, the solid tumor is an advanced or metastatic EGFR-positive non-small cell lung cancer (NSCLC), optionally progressed on a standard of care EGFR TKI therapy. In some embodiments, the solid tumor is an advanced or metastatic EGFR-positive non-small cell lung cancer (NSCLC), progressed on a standard of care EGFR TKI therapy.

The solid tumor can also be any tumor that is resistant to the treatment of an EGFR inhibitor (e.g., a selective EGFR inhibitor or an EGFR/HER2 dual inhibitor). In some embodiments, the solid tumor is resistant to an EGFR inhibitor (e.g., erlotinib, cetuximab, panitumumab, vandetanib, afatinib, gefitinib, osimertinib, necitumumab, brigatinib, neratinib, dacomitinib, amivantamab (JNJ-61186372), mobocertinib (TAK-788), BLU-945, varlitinib, tarloxitinib, poziotinib, or lapatinib). In some embodiments, the solid tumor is characterized by intrinsic and/or acquired resistance to an EGFR inhibitor. In some embodiments, the solid tumor is an EGFR-positive solid tumor resistant to an EGFR inhibitor. In some embodiments, the solid tumor is an EGFR-positive solid tumor characterized by intrinsic and/or acquired resistance to an EGFR inhibitor. In some embodiments, the solid tumor is characterized by EGFR-dependent and/or EGFR-independent resistance to an EGFR inhibitor.

In any one of embodiments, a standard of care or curative therapy is unavailable for treating the solid tumor, as described herein.

### III-4: Treatment Cycle and Dose Adjustment

Treatment with the compound of formula (I) or (10b) can include one or more treatment cycles (e.g., 1 to 6 treatments, such as at least 1, 2, 3, 4, 5, 6, or more treatment cycles). In some embodiments, the treatment includes one or more treatment cycles (e.g., 1 to 6 treatments, such as at least 1, 2, 3, 4, 5, 6, or more treatment cycles). In some embodiments, the treatment includes at least 2, 3, 4, 5, 6, or more treatment cycles. In some embodiments, the treatment includes 2 to 6 treatment cycles. In some embodiments, the treatment includes 3 to 6 treatment cycles. In some embodiments, the treatment includes 4 to 6 treatment cycles. In some embodiments, the treatment includes 5 to 6 treatment cycles. In some embodiments, the treatment includes 6 treatment cycles.

After a previous treatment cycle, a dose of the compound of formula (I) or (10b) can be adjusted (e.g., dose escalation or de-escalation). Dose adjustment may be based at least in part on a safety evaluation (e.g., a dose-limiting toxicity (DLT) assessment). In some embodiments, the administration of the compound of formula (I) or (10b) includes a dose escalation or de-escalation after a previous treatment cycle, wherein the dose escalation or de-escalation is determined by a dose-limiting toxicity (DLT) assessment. In some embodiments, the administration of the compound of formula (I) or (10b) includes a dose escalation after a previous treatment cycle, if safety assessment meets accepted criteria of Example 7. In some embodiments, the administration of the compound of formula (I) or (10b) includes a dose escalation in a second treatment after a first treatment cycle if safety assessment meets accepted criteria of Example 7. In some embodiments, the administration of the compound of formula (I) or (10b) includes a dose escalation in a third treatment after a second treatment cycle if safety assessment meets accepted criteria of Example 7. In some embodiments, the administration of the compound of formula (I) or (10b) includes a dose escalation in a fourth treatment after a third treatment cycle if safety assessment meets accepted criteria of Example 7. In some embodiments, the administration of the compound of formula (I) or (10b) includes a dose escalation in a fifth treatment after a fourth treatment cycle if safety assessment meets accepted criteria of Example 7. In some embodiments, the administration of the compound of formula (I) or (10b) includes a dose escalation in a sixth treatment after a fifth treatment cycle if safety assessment meets accepted criteria of Example 7. In some embodiments, the administration of the compound of formula (I) or (10b) includes a dose de-escalation in a third treatment after a second treatment cycle according to the criteria of Example 7. In some embodiments, the administration of the compound of formula (I) or (10b) includes a dose de-escalation in a fourth treatment after a third treatment cycle according to the criteria of Example 7. In some embodiments, the administration of the compound of formula (I) or (10b) includes a dose de-escalation in a fifth treatment after a fourth treatment cycle according to the criteria of Example 7. In some embodiments, the administration of the compound of formula (I) or (10b) includes a dose de-escalation in a sixth treatment after a fifth treatment cycle according to the criteria of Example 7. In some embodiments, the administration of the compound of formula (I) or (10b) includes a dose de-escalation within a treatment cycle, according to the criteria of Example 7.

In some embodiments, the administration of the compound of formula (I) or (10b) includes 1 to 6 dose escalations, optionally 1 to 2 dose de-escalations. In some embodiments, the administration of the compound of formula (I) or (10b) includes 1 to 6 dose escalations. In some embodiments, the administration of the compound of formula (I) or (10b) includes 1 to 5 dose escalations. In some embodiments, the administration of the compound of formula (I) or (10b) includes 2 to 5 dose escalations. In some embodiments, the administration of the compound of formula (I) or (10b) includes 3 to 5 dose escalations. In some embodiments, the administration of the compound of formula (I) or (10b) includes 4 to 5 dose escalations. In some embodiments, the administration of the compound of formula (I) or (10b) includes 5 dose escalations. In some embodiments, the administration of the compound of formula (I) or (10b) includes 1 to 2 dose de-escalations. In some embodiments, the administration of the compound of formula (I) or (10b) includes one (1) dose de-escalations.

In some embodiments, each of one or more treatment cycles has a duration of about 28 days and the compound of formula (I) or (10b) is administered daily. In some embodiments, the first treatment cycle has a duration of about 28 days and the compound of formula (I) or (10b) is administered daily. In some embodiments, the second treatment cycle has a duration of about 28 days and the compound of formula (I) or (10b) is administered daily. In some embodiments, the third treatment cycle has a duration of about 28 days and the compound of formula (I) or (10b) is administered daily. In some embodiments, the fourth treatment cycle has a duration of about 28 days and the compound of formula (I) or (10b) is administered daily. In some embodiments, the fifth treatment cycle has a duration of about 28 days and the compound of formula (I) or (10b) is administered daily. In some embodiments, the six treatment cycle has a duration of about 28 days and the compound of formula (I) or (10b) is administered daily.

### III-5: Therapeutically Effective Amount/Administration

In some embodiments, the therapeutically effective amount is a total daily dosage of no more than about 2000 mg of the compound of formula (I) or (10b) on a salt-free and anhydrous basis. In some embodiments, the therapeutically effective amount is a total daily dosage of from about 10 mg to about 2000 mg, from about 50 mg to about 2000 mg, from about 80 mg to about 2000 mg, from about 80 mg to about 1000 mg, from about 80 mg to about 700 mg, from about 80 mg to about 550 mg, from about 80 mg to about 400 mg, from about 80 mg to about 250 mg, or from about 80 mg to about 150 mg of the compound of formula (I) or (10b), on a salt-free and anhydrous basis, or any useful range therein. In some embodiments, the therapeutically effective amount is a total daily dosage of from about 80 mg to about 700 mg, from about 80 mg to about 550 mg, from about 80 mg to about 450 mg, from about 80 mg to about 400 mg, from about 80 mg to about 250 mg, or from about 80 mg to about 150 mg of the compound of formula (I) or (10b), on a salt-free and anhydrous basis, or any useful range therein.

In some embodiments, the therapeutically effective amount is a total daily dosage of no more than about 2000 mg of the compound of formula (10b) on a salt-free and anhydrous basis. In some embodiments, the therapeutically effective amount is a total daily dosage of from about 10 mg to about 2000 mg, from about 50 mg to about 2000 mg, from about 80 mg to about 2000 mg, from about 80 mg to about 1000 mg, from about 80 mg to about 700 mg, from about 80 mg to about 550 mg, from about 80 mg to about 400 mg, from about 80 mg to about 250 mg, or from about 80 mg to about 150 mg of the compound of formula (10b), on a salt-free and anhydrous basis, or any useful range therein. In some embodiments, the therapeutically effective amount is a total daily dosage of from about 80 mg to about 700 mg, from about 80 mg to about 550 mg, from about 80 mg to about 450 mg, from about 80 mg to about 400 mg, from about 80 mg to about 250 mg, or from about 80 mg to about 150 mg of the compound of formula (10b), on a salt-free and anhydrous basis, or any useful range therein. In some embodiments, the therapeutically effective amount is a total daily dosage of about 80 mg, about 150 mg, about 250 mg, about 400 mg, about 450 mg, about 550 mg, or about 700 mg of the compound of formula (10b), on a salt-free and anhydrous basis. In some embodiments, the therapeutically effective amount is a total daily dosage of about 80 mg of the compound of formula (10b), on a salt-free and anhydrous basis. In some embodiments, the therapeutically effective amount is a total daily dosage of about 150 mg of the compound of formula (10b), on a salt-free and anhydrous basis. In some embodiments, the therapeutically effective amount is a total daily dosage of about 250 mg of the compound of formula (10b), on a salt-free and anhydrous basis. In some embodiments, the therapeutically effective amount is a total daily dosage of about 400 mg of the compound of formula (10b), on a salt-free and anhydrous basis. In some embodiments, the therapeutically effective amount is a total daily dosage of about 450 mg of the compound of formula (10b), on a salt-free and anhydrous basis. In some embodiments, the therapeutically effective amount is a total daily dosage of about 550 mg of the compound of formula (10b), on a salt-free and anhydrous basis. In some embodiments, the therapeutically effective amount is a total daily dosage of about 700 mg of the compound of formula (10b), on a salt-free and anhydrous basis.

In general, the compound of formula (I) or (10b) can be administered orally. In some embodiments, the compound of formula (I) or (10b) is administered orally. In some embodiments, the compound of formula (I) is administered orally. In some embodiments, the compound of formula (10b) is administered orally. In some embodiments, the compound of formula (I) in a tablet formulation is administered orally. In some embodiments, the compound of formula (10b) in a tablet formulation is administered orally.

In general, the compound of formula (I) or (10b) can be administered once or multiple times (e.g., 2, 3, 4, or more times) daily. In some embodiments, the compound of formula (I) or (10b) is administered once, twice, three times, or four times daily. In some embodiments, the compound of formula (I) or (10b) is administered once daily. In some embodiments, the compound of formula (I) or (10b) is administered twice daily. In some embodiments, the compound of formula (10b) is administered once, twice, three times, or four times daily. In some embodiments, the compound of formula (10b) is administered once daily. In some embodiments, the compound of formula (10b) is administered twice daily.

The compound of formula (I) or (10b) can be in an oral dosage form in one or more dosage strengths, where the compound of formula (I) or (10b) is present in an amount of at least about 1 mg, 5 mg, 10 mg, 20 mg, 30 mg, 50 mg, 90 mg, 100 mg, 120 mg, 180 mg, 200 mg, 300 mg, 400 mg, or 500 mg, on a salt-free and anhydrous basis. In some embodiments, the oral dosage form is a tablet formulation in one or more dosage strengths. In some embodiments of the tablet formulation, the compound of formula (I) or (10b) is present in an amount of from 1 to 1000 mg, from 1 to 750 mg, from 1 to 500 mg, from 1 to 250 mg, from 30 to 1000 mg, from 30 to 750 mg, from 30 to 500 mg, from 30 to 200 mg, from 30 to 180 mg, from 30 to 120 mg, from 30 to 90 mg, from 50 to 1000 mg, from 50 to 750 mg, from 50 to 500 mg, from 50 to 250 mg, from 100 to 1000 mg, from 100 to 750 mg, from 100 to 500 mg, from 100 to 250 mg, from 200 to 1000 mg, from 200 to 750 mg, from 200 to 500 mg, from 300 to 1000 mg, from 300 to 750 mg, from 300 to 500 mg, from 400 to 1000 mg, from 400 to 750 mg, from 500 to 1000 mg, from 500 to 750 mg, from 600 to 1000 mg, from 5 to 250 mg, or from 5 to 100 mg in each tablet, on a salt-free and anhydrous basis. In some embodiments of the tablet formulation, the compound of formula (I) or (10b) is present in an amount of about 5 mg, 10 mg, 30 mg, 50 mg, 100 mg, 150 mg, 200 mg, 250 mg, 300 mg, 400 mg, 500 mg, 600 mg, 700 mg, 800 mg, 900 mg, or 1000 mg in each tablet, on a salt-free and anhydrous basis. In some embodiments of the tablet formulation, the compound of formula (I) or (10b) is present in an amount of about 30 mg, 50 mg, or 100 mg in each tablet, on a salt-free and anhydrous basis.

The compound of formula (10b) can be in an oral dosage form in one or more dosage strengths, where the compound of formula (10b) is present in an amount of at least about 1 mg, 5 mg, 10 mg, 20 mg, 30 mg, 50 mg, 90 mg, 100 mg, 120 mg, 180 mg, 200 mg, 300 mg, 400 mg, or 500 mg, on a salt-free and anhydrous basis. In some embodiments, the oral dosage form is a tablet formulation in one or more dosage strengths. In some embodiments of the tablet formulation, the compound of formula (10b) is present in an amount of from 1 to 1000 mg, from 1 to 750 mg, from 1 to 500 mg, from 1 to 250 mg, from 30 to 1000 mg, from 30 to 750 mg, from 30 to 500 mg, from 30 to 200 mg, from 30 to 180 mg, from 30 to 120 mg, from 30 to 90 mg, from 50 to 1000 mg, from 50 to 750 mg, from 50 to 500 mg, from 50 to 250 mg, from 100 to 1000 mg, from 100 to 750 mg, from 100 to 500 mg, from 100 to 250 mg, from 200 to 1000 mg, from 200 to 750 mg, from 200 to 500 mg, from 300 to 1000 mg, from 300 to 750 mg, from 300 to 500 mg, from 400 to 1000 mg, from 400 to 750 mg, from 500 to 1000 mg, from 500 to 750 mg, from 600 to 1000 mg, from 5 to 250 mg, or from 5 to 100 mg in each tablet, on a salt-free and anhydrous basis. In some embodiments of the tablet formulation, the compound of formula (10b) is present in an amount of about 5 mg, 10 mg, 30 mg, 50 mg, 100 mg, 150 mg, 200 mg, 250 mg, 300 mg, 400 mg, 500 mg, 600 mg, 700 mg, 800 mg, 900 mg, or 1000 mg in each tablet, on a salt-free and anhydrous basis. In some embodiments of the tablet formulation, the compound of formula (10b) is present in an amount of about 30 mg, 50 mg, or 100 mg in each tablet, on a salt-free and anhydrous basis. In some embodiments of the tablet formulation, the compound of formula (10b) is present in an amount of about 30 mg in each tablet, on a salt-free and anhydrous basis. In some embodiments of the tablet formulation, the compound of formula (10b) is present in an amount of about 50 mg in each tablet, on a salt-free and anhydrous basis. In some embodiments of the tablet formulation, the compound of formula (10b) is present in an amount of about 100 mg in each tablet, on a salt-free and anhydrous basis.

In some embodiments, the compound of formula (10b) is administered once daily to provide a total daily dosage of no more than about 2000 mg of the compound of formula (10b). In some embodiments, the compound of formula (10b) is administered once daily to provide a total daily dosage of from about 80 mg to about 700 mg, from about 80 mg to about 550 mg, from about 80 mg to about 450 mg, from about 80 mg to about 400 mg, from about 80 mg to about 250 mg, or from about 80 mg to about 150 mg of the compound of formula (10b), on a salt-free and anhydrous basis. In some embodiments, the compound of formula (10b) is administered once daily to provide a total daily dosage of about 80 mg, about 150 mg, about 250 mg, about 400 mg, about 450 mg, about 550 mg, or about 700 mg of the compound of formula (10b), on a salt-free and anhydrous basis. In some embodiments, the compound of formula (10b) is administered once daily to provide a total daily dosage of about 450 mg of the compound of formula (10b), on a salt-free and anhydrous basis.

In some embodiments, the compound of formula (10b) is administered once daily during each of one or more treatment cycles, as described herein. In some embodiments, the compound of formula (10b) is administered once daily to provide a total daily dosage of about 450 mg of the compound of formula (10b), on a salt-free and anhydrous basis, during each of one or more treatment cycles, as described herein.

In general, the compound of formula (I) or (10b) is recommend to be administered to a subject without food (e.g., after an overnight fast (minimum 8 hours) followed by 2 hours of fasting after the dose is taken). The subject is allowed to have water except for one (1) hour before and after the administration and the subject is given with water (e.g., 240 mL) at the administration. In some embodiments, the compound of formula (I) or (10b) is administered to the subject without food, at least about 8 hours prior to the administration and at least about 2 hours post the administration. In some embodiments, the compound of formula (10b) is administered to the subject without food, at least about 8 hours prior to the administration and at least about 2 hours post the administration. In some embodiments, the compound of formula (10b) is administered to the subject with food and/or without fasting.

### III-6: Efficacy

A Phase 1/1B, first-in-human study can evaluate the safety, tolerability, and efficacy of the compound of formula (10b) to reduces or stabilize solid tumors in subjects, as summarized in Example 7.

Administration of a therapeutically effective amount of the compound of formula (I) or (10b) can reduce or substantially eliminate solid tumors in subjects. In some embodiments, the therapeutically effective amount of formula (I) or (10b) substantially eliminates the solid tumor. In some embodiments, the therapeutically effective amount of formula (I) or (10b) reduces a volume of the solid tumor at least about 10%, about 20%, about 30%, about 40%, about 50%, about 60%, about 70%, about 80%, about 90%, or more. In some embodiments, the therapeutically effective amount of formula (I) or (10b) reduces a volume of the solid tumor in a size of from about 10% to about 90%, from about 10% to about 80%, from about 10% to about 70%, from about 10% to about 60%, from about 10% to about 50%, from about 10% to about 40%, from about 10% to about 30%, from about 10% to about 20%, from about 20% to about 90%, from about 20% to about 80%, from about 20% to about 70%, from about 20% to about 60%, from about 20% to about 50%, from about 20% to about 40%, from about 20% to about 30%, from about 30% to about 90%, from about 30% to about 80%, from about 30% to about 70%, from about 30% to about 60%, from about 30% to about 50%, from about 30% to about 40%, from about 40% to about 90%, from about 40% to about 80%, from about 40% to about 70%, from about 40% to about 60%, from about 40% to about 50%, from about 50% to about 90%, from about 50% to about 80%, from about 50% to about 70%, from about 50% to about 60%, from about 60% to about 90%, from about 60% to about 80%, from about 60% to about 70%, from about 70% to about 90%, from about 70% to about 80%, from about 80% to about 90%, or any range therein. In some embodiments, the therapeutically effective amount of formula (I) or (10b) reduces a volume of the solid tumor about 10%, about 20%, about 30%, about 40%, about 50%, about 60%, about 70%, about 80%, or about 90%. In some embodiments, the therapeutically effective amount of formula (10b) reduces a volume of the solid tumor about 10%, about 20%, about 30%, about 40%, about 50%, about 60%, about 70%, about 80%, or about 90%, wherein the compound of formula (10b) is administered once daily to provide a total daily dosage of about 450 mg of the compound of formula (10b), on a salt-free and anhydrous basis.

Administration of a therapeutically effective amount of the compound of formula (I) or (10b) can stabilize solid tumors in subjects. In some embodiments, the therapeutically effective amount of formula (I) or (10b) stabilize the solid tumor. In some embodiments, the therapeutically effective amount of formula (10b) stabilize the solid tumor, wherein the compound of formula (10b) is administered once daily to provide a total daily dosage of about 450 mg of the compound of formula (10b), on a salt-free and anhydrous basis.

Administration of a therapeutically effective amount of the compound of formula (I) or (10b) can maintain a reduction or stabilization of solid tumors in subjects for a period of time (e.g., 1 to 12 months). In some embodiments, the solid tumor is reduced or stabilized for a period of at least about one month with the therapeutically effective amount of the compound of formula (I) or (10b). In some embodiments, the solid tumor is reduced or stabilized for a period of at least about 2, 3, 4, 5, 6, 7, 8, 9, 10, 11, or 12 months with the therapeutically effective amount of the compound of formula (I) or (10b). In some embodiments, the solid tumor is reduced or stabilized for a period of from about 1 to about 12 months, from about 1 to about 6 months, from about 1 to about 3 months, or from about 1 to about 2 months.

In some embodiments, the subject is further evaluated to by one or more tests (e.g., tests according to Table 3, Table 4, and Table 5) to provide overall assessments including plasma pharmacokinetic and/or pharmacodynamic profiles. Examples of such tests are described in, e.g., Table 3, Table 4, and Table 5 of Example 7.

In some embodiments, the subject is further evaluated for one or more biomarkers to determine a correlation of the one or more biomarkers to an antitumor response. Examples of such evaluation are described in Table 3, Table 4, and Table 5 of Example 7.

### III-7: Oral Dosage Form

The oral dosage form including the compound of formula (I) or (10b) can be in any oral dosage forms including one or more pharmaceutically acceptable carriers and/or excipients. Oral preparations include tablets, pills, powder, dragees, capsules, liquids, lozenges, cachets, gels, syrups, slurries, suspensions, etc., suitable for ingestion by the patient.

For preparing oral dosage forms including the compound of formula (I) or (10b), pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired.

The powders, capsules and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the dosage forms can also be used orally using, for example, push-fit capsules made of gelatin, as well as soft, sealed capsules made of gelatin and a coating such as glycerol or sorbitol. Push-fit capsules can contain the compound of formula (I) or (10b) mixed with a filler or binders such as lactose or starches, lubricants such as talc or magnesium stearate, and, optionally, stabilizers. In soft capsules, the compound of formula (I) or (10b) may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compound of formula (I) or (10b) are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions.

Aqueous solutions suitable for oral use can be prepared by dissolving the compound of formula (I) or (10b) in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of formula (I) or (10b) in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations including the compound of formula (I) or (10b) can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

### III-8: Embodiments

In one aspect, the present disclosure provides a method of treating a tumor, the method including administering to a subject in need thereof a therapeutically effective amount of a compound represented by formula (10b):

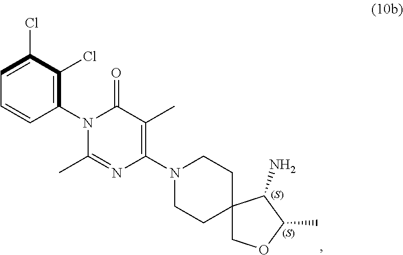

or a tautomer thereof, wherein the subject has (i) one or more mutations in MAPK pathway, provided that the one or more mutations in MAPK pathway are other than a BRAF mutation comprising V600X mutation, and/or (ii) one or more mutations in PTPN11.

In some embodiments, the present disclosure provides a method of treating an advanced or metastatic KRAS G12C-positive solid tumor, the method including administering to a subject in need thereof a therapeutically effective amount of a compound represented by formula (10b):

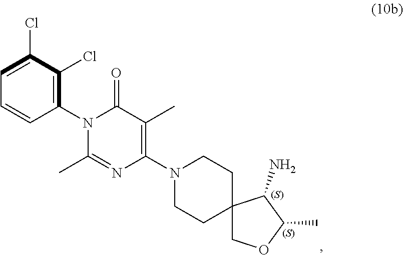

or a tautomer thereof, wherein the subject has (i) one or more mutations in MAPK pathway, provided that the one or more mutations in MAPK pathway are other than a BRAF mutation comprising V600X mutation, and/or (ii) one or more mutations in PTPN11.

In some embodiments, the present disclosure provides a method of treating an advanced or metastatic non-small cell lung cancer (NSCLC), the method including administering to a subject in need thereof a therapeutically effective amount of a compound represented by formula (10b):

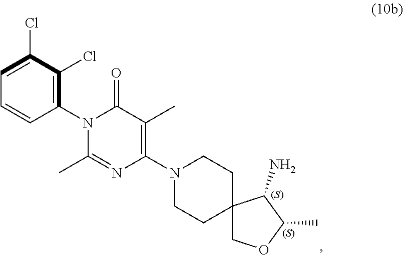

or a tautomer thereof, wherein the subject has (i) one or more mutations in MAPK pathway, provided that the one or more mutations in MAPK pathway are other than a BRAF mutation comprising V600X mutation, and/or (ii) one or more mutations in PTPN11.

In some embodiments, the present disclosure provides a method of treating an advanced or metastatic KRAS G12C-positive non-small cell lung cancer (NSCLC), the method including administering to a subject in need thereof a therapeutically effective amount of a compound represented by formula (10b):

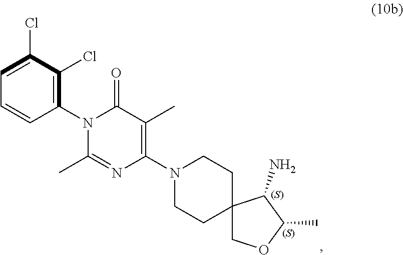

or a tautomer thereof, wherein the subject has (i) one or more mutations in MAPK pathway, provided that the one or more mutations in MAPK pathway are other than a BRAF mutation comprising V600X mutation, and/or (ii) one or more mutations in PTPN11.

In some embodiments, the present disclosure provides a method of treating an advanced or metastatic KRAS G12C-positive solid tumor, provided that the solid tumor is other than non-small cell lung cancer (NSCLC), the method including administering to a subject in need thereof a therapeutically effective amount of a compound represented by formula (10b):

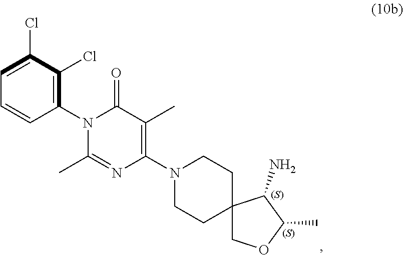

or a tautomer thereof, wherein the subject has (i) one or more mutations in MAPK pathway, provided that the one or more mutations in MAPK pathway are other than a BRAF mutation comprising V600X mutation, and/or (ii) one or more mutations in PTPN11.

In some embodiments, the present disclosure provides a method of treating an advanced or metastatic NF1 LOF solid tumor, the method including administering to a subject in need thereof a therapeutically effective amount of a compound represented by formula (10b):

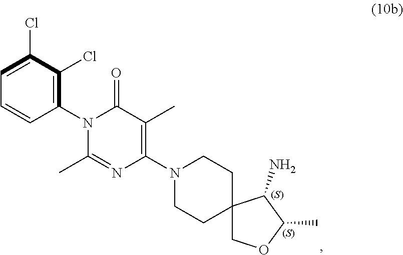

or a tautomer thereof, wherein the subject has (i) one or more mutations in MAPK pathway, provided that the one or more mutations in MAPK pathway are other than a BRAF mutation comprising V600X mutation, and/or (ii) one or more mutations in PTPN11.

In some embodiments, the present disclosure provides a method of treating an advanced or metastatic EGFR-positive non-small cell lung cancer (NSCLC), optionally progressed on a standard of care EGFR tyrosine kinase inhibitor (TKI) therapy, the method including administering to a subject in need thereof a therapeutically effective amount of a compound represented by formula (10b):

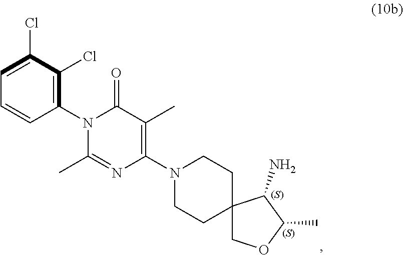

or a tautomer thereof, wherein the subject has (i) one or more mutations in MAPK pathway, provided that the one or more mutations in MAPK pathway are other than a BRAF mutation comprising V600X mutation, and/or (ii) one or more mutations in PTPN11.

In some embodiments, the present disclosure provides a method of treating an advanced or metastatic BRAF class II/II mutant solid tumor, the method including administering to a subject in need thereof a therapeutically effective amount of a compound represented by formula (10b):

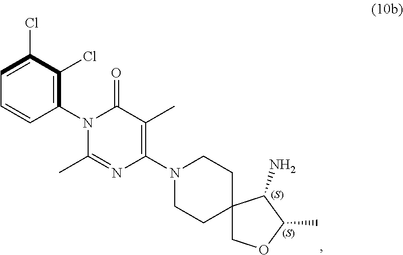

or a tautomer thereof, wherein the subject has (i) one or more mutations in MAPK pathway, provided that the one or more mutations in MAPK pathway are other than a BRAF mutation comprising V600X mutation, and/or (ii) one or more mutations in PTPN11.

In some embodiments, the present disclosure provides a method of treating chordoma, the method including administering to a subject in need thereof a therapeutically effective amount of a compound represented by formula (10b):

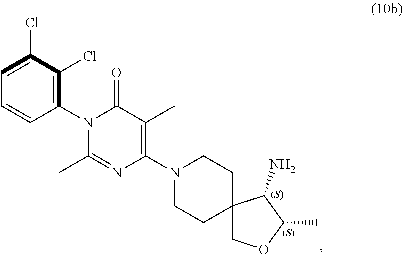

or a tautomer thereof, wherein the subject has (i) one or more mutations in MAPK pathway, provided that the one or more mutations in MAPK pathway are other than a BRAF mutation comprising V600X mutation, and/or (ii) one or more mutations in PTPN11.

The compound of formula (10b) is described according to Section III-1: Compound of Formula (I). In some embodiments, the compound of formula (10b) is any one of embodiments as described in Section III-1.

The subject is described according to Section III-2: Subject. In some embodiments, the subject is any one of embodiments as described in Section III-3: Subject. In some embodiments, the subject has a KRAS G12C mutation.

The solid tumor is described according to Section III-3: Cancer/Solid Tumor. In some embodiments, the solid tumor is any one of embodiments as described in Section III-2:

Cancer/Solid Tumor. In some embodiments, the solid tumor is an advanced or metastatic KRAS G12C-positive non-small cell lung cancer (NSCLC). In some embodiments, the solid tumor is an advanced or metastatic KRAS G12C-positive solid tumor, provided that the solid tumor is other than non-small cell lung cancer (NSCLC). In some embodiments, In some embodiments, the solid tumor is an advanced or metastatic NF1 LOF solid tumor. In some embodiments, the solid tumor is an advanced or metastatic EGFR-positive non-small cell lung cancer (NSCLC), optionally progressed on a standard of care EGFR tyrosine kinase inhibitor (TKI) therapy. In some embodiments, the solid tumor is an advanced or metastatic BRAF class II/II mutant solid tumor. In some embodiments, the cancer or solid tumor is chordoma or notochord sarcoma.

The treatment cycle and dose adjustment are described according to Section III-4: Treatment Cycle and Dose Adjustment. In some embodiments, the treatment cycle and dose adjustment are any one of embodiments as described in Section III-4: Treatment Cycle and Dose Adjustment.

The therapeutically effective amount and/or administration of formula (10b) are described according to Section III-5: Therapeutically Effective Amount/Administration. In some embodiments, the therapeutically effective amount and/or administration of formula (10b) are any one of embodiments as described in Section III-5: Therapeutically Effective Amount/Administration. In some embodiments, the therapeutically effective amount is a total daily dosage of about 450 mg of the compound of formula (10b), on a salt-free and anhydrous basis. In some embodiments, the compound of formula (10b) is administered once daily to provide a total daily dosage of about 450 mg of the compound of formula (10b), on a salt-free and anhydrous basis. In some embodiments, the compound of formula (10b) is administered once daily orally. In some embodiments, the compound of formula (10b) in a total daily dosage of about 450 mg is administered once daily orally.

Efficacy is described according to Section III-6: Efficacy. In some embodiments, he therapeutically effective amount and/or administration are any one of embodiments as described in Section III-6: Efficacy. In some embodiments, the therapeutically effective amount of formula (10b) reduces a volume of the solid tumor about 10%, about 20%, about 30%, about 40%, about 50%, about 60%, about 70%, about 80%, or about 90%, wherein the compound of formula (10b) is administered once daily to provide a total daily dosage of about 450 mg of the compound of formula (10b), on a salt-free and anhydrous basis. In some embodiments, the therapeutically effective amount of formula (10b) stabilize the solid tumor, wherein the compound of formula (10b) is administered once daily to provide a total daily dosage of about 450 mg of the compound of formula (10b), on a salt-free and anhydrous basis.

### IV. Kits

In another aspect, the present disclosure provides a kit for treating cancer or a solid tumor in a subject, the kit including a therapeutically effective amount of a compound represented by formula (I) together with instruction for effective administration, wherein the compound of formula (I) and the subject are each as defined and described herein.

The subject is described according to Section III-2: Subject. In some embodiments, the subject is any of embodiments as described in Section III-2: Subject.

The cancer and/or solid tumor are described according to Section III-3: Cancer/Solid Tumor. In some embodiments, the cancer and/or solid tumor are any of embodiments as described in Section III-3: Cancer/Solid Tumor.

The compound of formula (I) is described according to Section III-1: Compound of Formula (I). In some embodiments, the compound of formula (I) is any of embodiments as described in Section III-1: Compound of Formula (I). In some embodiments, the compound of formula (I) is the compound of formula (10b).

In some embodiments, the kit includes instructions for administration of the compound of formula (I) or (10b). In some embodiments, the kit includes instructions for administration of the compound of formula (10b). In some embodiments, such instructions include directions relating to safety provisions as well as timing and amounts of administration of the compound of formula (I) or (10b). In some embodiments, such instructions include directions relating to safety provisions as well as timing and amounts of administration of the compound of formula (10b).

V. LIST OF ABBREVIATIONS

| Abbreviation | Definition |
|---|---|
| AE | Adverse event |
| ALK | Anaplastic Lymphoma Kinase |
| ALT | Alanine aminotransferase (or transaminase) |
| ANC | Absolute neutrophil count |
| aPTT | Activated partial thromboplastin time |
| AST | Aspartate aminotransferase (or transaminase) |
| AUC | Area under the curve |
| BBP | BridgeBio Pharma |
| BOIN | Bayesian optimal interval design |
| BP | Blood pressure |
| CBC | Complete blood count |
| cfDNA | Circulating free deoxyribonucleic acid |
| CFR | Code of Federal Regulations |
| Cₘₐₓ | Maximum plasma drug concentration |
| CNS | Central nervous system |
| CO₂ | Carbon dioxide |
| CPK | Creatinine phosphokinase |
| CR | Complete response(s) |
| CT | Computed tomography |
| CTCAE | Common Terminology Criteria for Adverse Events |
| CYP | Cytochrome P450 |
| DLT | Dose-limiting toxicity |
| DOR | Duration of response |
| ECG | Electrocardiogram |
| ECOG | Eastern Cooperative Oncology Group |
| EDC | Electronic data capture (system) |
| EGFR | Epidermal Growth Factor Receptor |
| EOS | End of study |
| ERK | Extracellular signal-regulated kinase |
| FAS | Full analysis set |
| FDA | Food and Drug Administration |
| FISH | Fluorescence in situ hybridization |
| GCP | Good Clinical Practice |
| GLP | Good Laboratory Practice |
| HBV | Hepatitis B virus |
| HCV | Hepatitis C virus |
| HIV | Human immunodeficiency virus |
| HNSTD | Highest Non-Severely Toxic Dose |
| HR | Heart rate |
| IACS | Institute for Applied Cancer Science |
| IB | Investigator's Brochure |
| ICH | International Conference on Harmonization |
| IEC | Independent ethics committee |
| IHC | Immunohistochemistry |
| IND | Investigational New Drug application |
| INR | International normalized ratio |
| IRB | Institutional review board |
| KRAS | Kirsten Rat Sarcoma Viral Oncogene homolog |
| LD (or LDi) | Longest diameter |
| LOF | Loss-of-function |
| LVEF | Left ventricular ejection fraction |
| MAPK | Mitogen-Activated Protein Kinase |
| MDACC | The University of Texas MD Anderson Cancer Center |
| MedDRA | Medical Dictionary for Regulatory Activities |
| MEK | Mitogen-activated protein kinase |
| MRI | Magnetic resonance imaging |
| MTD | Maximum tolerated dose |
| MUGA | Multiple-gated acquisition scan (radionuclide angiography) |
| NCI | National Cancer Institute |
| NOAEL | No observed adverse effect level |
| NSCLC | Non-Small Cell Lung Cancer |
| ORR | Objective response rate |
| PD | Progressive disease |
| PET | Positron emission tomography |
| PFS | Progression free survival |
| P-gp | P-glycoprotein |
| PK | Pharmacokinetic |
| PR | Partial response |
| PT | Prothrombin time |
| PTPN11 | protein tyrosine phosphatase non-receptor type 11, the gene encoding SHP2 |
| PTT | Partial thromboplastin time |
| QD | Once daily |
| RP2D | Recommended phase 2 dose |
| RR | Respiratory rate |
| RTK | Receptor Tyrosine Kinase |
| RVO | Retinal vein occlusion |
| SAE | Serious adverse event |
| SD | Stable disease or standard deviation |
| SGOT | Serum glutamic-oxaloacetic transaminase |
| SGPT | Serum glutamic-pyruvic transaminase |
| SHP2 | Src Homology 2 domain-containing protein tyrosine Phosphatase 2 |
| SRC | Safety Review Committee |
| STD10 | Severely Toxic Dose in 10% of the animals |
| SUSAR | Suspected Unexpected Serious Adverse Reaction |
| TEAE | Treatment-emergent adverse event |
| Tₘₐₓ | Time of maximum plasma concentration following drug administration |
| ULN | Upper limit of normal |
| US | Ultrasound |
| USA | United States of America |
| WOCBP | Women of childbearing potential |

### VI. Examples

### Example 1: In Vitro Enzymatic Activity of Recombinant Human SHP2 Protein

### Experimental Procedures

Protein purification: The recombinant DNA for full-length human SHP2 (amino acid 1-597) or the phosphatase domain of human SHP2 (amino acid Ala237-Ile529) was cloned into a pET30 vector (Sigma #69909-3), which was modified to contain a TEV protease site after the N-terminal 6×His tag. The expression vector was transformed into E. coli BL21 (DE3) (NEB #C2527H). The protein was expressed and purified by the following procedure. Bacterial cells from a 50 mL overnight culture were inoculated in 6 L of terrific broth media. Once the E. coli culture reached an OD600 of 0.7, the temperature was lowered to 18° C. and recombinant protein expression was induced by the addition of 0.5 mM IPTG. After overnight incubation, the cells were harvested by centrifugation at 20,000 rpm for 45 min in a Sorvall Lynx 6000 (Thermo Fisher) centrifuge with F9-6×1000 LEX rotor. The cell pellets were stored at −80° C. until purification. The frozen cell pellet was re-suspended in 50 mM HEPES, 500 mM NaCl, 5 mM imidazole, 5% (v/v) glycerol and 0.5 mM TCEP at pH 7.5, containing Roche complete protease inhibitor (Sigma #5056489001), DNase (20 μg/mL, Gol Biotechnology #D-300-5) and lysozyme (0.5 mg/mL, Gold Biotechnology #L-040-25). The cells were lysed on ice by sonication using a Branson digital sonifier at 60% amplitude for 5 min. The debris was pelleted at 20,000 rpm at 4° C. for 1 hour using a F2-12×50 LEX rotor and a Sorvall Lynx 6000 (Thermo Fisher) centrifuge. The supernatant was loaded onto a column containing 2 mL of Ni-NTA-agarose resin (Qiagen #30230). The column was washed with 100 m of 50 mM HEPES, 500 mM NaCl, 5 mM imidazole, 5% (v/v) glycerol, 0.5 mM TCEP at pH 7.5 buffer, followed by a second wash step using 100 mL of 50 mM HEPES, 500 mM NaCl, 30 mM imidazole, 5% (v/v) glycerol, 0.5 mM TCEP at pH 7.5 buffer. The SHP2 protein was eluted using 50 mM HEPES, 500 mM NaCl, 250 mM imidazole, 5% (v/v) glycerol, 0.5 mM TCEP at pH 7.5. The fractions containing SHP2 protein were pooled and TEV protease added at 1 mg TEV for every 10 mg of SHP2 protein. The protein solution was subsequently dialyzed overnight at 4° C. against 50 mM HEPES, 500 mM NaCl, 5 mM imidazole, 5% (v/v) glycerol, 0.5 mM TCEP at pH 7.5 to remove the imidazole and allow the TEV protease to cleave the 6×His-tag, using Snakeskin dialysis tubing (Thermo Fisher #68100). The protein solution was passed through the Ni-NTA column, collecting the flow-through that contained the SHP2 protein with 6×His tag cleaved. This protein solution was further purified in 20 mM Tris, 100 mM NaCl and 2 mM TCEP at pH 7.5 using a 120 mL S75 size exclusion column (G.E. life sciences #17104401). The protein was concentrated to 10 mg/mL using Amicon Ultra centrifugal concentrators (Sigma #UFC901096) and flash frozen. Protein aliquots were stored at −80° C. until required. The final product was 98% pure as determined by SDS-PAGE. The concentration of SHP2 protein was determined from the absorbance at 280 nm wavelength, using the calculated molar extinction coefficient for SHP2 protein of 72770 M⁻¹ cm⁻¹.

SHP2 enzymatic assay: Phosphatase activity of full-length human SHP2 wild-type enzyme or SHP2 phosphatase domain was measured using fluorogenic 6,8-difluoro-4-methylumbelliferyl phosphate (DiFMUP; Molecular Probes #D6567) as the substrate. Stock solution of Compound (10b) was prepared, followed by 1:3 serial dilutions in DMSO (Sigma #D2650). Compounds were further diluted in assay buffer (62.5 mM HEPES, 125 mM NaCl, 1 mM EDTA, 1.25 mM TECP, 0.1% BSA) and aliquots were dispensed into 384-well black plate (Greiner #784900) for a final DMSO concentration of less than 0.5%. Purified full-length SHP2 protein (250 pM) was incubated with or without increasing concentrations of Compound (10b) in assay buffer (10-point dilution, final concentration up to 50,000 nM) containing 1 μM bistyrosylphorphorylated peptide (Sequence: H-LN(pY)IDLDLV(dPEG8)LST(pY)ASINFQK-NH2), for 30 min at room temperature. Reaction was initiated by addition of DiFMUP (50 μM) at room temperature to a final reaction volume of 20 μL in assay buffer. After 1 hour, DiFMUP fluorescence signal was measured (Ex: 340/Em: 460) using an Envision plate reader (Perkin Elmer). The assay with SHP2 phosphatase domain was conducted with 200 pM SHP2 237-529 in a similar manner, except 1 μM bistyrosylphorphorylated peptide was not used in the assay.

Data analysis: Dose-response curves were analyzed using IC₅₀ regression curve fitting (GeneData Screener software). Curves were normalized to a high control without inhibitor and a low control without substrate. The half maximal inhibitory concentration (IC₅₀) was determined, and assay results were uploaded into ChemCart (DeltaSoft) software.

### Results

### Compound (10b) Suppresses the Enzymatic Activity of Purified Human Full-Length SHP2 Protein In Vitro

Full-length SHP2 wild-type enzyme assumes an auto-inhibited conformation. In order to assess the phosphatase activity and subsequent compound mediated inhibition, the enzymatic assay was conducted in the presence of a bistyrosylphorphorylated peptide, which binds to the SH2 domains of SHP2 and induces allosteric activation of the phosphatase activity. Under this assay condition, Compound (10b) potently inhibited SHP2 activity in a dose-dependent manner and displayed an IC₅₀ of 13.2±10.6 nM.

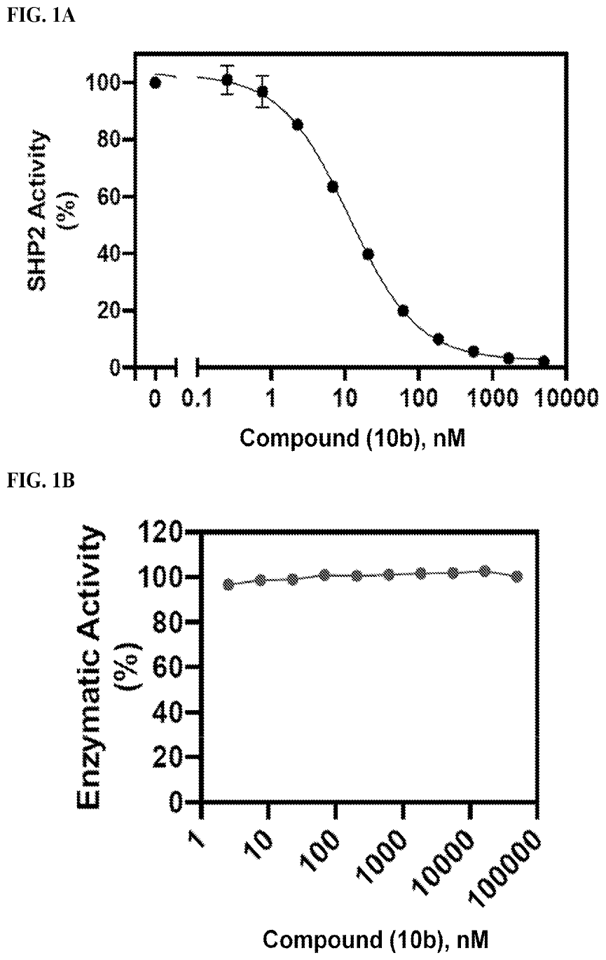

FIG. 1A shows representative dose-response curve of Compound (10b) mediated inhibition of SHP2 activity in the presence of 1 μM bistyrosylphorphorylated peptide in an in vitro enzymatic assay. The dose-response curve represents mean±SEM from two replicates in one representative experiment. Compound (10b) showed an IC₅₀ of 13.2 nM±10.6 nM (mean±SD) calculated from 34 independent test occasions. As shown in FIG. 1A, Compound (10b) potently inhibits the activity of recombinant full-length SHP2 wild-type enzyme, in an in vitro enzymatic assay.

Compound (10b) does not Suppress the In Vitro Enzymatic Activity of SHP2 Phosphatase Domain

FIG. 1B shows dose-response curve of Compound (10b) on the phosphatase activity of the phosphatase domain of human SHP2, in an in vitro enzymatic assay. The dose-response curve represents mean±SEM from six replicates in one experiment. Compound (10b) at up to 50 μM did not suppress the enzymatic activity of SHP2 phosphatase domain. As shown in FIG. 1B, Compound (10b) does not inhibit the activity of a truncated form of SHP2 that contains the phosphatase domain, in an in vitro enzymatic assay.

### Conclusions

Compound (10b) is a potent inhibitor of purified full-length human SHP2 wild-type enzyme and displays an IC₅₀ of 13.2±10.6 nM. It does not suppress the enzymatic activity of truncated SHP2 that contains the phosphatase domain, suggesting regions outside the phosphatase domain are required for Compound (10b) binding.

### Example 2: Selectivity of Compound (10b)

Activity of Compound (10b) (1 μM) was assessed against a panel of 419 human kinases (including a set of disease-relevant mutant variants). Assays were conducted using the KINOMEscan™ platform from DiscoverX which measures compound interaction with kinases using active site-directed competition binding assays. Displacement of ligand binding to the kinase by <35% of the control value is considered significant. Of the 419 kinases tested, only 4 kinases reached this level of displacement: Protein kinase C eta (PRKCH), 34% control; Large tumor suppressor kinase 1 (LATS1), 32% control; General control nonderepressible 2 kinase (GCN2), 21% control; and Hematopoietic Progenitor Kinase 1 (HPK1), 4.4% control.

A dose-response curve was performed for HPK1 kinase and the K_{d} of Compound (10b) for binding to this kinase was determined to be 9.8 μM. HPK1 is a hematopoietic cell-restricted serine/threonine kinase and is not expressed in any major organs. HPK1 has been implicated in activation of c-Jun N-terminal kinases (JNK) and genetic disruption of HPK1 is reported to enhance the antitumor immune response of T cells and dendritic cells (Hu et al 1996; Sawasdikosol et al 2012).

The activity of Compound (10b) was also assessed against a panel of 14 full-length human phosphatases using spectrofluorimetric biochemical assays. These assays were conducted using the Eurofins Panlabs platform. No significant inhibition of any of these phosphatases was observed at 10 μM Compound (10b). At this concentration, Compound (10b) did not demonstrate any inhibition of human SHP-1.

In addition to the kinase and phosphatase screens, Compound (10b) (10 μM) was tested against a panel of 73 receptors and ion channels and 12 additional enzymes using the Eurofins Cerep platform. In this screen, inhibition or stimulation of >50% is considered to be a significant effect. No effects of this magnitude were observed for any of the targets studied.

These results indicate that Compound (10b) is a selective inhibitor of SHP2 with no significant off-target activities.

The potential for Compound (10b) to inhibit hERG potassium channel current was evaluated in vitro in Chinese hamster ovary (CHO) cells using manual whole cell patch clamp technology. CHO cells stably transfected with hERG cDNA and expressing active hERG potassium channels were plated on glass cover slips and placed in culture dishes for electrophysiological recording of hERG current without and with Compound (10b) at concentrations of 0.3, 1, 3, 10, 30, and 100 μM (n=3/concentration).

HERG channel currents were recorded from single cells using standard whole cell recording techniques. The cells were voltage clamped at a holding potential of −80 mV. The hERG current was activated by depolarizing at +20 mV for 5 sec, after which the current was taken back to −50 mV for 5 sec to remove the inactivation and observe the deactivating tail current. The K+ tail current through HERG channels observed during this step was allowed to stabilize under continuous bath perfusion. Cells were then superfused with Compound (10b) until steady state block was achieved. Steady state was considered reached when three consecutive super-imposable current records were collected. Cisapride was used in the experiments as a positive control for hERG inhibition to ensure normal response and good quality of the hERG cells.

Compound (10b) had no effect on hERG channel activity at concentrations from 0.3 to 30 μM. At 100 μM, Compound (10b) inhibited hERG activity by 25.7% relative to the vehicle control (DMSO), indicating an IC₅₀ of >100 μM. In contrast, cisapride inhibited hERG current by >50% at 0.1 μM.

### Example 3: Single-Dose Pharmacokinetics of Compound (10b)

The pharmacokinetic (PK) parameters of Compound (10b) (freebase) were determined following single intravenous (IV) and oral (PO) administration in female CD-1 mouse, female C57BL/6 mouse (PO only), male Sprague Dawley rat, male Beagle dog, and male Cynomolgus monkey. Additional evaluation of the PK properties of Compound (10b) was obtained following single dose PO administration in female CD-1 mouse, male Sprague Dawley rat and male Beagle dog over a dose range spanning 10 to 300 mg/kg (mouse and rat) and 1 to 100 mg/kg (dog). The intravenous dose solutions were prepared in 20% DMSO-60% PEG400-20% water and the oral dose solutions as suspensions in 0.5% methylcellulose in water, unless otherwise noted. Plasma concentration profiles were generated from serial sampling in individual animals up to 24 hours post dose, except for the studies in mice where composite sampling methods were used (i.e. 3 sampling times per mouse; 9 mice per group). Compound (10b) exhibited moderate plasma clearance in CD-1 mouse (1.54 L/h/kg), and low plasma clearance in Sprague Dawley rat, Beagle dog, and Cynomolgus monkey (0.61, 0.51 and 0.36 L/h/kg, respectively). The apparent volumes of distribution at steady-state were moderate across species, ranging from 2.74 L/kg (monkey) to 6.05 L/kg (rat). Terminal elimination half-life (T_{1/2}) values were 2.21, 9.18, 8.23, and 6.44 hours for CD-1 mouse, Sprague Dawley rat, Beagle dog and Cynomolgus monkey, respectively. Following oral administration at doses of 1-100 mg/kg, Compound (10b) was absorbed relatively rapidly across species, with mean Tₘₐₓ values ranging from 0.25 to 3.67 hours postdose. At 300 mg/kg, the highest dose administered in CD-1 mouse and Sprague Dawley rat, the mean Tₘₐₓ values remained 0.25 hours in mouse, while in increased up to 8 hours in rat. Apparent oral bioavailabilities, reported for oral doses that were 3- to 30-fold higher than the corresponding intravenous doses, were moderate to high (51.2-115%) across species. Exposures increased with increasing dose level across species, in a dose-proportional manner in mouse and dog, and in a greater than dose-proportional manner in rat (80× increase in AUC with a 30× increase in dosage).

TABLE 1
Pharmacokinetic Parameters for Compound (10b)
(free base) in Mouse, Rat, Dog and Monkey
Following Intravenous and Oral Administration
Intravenous

| | Dose | Clₚ | Vdₛₛ | T_{1/2} | AUC_{0→24} |
|---|---|---|---|---|---|
| Species (strain, sex) | (mg/kg) | (L/h/kg) | (L/kg) | (hr) | (μM*hr) |
| Mouse (CD-1, female) | 1 | 1.54 | 3.84 | 2.21 | 1.39 |
| Rat (SD^{a}, male) | 0.3 | 0.61 | 6.05 | 9.18 | 3.27 |
| Dog (Beagle, male) | 1 | 0.51 | 5.46 | 8.23 | 3.91 |
| Monkey (cynomolgus, | 0.3 | 0.36 | 2.74 | 6.44 | 1.82 |
| male) | | | | | |

Oral

| | Dose | Tₘₐₓ ^{b} | F | Cₘₐₓ | AUC_{0→24} |
|---|---|---|---|---|---|
| Species (strain, sex) | (mg/kg) | (h) | (%) | (μM) | (μM*hr) |
| Mouse (CD-1, female) | 10 | 0.25 | 84.1 | 4.17 | 12.5 |
| Mouse (C57BL/6, female) | 10 | 1.00 | — | 5.40 | 39.0 |
| Rat (SD^{a}, male) | 10 | 0.92 | 51.2 | 3.05 | 19.1 |
| Dog (Beagle, male) | 10 | 0.83 | 115 | 6.37 | 48.2 |
| Monkey (cynomolgus, | 1 | 2.67 | 52.3 | 0.43 | 4.52 |
| male) | | | | | |

^{a}Sprague Dawley; and
^{b} Mean Tₘₐₓ values

### Example 4: In Vivo Pharmacodynamics of Compound (10b) in KYSE-520 Tumors

Treatment with Compound (10b) Suppresses DUSP6 mRNA Levels in the KYSE-520 Xenograft Tumors a Dose-Dependent Manner

KYSE-520 (EGFRamp) cells were subcutaneously implanted in NSG mice and allowed to grow to an average tumor volume of 340 mm³ as monitored by caliper measurements. At this point, animals were randomized and treated with vehicle, Compound (10b) 25 mg/kg or Compound (10b) 100 mg/kg PO. Plasma and tumor samples were harvested 4, 16 and 24 hours after a single dose. Compound (10b) plasma concentration and tumor pERK levels were determined. Data represent mean±SEM. N=4 mice per group.

When female NSG mice bearing subcutaneous KYSE-520 tumors were treated with a single oral dose of Compound (10b) at 25 mg/kg or 100 mg/kg, dose-dependent and time-dependent suppression of DUSP6 mRNA levels in the tumors was observed. At 4-hour after a single dose, treatment with Compound (10b) at both 25 mg/kg and 100 mg/kg potently suppressed DUSP6 mRNA levels (>90% suppression). At 16-hour after a single dose, treatment with Compound (10b) at 25 mg/kg moderately suppressed DUSP6 mRNA levels (<50% suppression) and at 100 mg/kg significantly suppressed DUSP6 mRNA levels (>50% suppression). At 24-hour after a single dose, neither treatment significantly suppressed DUSP6 mRNA levels.

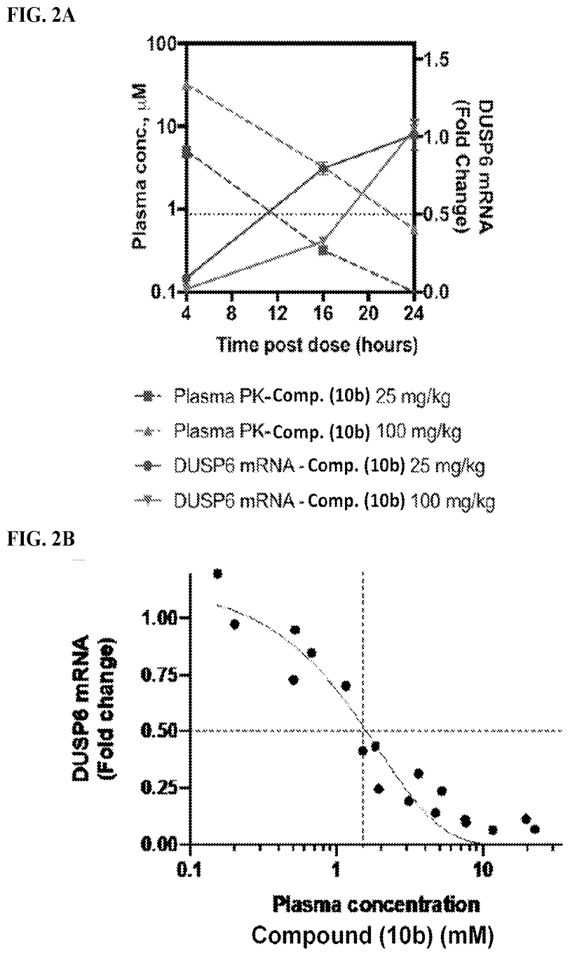

Dose-dependent suppression of DUSP6 mRNA by Compound (10b) treatment was also observed. Importantly, an inverse correlation was observed between DUSP6 mRNA level (solid lines in FIG. 2A) and plasma concentration of Compound (10b) (dashed lines in FIG. 2A). For both doses, as the plasma concentration decreased from 4-hour to 24-hour after dosing, tumor DUSP6 mRNA levels increased over time. Together, the data suggest that treatment with Compound (10b) suppressed DUSP6 mRNA levels in the KYSE-520 tumors in a dose-dependent manner, and tumor DUSP6 mRNA levels were inversely correlated with Compound (10b) concentrations in the plasma.

FIGS. 2A-2B show PK/PD relationship and IC₅₀ determination in KYSE-520 treated with Compound (10b). FIG. 2A: PK/PD relationship; and FIG. 2B: IC₅₀ determination.

Compound (10b) Suppresses mRNA Levels of MPAS-Plus Genes in the KYSE-520 Xenograft Tumors in a Dose-Dependent Manner

In addition to DUSP6 mRNA levels, other MAPK pathway genes were examined through sequencing analysis. The MPAS (MAPK pathway activity score) signature is a signature of 10 genes that reflects MAPK pathway activity. This gene signature has been used in the clinic to evaluate the pharmacodynamic effect of the ERK inhibitor GDC-0994 in tumors. Based on the MPAS signature, a 13-gene signature (“MPAS-plus”) was developed, which includes the 10 MPAS genes and three additional MAPK-targeted genes (ETV1, EGR1 and FOSL1) that are modulated by SHP2 inhibitors across multiple cell line models (data not shown).

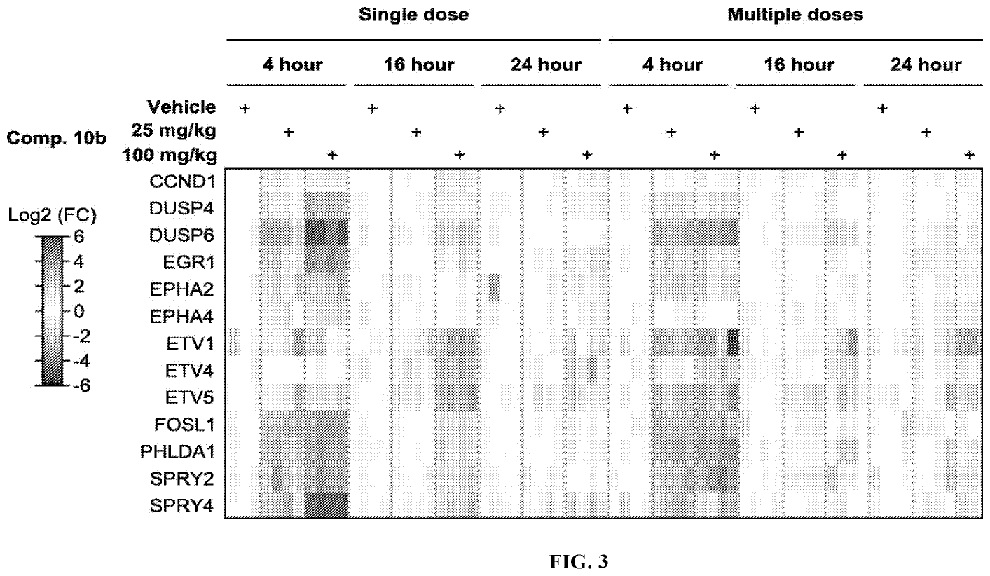

When female NSG mice bearing subcutaneous KYSE-520 tumors were treated with a single oral dose or five doses of Compound (10b) at 25 mg/kg or 100 mg/kg, dose- and time-dependent suppression of the MPAS-plus signature in the tumor was observed. Overall, the mRNA levels of the majority of the MPAS-plus genes was most potently suppressed at 4-hour after both a single dose and the fifth dose, and the suppression decreased from 4-hour to 24-hour (FIG. 3), as the plasma concentration decreased. Dose-dependent suppression was observed with many genes in addition to DUSP6 after treatment with both a single dose and five doses of Compound (10b) (e.g., ETV1, ETV4 and PHDLA1, at 16-hour after treatment). Among these 13 genes, some genes were more potently suppressed by Compound (10b) (e.g., DUSP6 and SPRY4), and some genes were suppressed by Compound (10b) to a lesser extent (e.g., CCND1 and EPHA2) in the KYSE-520 tumor. Biological adaptation appeared to be more obvious with some transcripts such as FOSL1 than DUSP6 (compare 16-hour after single dose treatment vs 16-hour after the fifth dose). DUSP6 mRNA data from the sequencing analysis (FIG. 3) was identical to that from qRT-PCR. Evaluating the MPAS-plus signature that includes DUSP6 may be of advantage in capturing the overall activity of the MAPK pathway signaling in the heterogeneous patient tumors in the clinical trial.

### Example 5: In Vivo Pharmacodynamics of Compound (10b) in HCC827 (EGFR^{ex19del} & EGFR^{amp}) CDX Model

Male NOD/SCID mice (n=3 per group) were administered a single dose of Compound (10b) at 1, 3, 10, 30, and 100 mg/kg by oral gavage. Plasma was collected at 0.25, 1, 2, 4, 8, and 24 hours after each dose, and brains were collected at 24 hours after each dose. Compound (10b) concentrations were determined with LC-MS/MS.

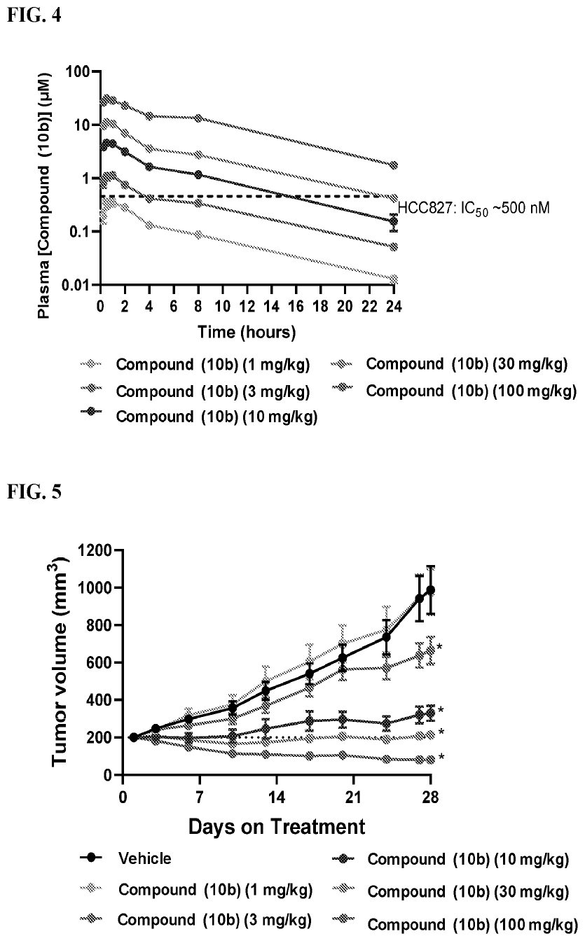

FIG. 4 shows a dose-relation of Compound (10b) in male NOD/SCID mice after single oral doses and the duration over in vitro NSCLC HCC827 PERK IC₅₀. The mean Cₘₐₓ increased proportionally with dose (Cₘₐₓ for doses 1, 3, 10, 30, and 100 mg/kg were 160, 507, 2033, 4873, and 13900 ng/mL, respectively). AUC_{inf} increased proportionally with dose (AUC_{inf} for doses 1, 3, 10, 30, and 100 mg/kg were 1031, 3530, 13047, 30596, and 123116 hr*ng/mL, respectively). Total brain/plasma levels at 24 hours for doses 1, 3, 10, 30, and 100 mg/kg were 0.35, 0.31, 0.30, 0.31, and 0.37, respectively. Compound (10b) AUC and Cₘₐₓ increased proportionally with dose indicating Compound (10b) has no concentration dependent effect on absorption, binding, or clearance in the dose range tested. Compound (10b) mean total brain/plasma level at 24 hours was 0.33, also indicating Compound (10b) has no concentration dependent effect on brain distribution in the dose range tested. As shown in FIG. 4, 10 mg/kg of Compound (10b) was sufficient to maintain target coverage over the in vitro pERK IC₅₀ of the NSCLC HCC827 cell line for 16 hours.

An in vivo efficacy study was conducted to evaluate the monotherapy anti-tumor activity of Compound (10b) in female BALB/c nude mice bearing the non-small cell lung cancer (NSCLC) HCC827 cell line-derived xenograft (CDX) model, which harbors an EGFR exon 19 deletion (EGFR^{ex19del}) and EGFR amplification (EGFRamp). Mice (n=10 per group) were dosed daily orally with the indicated levels of Compound (10b) from day 1 to day 28, and a dose dependent reduction in tumor volume was observed. FIG. 5 shows monotherapy anti-tumor activity of Compound (10b). All groups dosed QD, PO Two-way mixed-effects ANOVA: *p<0.0001 vs vehicle.

As shown in FIG. 5, the 3, 10, and 30 mg/kg Compound (10b) groups had statistically significant monotherapy anti-tumor activity compared to the vehicle group with 41%, 84%, 98% tumor growth inhibition (TGI), respectively. The 100 mg/kg Compound (10b) group also had statistically significant monotherapy anti-tumor activity compared to the vehicle group, with 60% mean tumor regression. The ED₉₀ for Compound (10b) in this model was 10 mg/kg, confirming that ˜16 hours over the pERK IC₅₀ drives efficacy in this xenograft model. There was no impact of treatment on body weight and all treatments were well tolerated.

### Example 6: In Vivo Pharmacodynamics of Compound (10b) in Various Cell Lines

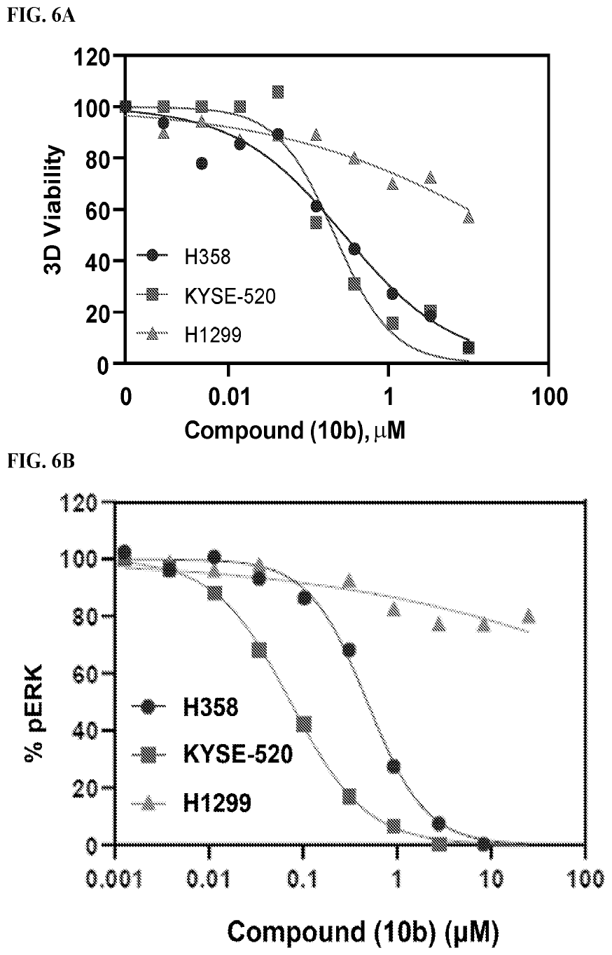

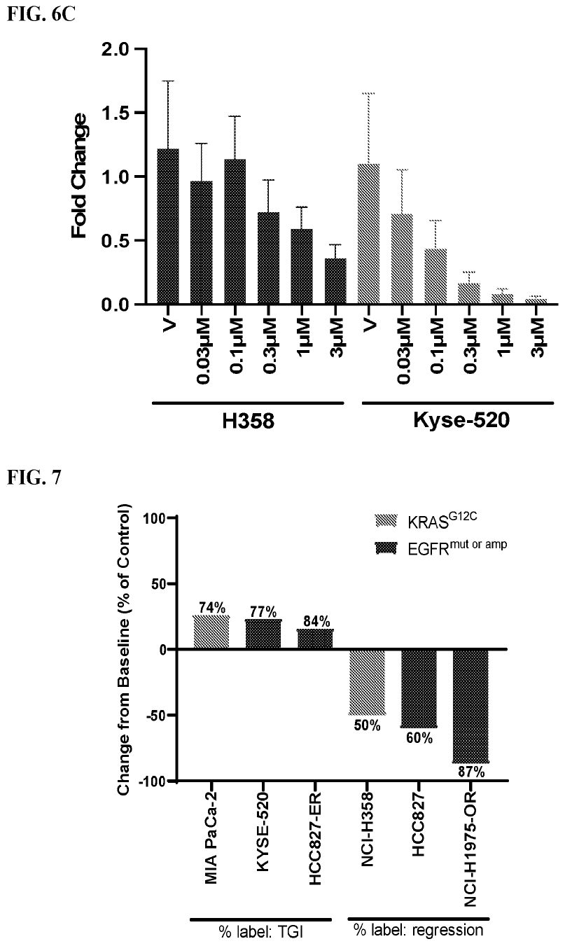

The potency of Compound (10b) in a panel of human tumor cell lines with active MAPK signaling was assessed. Cell viability in a 3D culture system was assessed by measuring CellTiter-Glo (CTG) luminescence following 8 days of treatment with nine concentrations of Compound (10b). MAPK pathway signaling was assessed by measuring pERK protein levels and DUSP6 mRNA levels by homogeneous time resolved fluorescence (HTRF) and quantitative polymerase chain reaction (qPCR), respectively. Potent, dose responsive, cell viability effects were observed in NCI-H358, a KRAS^{G12C} mutant cell line, and KYSE-520, an EGFR amplified (EGFRamp) cell line, following Compound (10b) treatment (FIG. 6A). These viability effects correlated with pERK inhibition (Table 2, FIG. 6B) and DUSP6 inhibition (FIG. 6C). Potent pERK and viability effects were also observed in additional EGFR mutant cell lines HCC827 and NCI-H1975, but minimal effects were observed in NCI-H1299, a cell line containing NRAS^{Q61K}, a mutation that has been shown to confer resistance to SHP2 inhibition.

TABLE 2
Inhibition of pERK by Compound (10b)

| | | DUSP6 | pERK | 3D CTG |
|---|---|---|---|---|
| Cell Line | Mutation | (IC₅₀, nM) | (IC₅₀, nM) | (IC₅₀, nM) |
| KYSE-520 | EGFR^{amp} | 60 | 73 | 210 |
| NCI-H1975 | EGFR^{L858R/T790M} | — | 400 | 180 |
| HCC827 | EGFR^{Ex19del} | — | 460 | 1200 |
| NCI-H358 | KRAS^{G12C} | 820 | 480 | 280 |
| NCI-H1299 | NRAS^{Q61K} | — | >25,000 | >25,000 |

In vivo efficacy studies were conducted to evaluate the monotherapy anti-tumor activity of Compound (10b) across a panel of cell line-derived xenograft (CDX) models. Immunocompromised mice (n=5-10 per group) bearing the indicated CDX tumors were dosed daily orally with 100 mg/kg Compound (10b) for 21 to 33 days and statistically significant Compound (10b) significant monotherapy anti-tumor activity compared to the vehicle group was observed in all CDX models (FIG. 7). In the KRAS^{G12C} mutant CDX models MIA PaCa-2 and NCI-H358, 74% tumor growth inhibition (TGI) and 50% mean tumor regression, were observed, respectively. In the models with EGFR alterations, 77% TGI was observed in the EGFR amplified (EGFR^{amp}) KYSE-520 model and 60% mean tumor regression was observed in the EGFR exon 19 deletion (EGFR^{ex19del}) and EGFR amplified (EGFR^{amp}) HCC827 model. Significant anti-tumor efficacy was also observed in two CDX models with acquired resistance to EGFR inhibitors. 84% TGI was observed in the HCC827-ER CDX model, which has acquired resistance to an EGFR inhibitor such as erlotinib and osimertinib through MET amplification (MET^{amp}), and 87% mean tumor regression was observed in the NCI-H1975-OR CDX model which bears EGFR^{L858R} and EGFR^{T790M} mutations and also has an engineered EGFR^{C797S} mutation that confers resistance to osimertinib. There was no impact of treatment on body weight and all treatments were well tolerated.

### Example 7: A Phase 1/1B First-in-Human Study of the SHP2 Inhibitor Compound (10b) in Patients with Advanced Solid Tumors

Study Title: A Phase 1/1B First-in-Human Study of the SHP2 Inhibitor Compound (10b) in
Patients with Advanced Solid Tumors
Study Phase: Phase 1/1b
OBJECTIVES:
Primary Objective:
The primary objective of this study is to evaluate the safety, tolerability and maximum tolerated
dose (MTD) and establish the recommended phase 2 dose (RP2D) of Compound (10b), a SHP2
inhibitor, in patients with specific advanced or metastatic solid tumors.
Secondary Objectives:
The secondary objectives of the study are:
To assess the preliminary anti-tumor activity of Compound (10b), as defined by the
objective response rate (ORR, complete response + partial response rate), the duration of
response (DOR), and progression free survival (PFS)
To assess the pharmacokinetic (PK) and pharmacodynamic profile of Compound (10b)
To evaluate the effect of a standardized high-fat/high-calorie meal on the
pharmacokinetics of Compound (10b)
Exploratory Objectives:
The exploratory objectives of the study are:
To assess biomarkers that correlate with response
To characterize Compound (10b) excretion in urine
Details of the study, including enrollment, fasting requirements, performance of a Food
Effect/PK study, and scheduled assessments, are subject to change
STUDY CENTERS:
Dose Escalation: Approximately 5
Dose Expansion: Approximately 20
Food Effect (FE) and PK Sub-study (selected centers): Approximately 10
NUMBER OF PATIENTS PLANNED:
Dose Escalation: Approximately 25 patients are anticipated to be enrolled.
Dose Expansion: Up to 80 patients are anticipated to be enrolled in 4 expansion cohorts with up
to 20 patients each.
Food Effect and PK Sub-study (optional): Up to 24 patients may be enrolled in up to 3 FE/PK
cohorts with up to 8 patients each.
Total Enrollment: Up to approximately 130 patients.
PATIENT ELIGIBILITY
NOTE: All inclusion and exclusion criteria apply to the Dose Escalation part of the study, the
Dose Expansion part of the study, and the optional FE/PK sub-study, unless otherwise stated.
Inclusion Criteria

| | |
|---|---|
| 1. | Male and non-pregnant females ≥18 years old. |
| 2. | Patients must have a diagnosis of advanced (primary or recurrent) or metastatic solid tumor |
| | with MAPK-pathway alterations (excluding BRAF V600X), as assessed by molecular |
| | diagnostics using an appropriate clinically validated and/or FDA approved test, and have no |
| | available standard of care or curative therapies (MAPK-pathway alterations include, for |
| | example KRAS G12C-mutant; epidermal growth factor receptor (EGFR)-mutant; see |
| | Appendix 1). |
| 3. | Dose Expansion Only: Patients must have one of the following genomically- and |
| | histologically-defined tumor types: |
| a. | Cohort A: Advanced or metastatic KRAS^{G12C} mutant non-small cell lung cancer |
| | (NSCLC) with no available standard of care or curative therapies |
| b. | Cohort B: Advanced or metastatic KRAS^{G12C} mutant solid tumor other than NSCLC with |
| | no available standard of care or curative therapies |
| c. | Cohort C: Advanced or metastatic NF1 loss-of-function (LOF) solid tumor with no |
| | available standard of care or curative therapies |
| d. | Cohort D: Advanced or metastatic EGFR-mutant NSCLC, that progressed on standard of |
| | care EGFR TKI therapies, with no available standard of care or curative therapies |
| 4. | Patients must have measurable disease by RECIST v1.1. |
| 5. | Patients must have an Eastern Cooperative Oncology Group performance status of 0-2 (see |
| | Appendix 2). |
| 6. | Patients must have adequate organ function, as defined below: |

Hematological

| | |
|---|---|
| a. | absolute neutrophil count ≥1,500/μL |
| b. | platelets ≥100,000/μL |
| c. | hemoglobin ≥9 g/dL without transfusion for >2 weeks or erythropoiesis-stimulating |
| | agents (eg, Epo, Procrit) for >6 weeks |

Renal

| | |
|---|---|
| d. | Estimated glomerular filtration rate ≥60 mL/min/1.73 m² (calculated by the Chronic |
| | Kidney Disease Epidemiology Collaboration equation) |

Hepatic

| | |
|---|---|
| e. | Serum total bilirubin <2.0 × institutional upper limit of normal (ULN) or <3.0 × |
| | institutional ULN if the individual has a diagnosis of Gilbert syndrome or hemolytic |
| | anemia as confirmed by the investigator |
| f. | Aspartate aminotransferase/serum glutamic-oxaloacetic transaminase (AST/SGOT) |
| | and/or alanine aminotransferase/serum glutamic-pyruvic transaminase (ALT/ |
| | SGPT) ≤2.5 × ULN or ≤5 × ULN in the presence of liver metastases |

Coagulation

| | |
|---|---|
| g. | International normalized ratio (INR) or prothrombin time (PT) ≤1.5 × ULN unless the |
| | patient is receiving anticoagulant therapy and as long as PT or activated partial |
| | thromboplastin time (aPTT) is within the therapeutic range of intended use of |
| | anticoagulants. |
| h. | Activated partial thromboplastin time ≤1.5 × ULN unless the patient is receiving |
| | anticoagulant therapy and as long as PT or aPTT is within the therapeutic range of |
| | intended use of anticoagulants. |
| 7. | Women of childbearing potential (WOCBP) MUST have a negative serum human chorionic |
| | gonadotropin test unless permanently sterile. Permanent sterilization methods include prior |
| | tubal ligation (≥1 year prior to screening), bilateral salpingectomy, bilateral oophorectomy, |
| | total hysterectomy or menopause (defined as 12 consecutive months of amenorrhea and |
| | confirmed by follow up hormone level assessment). |
| 8. | Female patients of childbearing potential not permanently sterilized must use 2 methods of |
| | contraception for the duration of the study and for at least 90 days after the last dose of |
| | investigational product (IP). Male patients with female partners must use contraception for |
| | the duration of the study and for at least 105 days after the last dose of IP. Female patients or |
| | female partners of male patients should not become pregnant or breastfeed while on this |
| | study. |
| 9. | Patients must have the ability to understand and the willingness to sign a written informed |
| | consent document (prior to the initiation of the study and any study procedures). |
| 10. | Patients must be willing and able to comply with the scheduled visits, treatment plan, |
| | laboratory tests, and other specified study procedures in the opinion of the investigator |

Exclusion Criteria

| | |
|---|---|
| 1. | Patients with active Hepatitis B infection (defined by the presence of hepatitis B surface |
| | antigen [HBsAg)] or presence of hepatitis B virus DNA), Hepatitis C infection (defined by |
| | the presence of hepatitis C virus [HCV] antibody and positive HCV RNA), or human |
| | immunodeficiency virus (HIV) infection with measurable viral load. |
| 2. | Patients with active infection requiring intravenous antibiotics or other uncontrolled |
| | intercurrent illness requiring hospitalization. Minor infections (eg, periodontal infection or |
| | urinary tract infection) which may be treated with short term oral antibiotics are allowed. |
| 3. | Patients with any of the following cardiac-related issues or findings: |
| a. | History of significant cardiovascular disease, such as cerebrovascular accident, |
| | myocardial infarction or unstable angina, within the last 6 months before starting therapy. |
| b. | Clinically significant cardiac disease, including New York Heart Association Class II or |
| | higher heart failure. |
| c. | History of left ventricular ejection fraction <50% within the previous 12 months. |
| d. | Mean resting corrected QT interval >470 msec obtained from three electrocardiograms |
| | (ECGs), using the provided ECG machine. |
| e. | Any clinically significant abnormalities in rhythm, conduction or morphology of resting |
| | ECG (eg, third degree heart block, Mobitz Type II heart block, ventricular arrhythmias, |
| | uncontrolled atrial fibrillation) |
| 4. | Patients who have participated in an interventional clinical study within the last 4 weeks OR, |
| | if applicable, are within 5 times the half-life of the IP, whichever is lesser, prior to the C1D1 |
| | visit (or C0D1 in the optional FE/PK sub-study). |
| 5. | Have received radiotherapy or proton therapy with a limited field of radiation for palliation |
| | within 1 week of the start of study treatment, or radiation to more than 30% of the bone |
| | marrow or with a wide field of radiation, both within 4 weeks of the start of study treatment. |
| 6. | Patients who have a diagnosis of another invasive malignancy within the previous 3 years |
| | other than curatively treated non-melanomatous skin cancer, superficial urothelial carcinoma, |
| | in situ cervical cancer, or any other curatively treated malignancy that is not expected to |
| | require treatment for recurrence during the course of study. |
| 7. | Patients with tumors harboring known activating mutations in BRAF V600X, PTPN11 |
| | (SHP2), MEK, or RAS Q61. |
| 8. | Patients with known primary central nervous system (CNS) tumors. |
| 9. | Patients with known active CNS metastases and/or carcinomatous meningitis. Patients with |
| | previously treated brain metastases may participate provided they are stable (without |
| | evidence of progression by imaging for at least 4 weeks prior to the first dose of IP and any |
| | neurologic symptoms have returned to baseline), have no evidence of new or enlarging brain |
| | metastases, and are not using steroids and/or anti-seizure medications for at least 7 days prior |
| | to the start of IP. This exception does not include carcinomatous meningitis which is |
| | excluded regardless of clinical stability. |
| 10. | Patients who have undergone major surgery within 4 weeks prior to study enrollment. |
| | Note: This does not include patients who have had procedures such as peripherally inserted |
| | central catheter line placement, thoracentesis, paracentesis, biopsies, or abscess drainage. |
| 11. | Patients who have previously received a SHP2 inhibitor (eg, TNO-155, RMC-4630, RLY- |
| | 1971). |
| | Note: previous treatment with a KRAS^{G12C} inhibitor is not an exclusion criterion. |
| 12. | Patients who have received strong or moderate inducers or inhibitors of cytochrome P450 |
| | (CYP) 3A4 or P-glycoprotein (P-gp) inducers or inhibitors (including herbal supplements) |
| | within 14 days or 5 half-lives (whichever is longer) of C1D1 (or C0D1 in the optional FE/PK |
| | sub-study) (see list provided in Appendix 3). |
| 13. | Patients who take drugs that are known substrates of P-gp, breast cancer resistance protein |
| | (BCRP), multidrug and toxin extrusion protein (MATE) 1 or MATE 2-K, unless stopped 7 |
| | days prior to C1D1 (or C0D1 in the optional FE/PK sub-study) and for the study duration |
| | (see lists provided in Appendix 3 and Appendix 4). |
| 14. | Patients with any condition or therapy, which, in the opinion of the investigator, might pose a |
| | risk to the patient, impair participation or cooperation, or might confound the ability to |
| | interpret study data including if the underlying disease is expected to progress such that |
| | patient is unlikely to complete at least 3 cycles of 28 days of treatment per cycle. |
| 15. | Patients with a life expectancy of ≤12 weeks after the start of IP according to the |
| | investigator's judgement. |
| 16. | Patients with inability to swallow oral medications (capsules, tablets) without chewing, |
| | breaking, crushing, opening or otherwise altering the product dosage form. |
| 17. | Patients with gastrointestinal illness that, in the opinion of the investigator, would preclude |
| | the absorption of Compound (10b), which is an oral agent (eg, post gastrectomy, short bowel |
| | syndrome, uncontrolled Crohn's disease, celiac disease with villous atrophy or chronic |
| | gastritis). |
| 18. | Patients who are on dialysis. |
| 19. | Female patients who are pregnant, planning to become pregnant or who are breastfeeding. |
| 20. | Patients with known intolerance/hypersensitivity to Compound (10b) or its excipients. |
| 21. | Food Effect (FE) Cohort of the Sub-study Only: Patients who have a diet incompatible |
| | with the on-study diet, in the opinion of the investigator. |
| 22. | Food Effect (FE) Cohort of the Sub-study Only: Patients who are lactose intolerant. |

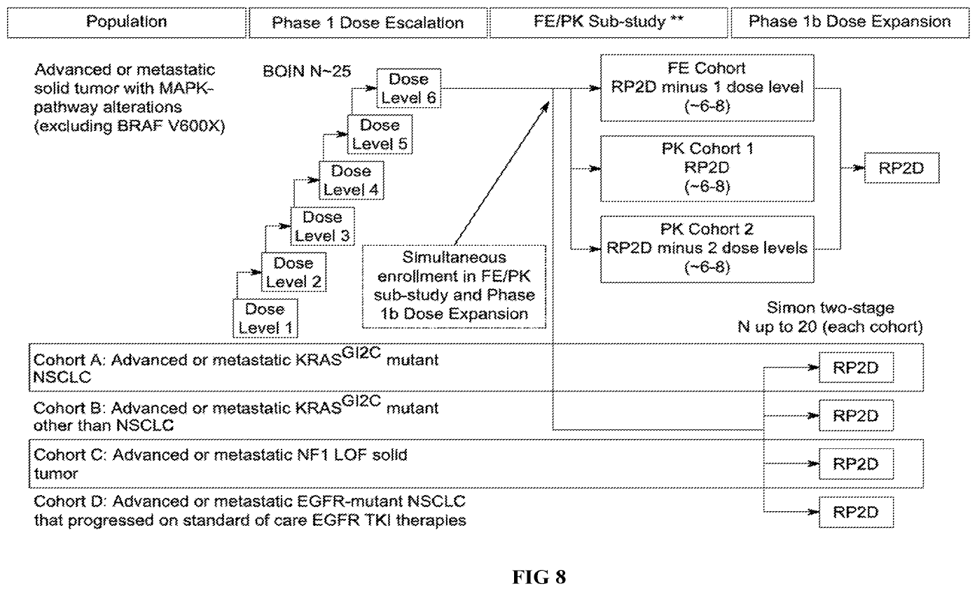

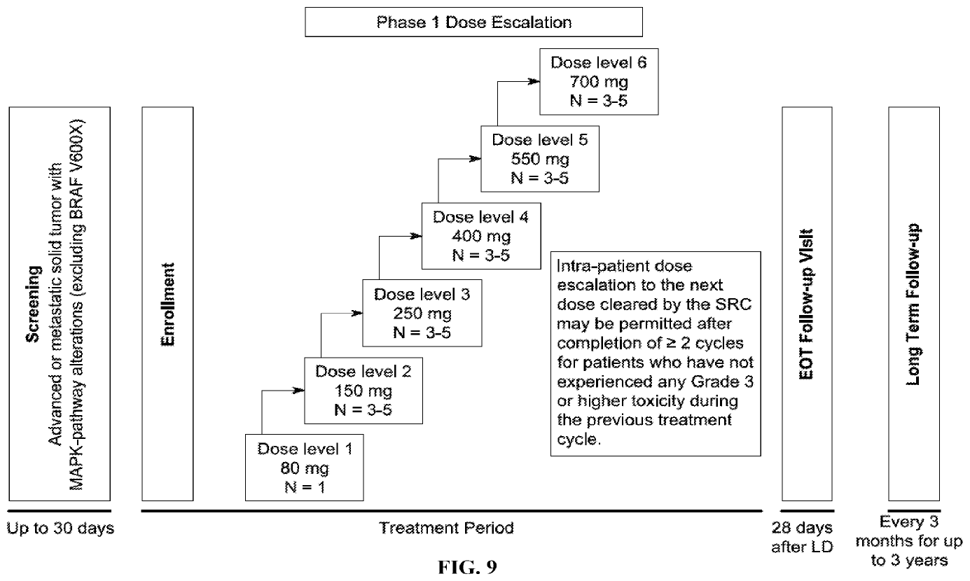

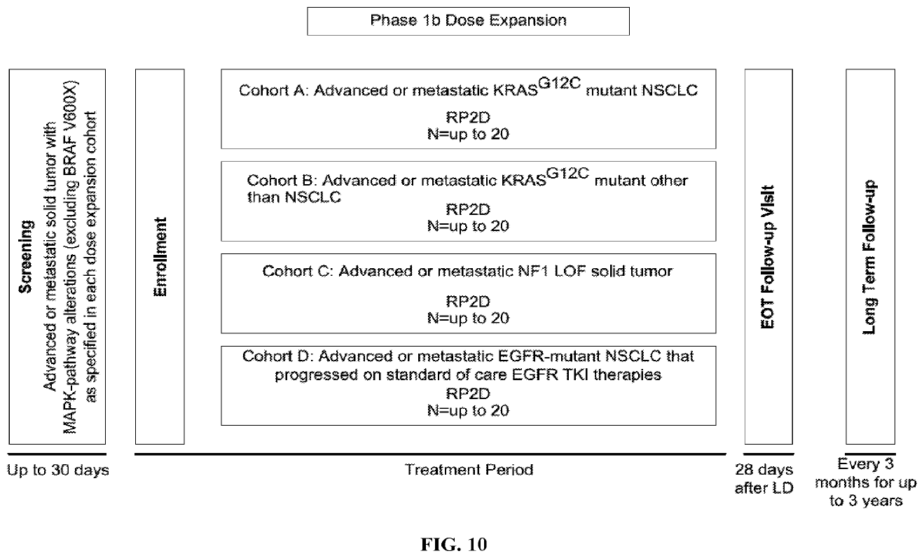

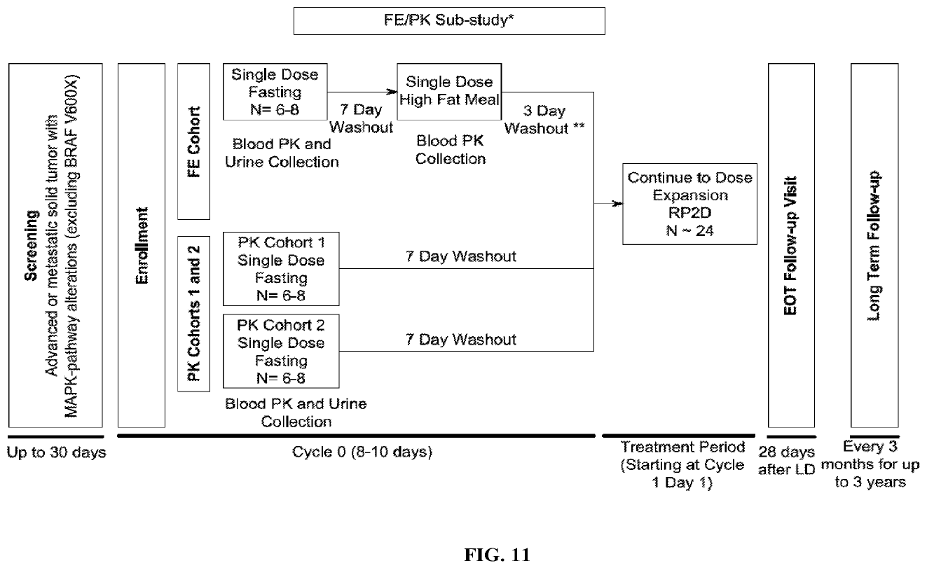

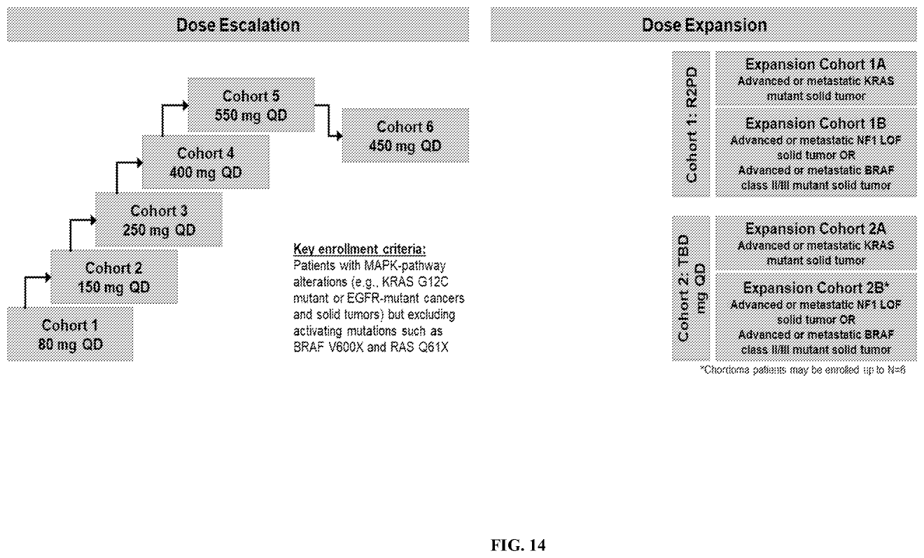

STUDY DESIGN
Overview: This study is an open-label, sequential-cohort, non-randomized, first-in-human,
Phase 1/1b study. The study has 3 parts: Phase 1 Dose Escalation, Phase 1b Dose Expansion, and
an optional FE/PK sub-study, the latter of which will be conducted at selected centers only.
The overall study design is depicted in FIG. 8. Enrollment into the Dose Escalation part of the
study will occur first, followed by concurrent and independent enrollment into the Dose
Expansion part and the optional FE/PK sub-study.
Patients in Dose Escalation and the optional FE/PK sub-study will have advanced or metastatic
solid tumors with MAPK-pathway alterations (excluding BRAF V600X). Patients in Dose
Expansion will be assigned to different study cohorts (Cohorts A, B, C or D), as follows:
Cohort A: Patients with advanced or metastatic KRAS^{G12C} mutant non-small cell lung
cancer (NSCLC).
Cohort B: Patients with advanced or metastatic KRAS^{G12C} mutant solid tumor other than
NSCLC.
Cohort C: Patients with advanced or metastatic NF1 LOF solid tumor.
Cohort D: Patients with advanced or metastatic EGFR-mutant NSCLC, that progressed
on standard of care EGFR TKI therapies.
FIGS. 9-11 show sample designs for the Dose Escalation [FIG. 9], Dose Expansion [FIG. 10],
and optional FE/PK sub-study [FIG. 11]. FIG. 14 shows a sample design for a study including
Dose Escalation and Dose Expansion periods. Each of these sections may consist of a 30-day
screening period; a treatment period consisting of sequential consecutive treatment cycles; a
post-treatment follow-up period of 28 days following the patient's last dose of Compound (10b);
and a long-term follow up period of up to 3 years. It is important to note that after screening,
patients in the FE/PK sub-study will be in the sub-study for up to 10 days (Cycle 0), depending
on the cohort, prior to C1D1.
Dosing will continue for 2 years unless the patient is discontinued from IP or is withdrawn from
the study. The End of Treatment (EOT) Follow-up Visit will be conducted 28 days following the
patient's last dose, unless one of the following occur: death, loss to follow-up, or withdrawal of
consent. Long-term follow-up (with telephone contact every 3 months via telephone) will
continue for up to 3 years unless the patient is withdrawn from study.
The main schedule of assessments for the 3 parts of the study (Dose Escalation, Dose Expansion,
FE/PK sub-study) is presented in Table 3. Additional assessments for the optional FE/PK sub-
study are presented in Table 4 (FE Cohort) and Table 5 (PK Cohorts 1 and 2).
Phase 1 Dose Escalation
During the Dose Escalation part of the study, dose escalation will follow a Bayesian optimal
interval (BOIN) design. Approximately 6 dose levels are planned for Compound (10b), ranging
from 80 mg to 700 mg. The design will include accelerated titration using a cohort size of 1 for
Cohort 1, as long as no ≥Grade 2 adverse event (AE) is observed in Cohort 1. If any ≥Grade 2
AE is observed in the single-patient cohort (Cohort 1), 2 additional patients will be added to this
dose level. Thereafter, 3-5 patients will be enrolled in each of Cohorts 2 through 6 to have 3
patients qualify for the DLT assessment period (ie, 75% of daily doses taken during Cycle 1) in
each cohort. Patients who do not complete 21 days of IP in Cycle 1 will be replaced, except for
patients who discontinue due to a DLT. At any point, additional patients may be enrolled as
required for safety assessment or as deemed necessary by the SRC.

Planned dose levels for dose escalation are outlined as follows:

| Dose Level/Cohort | Cohort Size (N) ^{a} | Comp. (10b) (Planned Dose) ^{b} |
|---|---|---|
| 1 | 1 | 80 mg QD |
| 2 | 3-5 | 150 mg QD |
| 3 | 3-5 | 250 mg QD |
| 4 | 3-5 | 400 mg QD |
| 5 | 3-5 | 550 mg QD |
| 6* | 3-5 | 700 mg QD |
| 6^{#} | 3-6 | 450 mg QD |

Abbreviations: QD = once daily
*Cohort 6 corresponding to the study design shown in FIG. 9.
^{#}Cohort 6 corresponding to the study design shown in FIG. 14.
^{a} The cohort size assumes accelerated titration at the first dose level;
as many as 6 subjects may be dosed in a given cohort.
^{b} Dose escalation, alternative dose levels or an additional dose level
(e.g., seventh cohort), and/or alternative dose schedules will proceed
following review of the totality of the safety data by the Safety Review
Committee (SRC) and after the SRC approves or makes a recommendation
regarding the next dose cohort.
Note:
Intra-patient dose escalation to the next dose cleared by the SRC may be
permitted after completion of ≥2 cycles for patients who have not experienced
any Grade 3 or higher toxicity during the previous treatment cycle.
With each dose cohort, patients will be monitored for dose-limiting toxicities (DLTs) during the
first cycle of treatment.
For each dose level of Dose Escalation, an SRC will evaluate safety (including DLTs) and
available PK and pharmacodynamic data at the end of the DLT Assessment Period (ie, the first
28 days of treatment [Cycle 1]). The SRC will make dosing and escalation decisions in
accordance with the escalation and de-escalation rules of the BOIN design using a target toxicity
rate of 27% with escalation and de-escalation thresholds of 21.3% and 32.2%, respectively.
Alternative dose levels or an additional dose level (e.g., seventh cohort) and/or alternative dose
schedules may be possible following review of the totality of the data by the SRC. Further, at
any point additional patients may be enrolled in a cohort as required for safety assessment or as
needed by the SRC. The RP2D will be selected after approximately 21 patients are reached or
the number of patients treated at a single dose level reaches 12.
Phase 1b Dose Expansion
Enrollment into the four Dose Expansion cohorts (see FIGS. 10 and 14) will occur
simultaneously and independently of each other. Up to 20 patients are planned for enrollment
into each cohort. Patients will be treated at the RP2D established in Dose Escalation. The dose
may be adjusted based on an SRC decision following review of the totality of the data.
A Simon 2-stage design will be employed for each Dose Expansion cohort to assess preliminary
anti-tumor activity. In the first stage, 10 patients will be enrolled into a cohort. If no responses
are observed in the first 10 patients enrolled in the cohort at the dose decided upon by the SRC, it
will be terminated. If at least 1 response is observed in the first 10 patients, an additional 10
patients will be enrolled into the cohort. If the true response rate is ≥30%, the chance of
observing no responses in the first 10 patients in this design is <2.8%. A true response rate of
30% has statistical power of 76% to rule out a lower inactivity rate of 10% with one-sided type I
error = 5%. Patients with disease and molecular alterations as specified eligible for dose
expansion and treated at the RP2D in either the Dose Escalation part and/or the FE/PK sub-study
may be counted toward the Simon two-stage at the sponsor's discretion.
Optional Food Effect (FE) and PK Sub-study
An optional FE/PK sub-study may be performed. Enrollment into the FE/PK sub-study will
occur simultaneously and independently of Dose Expansion. The FE/PK sub-study will include
up to 3 cohorts. The first cohort (FE Cohort) will include 6-8 patients treated at one dose level
lower than the RP2D dose in Dose Escalation. Two additional cohorts (PK Cohort 1 and PK
Cohort 2) may be enrolled at the sponsor's discretion. PK Cohort 1 and PK Cohort 2 will include
6-8 patients each. Patients in PK Cohort 1 will be treated at the RP2D dose in Dose Escalation;
patients in PK Cohort 2 will be treated at two dose levels lower than the RP2D dose in Dose
Escalation.
Patients enrolled in the FE Cohort of the sub-study will first follow the assessments outlined in
Table 4 (screening and Cycle 0) and then will switch to Table 3 (starting at C1D1 of Dose
Expansion). Patients enrolled in the PK Cohorts of the sub-study will first follow the assessments
outlined in Table 5 (screening and Cycle 0) and then will switch to Table 3 (starting at C1D1 of
Dose Expansion).
During Cycle 0 of the FE/PK sub-study, patients in the FE Cohort will be given a single dose of
Compound (10b) after an overnight fast followed by safety, PK, and pharmacodynamic
assessments. After a 7-day washout (from time of dosing), patients will be given a single dose of
Compound (10b) after a high-fat/high-calorie breakfast followed by safety, PK, and
pharmacodynamic assessments. After a 3-day washout, patients will continue with treatment at
the RP2D established in Dose Escalation (starting at C1D1), or as modified based on an SRC
decision following review of the totality of the data.
Patients in the PK Cohorts will be given a single dose of Compound (10b) after an overnight fast
followed by safety, PK, and pharmacodynamic assessments. After a 7-day washout (from time of
dosing), patients will continue with treatment at the RP2D established in Dose Escalation
(starting at C1D1), or as modified based on an SRC decision following review of the totality of
the data.
Test Product, Dose, and Administration: Starting at C1D1, IP (Compound (10b)) will be taken
once daily (QD) in each 28-day treatment cycle; it will be administered as oral capsules of 10, 50
and/or 100 mg. Dosing in Cycle 0 of the FE/PK sub-study is outlined above.
Reference Therapy, Dose, and Administration: None
CRITERIA FOR EVALUATION:
Endpoints:
Primary Endpoints
Incidence and severity of treatment-emergent AEs (TEAEs), serious AEs (SAEs), and DLTs
Secondary Endpoints
Abnormalities in clinical laboratory parameters, vital sign measurements, and ECGs
Antitumor activity, as assessed by objective response rate (ORR, complete response + partial
response rate), duration of response (DOR), and progression free survival (PFS)
Plasma PK parameters of Compound (10b) (including, but not limited to: AUC, Cmax,
Tmax, estimated half-life) from plasma concentration-time data under fasted and fed
conditions
Pharmacodynamic parameters
Exploratory Endpoints
Biomarkers that correlate with response to Compound (10b)
Urinary excretion parameters of Compound (10b)
STATISTICAL CONSIDERATIONS: Descriptive statistics will be employed to analyze the
data. Summary statistics for continuous variables will include the mean, standard deviation,
median, and range (minimum/maximum); categorical variables will be presented as frequency
counts and percentages.
Pharmacokinetic parameters will be determined based on individual plasma concentration-time
profiles using noncompartmental methods. For all parts of the study, AEs will be coded using the
Medical Dictionary for Regulatory Activities by preferred term and system organ class. The
maximum severity and frequency will be summarized. Treatment-emergent AEs will be
tabulated by severity grade and relationship to IP. Also, TEAEs leading to permanent
discontinuation of IP and those leading to death or study withdrawal will be listed and
summarized. Efficacy data (e.g., ORR) will be summarized using descriptive statistics and
presented by dose level for the dose escalation part of the study and by disease type for the dose
expansion part of the study. Time to event endpoints (e.g., PFS) will be summarized by tumor
type for the dose expansion part of the study using Kaplan-Meier method estimates when
appropriate. No formal interim analysis is planned.
The primary analysis will be conducted after the last patient in Dose Expansion completes either
6 cycles on IP, discontinues IP, or is withdrawn from the study.

TABLE 3
Sample Schedule of Assessments

| | | | | | | | | Subsequent | Subsequent | Early | EOT | Long-Term |
|---|---|---|---|---|---|---|---|---|---|---|---|---|
| | | | | | | | | ODD Cycles | EVEN Cycles | Termination | Follow- | Follow- |
| | Screen^{aa} | Cycle 1^{ee} | | | | Cycle 2 | | (eg, 3, 5) | (eg, 4, 6) | (ET) | up | up |
| | Study Day | | | | | | | | | | | |
| | −30 | | | | | | | | | | LD + | Every 3 |
| | to −1 | 1^{w} | 2 | 8 | 15 | 1^{v, cc} | 15 | 1^{v, cc} | 1^{v, cc} | (n/a) | 28 | months |
| | ±days allowable | | | | | | | | | | | |
| | 0 | 0 | 0 | ±3 | ±3 | ±3^{x} | ±3 | ±3 | ±3 | (n/a) | ±3 | NA |
| Dosing | | | | | | | | | | | | |
| IP Administration^{u} | | X | X | X | X | X | X | X | X | | | |
| Disease Assessment | | | | | | | | | | | | |
| Restaging scan (CT, | X | | | | | | | X (±7 d) | | | X (±7 | |
| MRI, or PET-CT)^{y} | | | | | | | | | | | d)^{ff} | |
| Relevant tumor | X | | | | | | | X | | | X | |
| marker^{z} | | | | | | | | | | | | |
| DLT Assessment | | | | | | | | | | | | |
| Period (Dose | | | | | | | | | | | | |
| Escalation Only) | | | | | | | | | | | | |
| See DLT definition | | X-----------------------------------------X | | | | | | | | | | |
| below | | | | | | | | | | | | |
| Study Procedures & | | | | | | | | | | | | |
| Examinations^{aa} | | | | | | | | | | | | |
| Eligibility | X | X^{j} | | | | | | | | | | |
| Assessment & | | | | | | | | | | | | |
| Informed Consent^{j} | | | | | | | | | | | | |
| Demographics^{k} | X | | | | | | | | | | | |
| Medical History^{l} | X | X^{h} | | | | | | | | | | |
| 12-lead ECG | | | | | | | | | | | | |
| (triplicate) (QTcF) | | | | | | | | | | | | |
| Dose Escalation/ | X | X^{f} | X^{b} | | | x^{f} | | X^{a} | | X | X | |
| Expansion | | | | | | | | | | | | |
| Dose Expansion | | | | | | X^{f} | | X^{a} | | X | X | |
| following FE/PK | | | | | | | | | | | | |
| sub-study Cycle 0 | | | | | | | | | | | | |
| MUGA/ECHO | X | | | | | X^{bb} | | X^{bb} | | X^{bb} | X^{bb} | |
| Physical Exam^{m,n} | X | X^{h} | | X | | X | | X | X | X | X | |
| ECOG Performance | X | Xⁱ | | | | X | | X | X | X | X | |
| Status | | | | | | | | | | | | |
| Vital Signs | X | X^{a} | X^{b} | X^{a} | | X^{a} | | X^{a} | X^{a} | X | X | |
| Prior/Concomitant | Document any medication or vaccine that the patient is receiving at the time of signing the ICF | | | | | | | | | | | Document |
| Medications | and/or within 30 d or 5 half-lives (whichever is longer) of first dose of IP. Document concomitant | | | | | | | | | | | subsequent |
| | medications taken from time of signing the ICF through 28 d after the last dose of IP or date of the | | | | | | | | | | | anticancer |
| | decision to permanently discontinue study treatment. | | | | | | | | | | | treatment |
| AEs ^{o} | Document AEs from first dose of IP through at least 28 d after the last dose of IP or date of the | | | | | | | | | | | |
| | decision to permanently discontinue study treatment. AE information may be collected at study | | | | | | | | | | | |
| | visits or over the phone | | | | | | | | | | | |
| Survival | | | | | | | | | | | | X^{gg} |
| Laboratory | | | | | | | | | | | | |
| Evaluations^{aa} | | | | | | | | | | | | |
| FSH^{p} | X | | | | | | | | | | | |
| Pregnancy test^{q} | X | | | X^{a} | | X^{a} | | | | X | X | |
| HIV, HBV & HCV | X | | | | | | | | | | | |
| Blood Hematology^{r} | X | X^{a, aa} | X^{b} | X^{a} | X^{a} | X^{a} | X^{a} | X^{a} | X^{a} | X | X | |
| Chemistry^{s} | X | X^{a, aa} | X^{b} | X^{a} | X^{a} | X^{a} | X^{a} | X^{a} | X^{a} | X | X | |
| Coagulation (aPTT, | | | | | | | | | | | | |
| PT, INR) | | | | | | | | | | | | |
| Dose Escalation/ | X | X^{c, aa} | X^{b} | | | X^{c} | | x^{a} | X^{a} | X | X | |
| Expansion | | | | | | | | | | | | |
| Dose Expansion | | | | | | X^{c} | | X^{a} | X^{a} | X | X | |
| following FE/PK | | | | | | | | | | | | |
| sub-study Cycle 0 | | | | | | | | | | | | |
| Urinalysis (spot | X | X^{a, aa} | | | | X^{a} | | X^{a} | X^{a} | X | X | |
| collection) | | | | | | | | | | | | |
| Research Studies | | | | | | | | | | | | |
| (Pharmacodynamics/PK) | | | | | | | | | | | | |
| PK - Blood samples | | | | | | | | | | | | |
| Dose Escalation/ | X | X^{d} | X^{b} | X^{a} | X^{a} | X^{d} | | X^{g} | X^{g} | | | |
| Expansion^{ee} | | | | | | | | | | | | |
| Dose Expansion | | X^{a} | | X^{a} | X^{a} | X^{d} | | X^{g} | X^{g} | | | |
| following FE/PK | | | | | | | | | | | | |
| sub-study Cycle 0 | | | | | | | | | | | | |
| Pharmacodynamic | | | | | | | | | | | | |
| Biomarker - | | | | | | | | | | | | |
| Blood/PBMC | | | | | | | | | | | | |
| samples | | | | | | | | | | | | |
| Dose Escalation/ | | X^{f} | X^{a} | | | X^{f} | | X^{g} | X^{g} | | X | |
| Expansion | | | | | | | | | | | | |
| Dose Expansion | | | | | | X^{f} | | X^{g} | X^{g} | | X | |
| following FE/PK | | | | | | | | | | | | |
| sub-study Cycle 0 | | | | | | | | | | | | |
| Circulating tumor | X | | | | | X | | X | X | | X | |
| markers^{t} | | | | | | | | | | | | |
| Fresh tumor biopsy | X^{dd} | | | | | X^{e, dd} | | | | | X^{e, dd} | |

Abbreviations and Footnotes of Table 3
Abbreviations: AE = adverse event; ALT/SGPT = alanine aminotransferase/serum glutamic-pyruvic transaminase; aPTT = activated partial thromboplastin time; AST/SGOT = aspartate aminotransferase/serum glutamic-oxaloacetic transaminase; CA125 = cancer antigen 125; CEA = carcinoembryonic antigen; cfDNA = circulating free deoxyribonucleic acid; CPK = creatine phosphokinase; CR = complete response(s); CT = computed tomography; DLT = dose-limiting toxicity; ECG = electrocardiogram; ECHO = echocardiogram; ECOG = Eastern Cooperative Oncology Group; EOT = End of treatment; ET = Early Termination; FE = food effect; FSH = follicle stimulating hormone; HBV = hepatitis B virus; HCV = hepatitis C virus; HIV = human immunodeficiency virus; ICF = informed consent form; INR = international normalized ratio; IP = investigational product; LD = last dose; MRI = magnetic resonance imaging; MUGA = multiple-gated acquisition scan; NA = not applicable; PBMC = peripheral blood mononuclear cell; PET = positron emission tomography; PK = pharmacokinetic; PR = partial response; PSA = prostate specific antigen; PT = prothrombin time; QTcF = QT using Fridericia's correction formula; SAE = serious adverse event; SRC = safety review committee.
^{a}Collected pre-dose.
^{b}Collected pre-dose, that is approximately 24 hr (±3 hr) after prior day's IP administration.
^{c}Collected pre-dose and 4 hr (all timepoints ±15 min) after IP administration. See footnote aa for additional timing on the pre-dose assessment.
^{d}Collected pre-dose, 0.5 (±5 min), 1, 2, 4 and 6 hr (all timepoints ±15 min unless otherwise specified) after IP administration.
^{e}Collect post-dose between 2 and 6 hr after administration of IP on Cycle 2 Day 1 (±7 d depending on the scheduling availability at the center) and at the EOT Follow-up Visit (±7 d).
^{f}Collected pre-dose, 2 and 4 hr (all timepoints ±15 min) after IP administration.
^{g}Collected pre-dose and 2 hr (all timepoints ±15 min) after IP administration.
^{h}Medical history and physical exam should be confirmed and updated with any findings which may have occurred prior to the first dose on C1D1.
ⁱUnless done in the prior 7 d.
^{j}Eligibility to be confirmed prior to dosing.
^{k}Including year of birth, sex, height, race, ethnicity.
^{l}Including relevant medical history, current medical conditions, oncology history (eg, diagnosis, extent of cancer, prior anticancer therapy), radiation history.
^{m}Complete physical exam to be performed at Screening and EOT/ET; abbreviated exam (eg, symptom directed per investigator's judgment) to be performed at other visits; includes weight for both complete and abbreviated exams.
ⁿAdverse events per patient history will be collected at time of physician examination.
^{o} Serious AEs should be collected from time of informed consent. Death solely due to progression of disease should not be reported as an SAE but as with all deaths, must be captured on the relevant CRF page.
^{p}Women who are not surgically sterile to confirm post-menopausal state.
^{q}Women who are not surgically sterile or confirmed post-menopausal; serum test at all time points.
^{r}Complete blood count, differential, platelets.
^{s}Glucose, total protein, albumin, electrolytes [sodium, potassium, chloride, total CO₂], calcium, phosphorus, magnesium, uric acid, bilirubin (total, direct), ALT/SGPT, AST/SGOT, alkaline phosphatase, creatinine, blood urea nitrogen, CPK, cholesterol, lactate dehydrogenase [NOTE: labs should be done fasting for screening labs and fasting for all pre-dose labs].
^{t}For example, cfDNA.
^{u}IP will be administered once daily for a 28-d cycle, and in a similar manner in all cycles, unless an alternative dosing schedule is implemented based on SRC review. IP should be taken after an overnight fast of at least 8 hr and followed by 2 hr of fasting after the dose is taken.
^{v}If a dose interruption occurs at the end of a cycle and IP is not restarted prior to the first day of the next cycle, the first day of the next cycle will be the day the IP is restarted. Should IP be interrupted for 15 consecutive days, the patient should be discontinued permanently from IP.
^{w}C1D1 visit should take place on a Monday, Tuesday, Wednesday, or Thursday.
^{x}To qualify for the DLT assessment period, patients need to have taken 21 doses within the 28-day assessment period.
^{y}Restaging scans (CT, MRI, or PET-CT) will be done every 2 cycles (ie, every 8 wk). Confirmatory scans will also be obtained at least 4 wk following initial documentation of an objective response (ie, PR or CR). The type of scan obtained is at the discretion of the investigator as appropriate for the disease. However, the same method should be used for the duration of the study. Window (C3D1 ±7 d and subsequent cycles ±7 d) allowed for disease scan assessments. All scans will be read locally.
^{z}Relevant tumor markers (eg, CA125, PSA, CEA) will be assessed once every 2 cycles after completion of the first 2 cycles, following the same schedule as restaging scans, as appropriate.
^{aa}Blood hematology, chemistry, coagulation & urinalysis pre-dose assessments may be performed up to 2 d prior to scheduled visit. If the screening assessments are performed within 48 hr prior to C1D1, these results may be used as baseline (pre-dose assessments) without requiring a repeat assessment.
^{bb}Window (±3 d) allowed for each MUGA/ECHO evaluation starting at Cycle 2.
^{cc}If patient will not be continuing on subsequent cycle (ie, not continuing on study), patient should still undergo all Day 1 visit procedures except the 2 hr PK and pharmacodynamic samples.
^{dd}Fresh tumor biopsies should be collected for all patients enrolled in the study, when feasible (screening, Cycle 2 Day 1 [±7d] and EOT Follow-up Visit [±7d]). When fresh tumor biopsies are not feasible during screening, archival tumor biopsies collected within 1 year of enrollment can be used. Patients may be enrolled even when tumor biopsies cannot be obtained at the discretion of the medical monitor, in consultation with the investigator. Enrollment of patients for whom collection of tumor biopsies will not occur will be handled on a case-by-case basis by the medical monitor. Please refer to the Lab Specimen Manual for details regarding collection and handling of tumor tissue samples.
^{ee}Patients undergoing intra-patient dose escalation will undergo Cycle 1 assessment schedule during first cycle at the new dose level, but without PK draws.
^{ff}Unless completed within 4 wk of the EOT Follow-up Visit.
^{gg}In addition to survival data, long-term follow up will include the collection of disease response(s) and secondary malignancies.

TABLE 4
Sample Schedule of Assessments for Optional FE/PK Sub-study: FE Cohort

| | Screen | Treatment | | | | | | | | | | | | | | | | | | | | | |
|---|---|---|---|---|---|---|---|---|---|---|---|---|---|---|---|---|---|---|---|---|---|---|---|
| | Cycle | | | | | | | | | | | | | | | | | | | | | | |
| | | 0 | | | | | | | | | | | | | | | | | | | | | |
| | Day | | | | | | | | | | | | | | | | | | | | | | |
| | −30 | | | | | | | | | | | | | | | | | | | | | | |
| | to −1 | 1 | | | | | | | | 2 | 3 | 4 | 8 | | | | | | | | 9 | 10 | Cycle 0 |
| | Hours (relative to dosing)^{b} | | | | | | | | | | | | | | | | | | | | | | Day 11 |
| | | 0 | 0.5 | 1 | 2 | 3 | 4 | 6 | 8 | 24 | 48 | 72 | 0 | 0.5 | 1 | 2 | 3 | 4 | 6 | 8 | 24 | 48 | (−1 d/+3 d) |
| Dosing | | | | | | | | | | | | | | | | | | | | | | | Proceed |
| IP Administration^{b} | | X^{t} | | | | | | | | | | | X^{u} | | | | | | | | | | to Cycle |
| Disease Assessment | | | | | | | | | | | | | | | | | | | | | | | 1 Day 1 |
| Restaging scan (CT, | X | | | | | | | | | | | | | | | | | | | | | | of Dose |
| MRI, or PET-CT) | | | | | | | | | | | | | | | | | | | | | | | Expansion^{a} |
| Relevant tumor | X | | | | | | | | | | | | | | | | | | | | | | |
| marker (eg, CA125, | | | | | | | | | | | | | | | | | | | | | | | |
| PSA, CEA) | | | | | | | | | | | | | | | | | | | | | | | |
| Study Procedures & | | | | | | | | | | | | | | | | | | | | | | | |
| Examinations | | | | | | | | | | | | | | | | | | | | | | | |
| Eligibility | X | X^{f} | | | | | | | | | | | | | | | | | | | | | |
| Assessment & | | | | | | | | | | | | | | | | | | | | | | | |
| Informed Consent^{f} | | | | | | | | | | | | | | | | | | | | | | | |
| Demographics^{g} | X | | | | | | | | | | | | | | | | | | | | | | |
| Medical history^{h} | X | X^{k} | | | | | | | | | | | | | | | | | | | | | |
| 12-lead ECG | X | X^{r} | | | X | | X | | | | | | X^{r} | | | X | | X | | | | | |
| (triplicate) (QTcF) | X | | | | | | | | | | | | | | | | | | | | | | |
| MUGA/ECHO | | | | | | | | | | | | | | | | | | | | | | | |
| Physical Exam^{i,j} | X | X^{k,r} | | | | | | | | | | | | | | | | | | | | | |
| ECOG Performance | X | X^{d,r} | | | | | | | | | | | | | | | | | | | | | |
| Status | | | | | | | | | | | | | | | | | | | | | | | |
| Vital Signs | X | X^{r} | | | | | | | | | | | | | | | | | | | | | |
| Prior/Concomitant | Document any medication or vaccine that the patient is receiving at the time of signing the ICF and/or | | | | | | | | | | | | | | | | | | | | | | |
| Medications | within 30 d or 5 half-lives (whichever is longer) of first dose of IP. Document concomitant | | | | | | | | | | | | | | | | | | | | | | |
| | medications taken from time of signing the ICF through 28 d after the last dose of IP or date of the | | | | | | | | | | | | | | | | | | | | | | |
| | decision to permanently discontinue study treatment. | | | | | | | | | | | | | | | | | | | | | | |
| AEs^{l} | Document AEs from first dose of IP through at least 28 d after the last dose of IP or date of the | | | | | | | | | | | | | | | | | | | | | | |
| | decision to permanently discontinue study treatment. AE information may be collected at study visits | | | | | | | | | | | | | | | | | | | | | | |
| | or over the phone. | | | | | | | | | | | | | | | | | | | | | | |
| Laboratory | | | | | | | | | | | | | | | | | | | | | | | Proceed |
| Evaluations | | | | | | | | | | | | | | | | | | | | | | | to Cycle |
| FSH^{m} | X | | | | | | | | | | | | | | | | | | | | | | 1 Day 1 |
| Pregnancy testⁿ | X | | | | | | | | | | | | | | | | | | | | | | of Dose |
| HIV, HBV & HCV | X | | | | | | | | | | | | | | | | | | | | | | Expansion^{a} |
| Blood Hematology^{o} | X | X^{r,v} | | | | | | | | X^{s} | | | | | | | | | | | | | |
| Chemistry^{p} | X | X^{r,v} | | | | | | | | X^{s} | | | | | | | | | | | | | |
| Coagulation (aPTT, | X | X^{w,v} | | | | | X | | | X^{s} | | | | | | | | | | | | | |
| PT, INR) | | | | | | | | | | | | | | | | | | | | | | | |
| Urinalysis (spot | X | X^{r,v} | | | | | | | | | | | | | | | | | | | | | |
| collection) | | | | | | | | | | | | | | | | | | | | | | | |
| Research Studies | | | | | | | | | | | | | | | | | | | | | | | |
| (Pharmacodynamic/ | | | | | | | | | | | | | | | | | | | | | | | |
| PK) | | | | | | | | | | | | | | | | | | | | | | | |
| Comp. (10b) PK^{x} | | X^{r} | X | X | X | X | X | X | X | X | X | X | X | X | X | X | X | X | X | X | X | X | |
| Pharmacodynamic | | X^{r} | | | X | | X | | | | | | X | | | X | | X | | | | | |
| Biomarker - | | | | | | | | | | | | | | | | | | | | | | | |
| Blood/PBMC | | | | | | | | | | | | | | | | | | | | | | | |
| samples^{x} | | | | | | | | | | | | | | | | | | | | | | | |
| Circulating tumor | X | | | | | | | | | | | | | | | | | | | | | | |
| markers^{q} | | | | | | | | | | | | | | | | | | | | | | | |
| Fresh tumor biopsy | X^{e} | | | | | | | | | | | | | | | | | | | | | | |
| Renal Excretion | | | | | | | | | | | | | | | | | | | | | | | |
| Assessment | | | | | | | | | | | | | | | | | | | | | | | |

| | | | | |
|---|---|---|---|---|
| Urine^{c} | X | X | X | X |

Abbreviations and Footnotes of Table 4
Abbreviations:
AE = adverse event;
ALT/SGPT = alanine aminotransferase/serum glutamic-pyruvic transaminase;
aPTT = activated partial thromboplastin time;
AST/SGOT = aspartate aminotransferase/serum glutamic-oxaloacetic transaminase;
CA125 = cancer antigen 125;
CEA = carcinoembryonic antigen;
cfDNA = circulating free deoxyribonucleic acid;
CPK = creatine phosphokinase;
CT = computed tomography;
ECG = electrocardiogram;
ECHO = echocardiogram;
ECOG = Eastern Cooperative Oncology Group;
EOT = End of treatment;
ET = Early Termination;
FSH = follicle stimulating hormone;
HBV = hepatitis B virus;
HCV = hepatitis C virus;
HIV = human immunodeficiency virus;
ICF = informed consent form;
INR = international normalized ratio;
IP = investigational product;
LD = last dose;
MRI = magnetic resonance imaging;
MUGA = multiple-gated acquisition scan;
PET = positron emission tomography;
PK = pharmacokinetic;
PSA = prostate specific antigen;
PT = prothrombin time;
QTcF = QT using Fridericia's correction formula
^{a}Refer to Table 3 for assessments and study continuation for Dose Expansion. Window to initiate C1D1 dose at the RP2D: LD + 2 up to 5 d (2 d washout + up to additional 3 d).
^{b}At C0D1, patients will be administered Compound (10b) in a fasted state (no food minimum 8 hr before dosing and 2 hr after dosing). At C0D8, patients will be administered Compound (10b) in a fed state after high-fat/high-calorie meal. During Dose Expansion (ie, starting at C1D1), Compound (10b) will be administered in a fasted state.
^{c}Urine for renal excretion assessment should be collected 0-8 hr post dose, 8-24 hr post dose, 24-48 hr post dose, and 48-72 hr post dose.
^{d}Unless done in the prior 7 d.
^{e}Fresh tumor biopsies should be collected for all patients enrolled in the study, when feasible. When fresh tumor biopsies are not feasible during screening, archival tumor biopsies collected within 1 year of enrollment can be used. Patients may be enrolled even when tumor biopsies cannot be obtained at the discretion of the medical monitor, in consultation with the investigator. Enrollment of patients for whom collection of tumor biopsies will not occur will be handled on a case-by-case basis by the medical monitor. Please refer to the Lab Specimen Manual for details regarding collection and handling of tumor tissue samples.
^{f}Including relevant medical history, current medical conditions, oncology history (eg, diagnosis, extent of cancer, prior anticancer therapy), radiation history.
^{g}Including date of birth, sex, height, race, ethnicity.
^{h}Including oncology history, radiation history.
ⁱComplete physical exam to be performed at Screening and EOT/ET; abbreviated exam (eg, symptom directed per investigator's judgment) to be performed at other visits; includes weight for both complete and abbreviated exams.
^{j}Adverse events per patient history will be collected at time of physician examination.
^{k}Medical history and physical exam should be confirmed and updated with any findings which may have occurred prior to the first dose on C0D1.
^{l}Serious AEs should be collected from time of informed consent. Death solely due to progression of disease should not be reported as an SAE but as with all deaths, must be captured on the relevant CRF page.
^{m}Women who are not surgically sterile to confirm post-menopausal state.
ⁿWomen who are not surgically sterile or confirmed post-menopausal; serum test at all time points.
^{o}Complete blood count, differential, platelets.
^{p}Glucose, total protein, albumin, electrolytes [sodium, potassium, chloride, total CO₂], calcium, phosphorus, magnesium, uric acid, bilirubin (total, direct), ALT/SGPT, AST/SGOT, alkaline phosphatase, creatinine, blood urea nitrogen, CPK, cholesterol, lactate dehydrogenase [NOTE: labs should be done fasting for screening labs and fasting for all pre-dose labs].
^{q}For example, cfDNA.
^{r}Collected pre-dose.
^{s}Collected pre-dose approximately 24 hr (±3 hr) after prior day's IP administration.
^{t}Patients will be administered Compound (10b) in a fasted state (no food minimum 8 hr before dosing and 2 hr after dosing).
^{u}Patients will be administered Compound (10b) in a fed state after high-fat/high-calorie meal.
^{v}Blood hematology, chemistry, coagulation & urinalysis pre-dose assessments may be performed up to 2 d prior to scheduled visit. If the screening assessments are performed within 48 hr prior to C0D1, these results may be used as baseline (pre-dose assessments) without requiring a repeat assessment.
^{w}Collected pre-dose and 4 hr (all timepoints ±15 min) after IP administration. See footnote v for additional timing on the pre-dose assessment.
^{x}Time windows for sampling post dose (as applicable): 0.5 hr (±5 min); 1 hr to 8 hr time points (±15 min); 24 hr to 72 hr time points (±3 hr).

TABLE 5
Sample Schedule of Assessments for Optional FE/PK Sub-study: PK Cohorts 1 and 2

| | Screen | Treatment | | | | | | | | | | | | | | |
|---|---|---|---|---|---|---|---|---|---|---|---|---|---|---|---|---|
| | Cycle | | | | | | | | | | | | | | | |
| | | 0 | | | | | | | | | | | | | | |
| | Day | | | | | | | | | | | | | | | |
| | −30 to −1 | 1 | | | | | | | | 2 | 3 | 4 | 5 | 6 | 7 | Cycle 0 |
| | Hours (relative to dosing)^{b} | | | | | | | | | | | | | | | Day 8 |
| | | 0 | 0.5 | 1 | 2 | 3 | 4 | 6 | 8 | 24 | 48 | 72 | | | | (−1 d/+3 d) |
| Dosing | | | | | | | | | | | | | | | | Proceed |
| IP Administration | | X ^{c} | | | | | | | | | | | | | | to Cycle |
| Disease Assessment | | | | | | | | | | | | | | | | 1 Day1 |
| Restaging scan (CT, MRI, or | X | | | | | | | | | | | | | | | of Dose |
| PET-CT) | | | | | | | | | | | | | | | | Expansion^{a} |
| Relevant tumor marker (eg, | X | | | | | | | | | | | | | | | |
| CA125, PSA, CEA) | | | | | | | | | | | | | | | | |
| Study Procedures & | | | | | | | | | | | | | | | | |
| Examinations | | | | | | | | | | | | | | | | |
| Eligibility Assessment & | X | X^{f} | | | | | | | | | | | | | | |
| Informed Consent^{f} | | | | | | | | | | | | | | | | |
| Demographics^{g} | X | | | | | | | | | | | | | | | |
| Medical history^{h} | X | X^{r} | | | | | | | | | | | | | | |
| 12-lead ECG (triplicate) | X | X^{q} | | | X | | X | | | | | | | | | |
| (QTcF) | | | | | | | | | | | | | | | | |
| MUGA/ECHO | X | | | | | | | | | | | | | | | |
| Physical Exam^{i,j} | X | X^{q,r} | | | | | | | | | | | | | | |
| ECOG Performance Status | X | X^{q,t} | | | | | | | | | | | | | | |
| Vital Signs | X | X^{q} | | | | | | | | | | | | | | |
| Prior/Concomitant | Document any medication or vaccine that the patient is receiving at the time of signing the | | | | | | | | | | | | | | | |
| Medications | ICF and/or within 30 d or 5 half-lives (whichever is longer) of first dose of IP. Document | | | | | | | | | | | | | | | |
| | concomitant medications taken from time of signing the ICF through 28 d after the last dose | | | | | | | | | | | | | | | |
| | of IP or date of the decision to permanently discontinue study treatment. | | | | | | | | | | | | | | | |
| AEs^{k} | Document AEs from first dose of IP through at least 28 d after the last dose of IP or date of | | | | | | | | | | | | | | | Proceed |
| | the decision to permanently discontinue study treatment. AE information may be collected at | | | | | | | | | | | | | | | to Cycle |
| | study visits or over the phone. | | | | | | | | | | | | | | | 1 Day 1 |
| Laboratory Evaluations | | | | | | | | | | | | | | | | of Dose |
| FSH^{l} | X | | | | | | | | | | | | | | | Expansion^{a} |
| Pregnancy test^{m} | X | | | | | | | | | | | | | | | |
| HIV, HBV & HCV | X | | | | | | | | | | | | | | | |
| Blood Hematologyⁿ | X | X^{q,s} | | | | | | | | X^{u} | | | | | | |
| Chemistry^{o} | X | X^{q,s} | | | | | | | | X^{u} | | | | | | |
| Coagulation (aPTT, PT, INR) | X | X^{v,s} | | | | | X | | | X^{u} | | | | | | |
| Urinalysis (spot collection) | X | X^{q,s} | | | | | | | | | | | | | | |
| Research Studies | | | | | | | | | | | | | | | | |
| (Pharmacodynamic/PK) | | | | | | | | | | | | | | | | |
| Compound (10b) PK^{b} | | X | X | X | X | X | X | X | X | X | X | X | | | | |
| Pharmacodynamic Biomarker - | | X | | | X | | X | | | | | | | | | |
| Blood/PBMC samples | | | | | | | | | | | | | | | | |
| Circulating tumor markers^{p} | X | | | | | | | | | | | | | | | |
| Fresh tumor biopsy | X^{e} | | | | | | | | | | | | | | | |
| Renal Excretion | | | | | | | | | | | | | | | | |
| Assessment | | | | | | | | | | | | | | | | |

| | | | | |
|---|---|---|---|---|
| Urine ^{d} | X | X | X | X |

Abbreviations and Footnotes of Table 5
Abbreviations:
AE = adverse event;
ALT/SGPT = alanine aminotransferase/serum glutamic-pyruvic transaminase;
aPTT = activated partial thromboplastin time;
AST/SGOT = aspartate aminotransferase/serum glutamic-oxaloacetic transaminase;
CA125 = cancer antigen 125;
CEA = carcinoembryonic antigen;
cfDNA = circulating free deoxyribonucleic acid;
CPK = creatine phosphokinase;
CT = computed tomography;
ECG = electrocardiogram;
ECHO = echocardiogram;
ECOG = Eastern Cooperative Oncology Group;
FSH = follicle stimulating hormone;
HBV = hepatitis B virus;
HCV = hepatitis C virus;
HIV = human immunodeficiency virus;
ICF = informed consent form;
INR = international normalized ratio;
IP = investigational product;
LD = last dose;
MRI = magnetic resonance imaging;
MUGA = multiple-gated acquisition scan;
PET = positron emission tomography;
PK = pharmacokinetic;
PSA = prostate specific antigen;
PT = prothrombin time;
QTcF = QT using Fridericia's correction formula
^{a}Refer to Table 3 for assessments and study continuation for Dose Expansion. Window to initiate C1D1 dose at the RP2D: LD + 5 up to 10 d.
^{b}Time windows for sampling post dose: 0.5 hr (±5 min); 1 hr to 8 hr time points (±15 min); 24 hr to 72 hr time points (±3 hr).
^{c} Patients will be administered Compound (10b) in a fasted state (no food minimum 8 hr before dosing and 2 hr after dosing).
^{d} Urine for renal excretion assessment should be collected 0-8 hr post dose, 8-24 hr post dose, 24-48 hr post dose, and 48-72 hr post dose.
^{e}Fresh tumor biopsies should be collected for all patients enrolled in the study, when feasible. When fresh tumor biopsies are not feasible during screening, archival tumor biopsies collected within 1 year of enrollment can be used. Patients may be enrolled even when tumor biopsies cannot be obtained at the discretion of the medical monitor, in onsultation with the investigator. Enrollment of patients for whom collection of tumor biopsies will not occur will be handled on a case-by-case basis by the medical monitor. Please refer to the Lab Specimen Manual for details regarding collection and handling of tumor tissue samples.
^{f}Eligibility to be confirmed prior to dosing.
^{g}Including date of birth, sex, height, race, ethnicity.
^{h}Including relevant medical history, current medical conditions, oncology history (eg, diagnosis, extent of cancer, prior anticancer therapy), radiation history.
ⁱComplete physical exam to be performed at Screening and EOT/ET; abbreviated exam (eg, symptom directed per investigator's judgment) to be performed at other visits; includes weight for both complete and abbreviated exams.
^{j}Adverse events per patient history will be collected at time of physician examination.
^{k}Serious AEs should be collected from time of informed consent. Death solely due to progression of disease should not be reported as an SAE but as with all deaths, must be captured on the relevant CRF page.
^{l}Women who are not surgically sterile to confirm post-menopausal state.
^{m}Women who are not surgically sterile or confirmed post-menopausal; serum test at all time points.
ⁿComplete blood count, differential, platelets.
^{o}Glucose, total protein, albumin, electrolytes [sodium, potassium, chloride, total CO₂], calcium, phosphorus, magnesium, uric acid, bilirubin (total, direct), ALT/SGPT, AST/SGOT, alkaline phosphatase, creatinine, blood urea nitrogen, CPK, cholesterol, lactate dehydrogenase [NOTE: labs should be done fasting for screening labs and fasting for all pre-dose labs].
^{p}For example, cfDNA.
^{q}Collected pre-dose.
^{r}Medical history and physical exam should be confirmed and updated with any findings which may have occurred prior to the first dose on C0D1.
^{s}Blood hematology, chemistry, coagulation & urinalysis pre-dose assessments may be performed up to 2 d prior to scheduled visit. If the screening assessments are performed within 48 hr prior to C0D1, these results may be used as baseline (pre-dose assessments) without requiring a repeat assessment.
^{t}Unless done in the prior 7 d.
^{u}Collected pre-dose approximately 24 hr (±3 hr) after prior day's IP administration.
^{v}Collected pre-dose and 4 hr (all timepoints ±15 min) after IP administration. See footnote s for additional timing on the pre-dose assessment.

Dose-limiting toxicity (DLT) is defined as an AE or abnormal laboratory value, excluding toxicities clearly related to disease progression or intercurrent illness, and occurring during the first cycle (28 days) on study that meets any of the following criteria:
Grade 3 lasting more than 7 days or Grade 4 laboratory abnormalities of aspartate amino transferase/alanine amino transferase (AST/ALT) and/or bilirubin, of any duration, with the following exceptions:
   For patients with Grade 1 AST/ALT at baseline (>ULN to 3×ULN), an AST/ALT value of >7.5×ULN will be considered a DLT.
   For patients with Grade 2 AST/ALT at baseline (>3×ULN to >5×ULN), an AST/ALT value >10×ULN will be considered a DLT.
Grade 3 or higher non-hematological toxicity excluding:
   Grade 3 nausea, vomiting or diarrhea for less than 72 hours with adequate supportive care.
   Grade 3 fatigue lasting less than a week.
   Grade 3 electrolyte abnormality that lasts for less than 72 hours that is not clinically complicated and resolves spontaneously or with conventional medical interventions.
   Grade 3 amylase or lipase lasting less than 72 hours and not associated with manifestations of pancreatitis.
Meet criteria for Hy's Law (ie, ≥3×ULN ALT and/or AST with ≥2×ULN total bilirubin and alkaline phosphatase <2×ULN), unless patient has congenital hyperbilirubinemia.
Grade 3 neutropenia >7 days.
Grade 4 neutropenia.
Febrile neutropenia [ANC <1000/mm³ with single oral temperature >38.3° C. (101° F.) or with two consecutive oral temperatures ≥38° C. (100.4° F.) measured at least 1 hour apart].
Grade 3 thrombocytopenia with Grade 2 or higher bleeding.
Grade 4 thrombocytopenia or thrombocytopenia requiring platelet transfusion.
Grade 4 life-threatening anemia.
Grade 5 toxicities.
Any other ≥Grade 3 clinically significant or persistent toxicity that may be considered a DLT following review by the SRC.

APPENDIX 1
Allowable MAPK-Pathway Alterations

| | |
|---|---|
| KRAS | CCKR |
| NRAS | NGFR |
| HRAS | HGFR |
| CRAF | EphR |
| BRAF (excluding V600X) | AXLR |
| NRAF | TIE receptor |
| MAPK/ERK | RYK receptor |
| MAPKK/MEK | DDR receptor |
| NF1 | RET receptor |
| EGFR | ROS receptor |
| IGFR | LTK receptor |
| PDGFR | ROR receptor |
| VEGFR | MuSK receptor |
| FGFR | |

APPENDIX 2
ECOG Performance Status

| Grade | ECOG Performance Status |
|---|---|
| 0 | Fully active, able to carry on all pre-disease performance |
| | without restriction |
| 1 | Restricted in physically strenuous activity but ambulatory |
| | and able to carry out work of a light or sedentary nature, |
| | eg, light house work, office work |
| 2 | Ambulatory and capable of all selfcare but unable to carry |
| | out any work activities. Up and about more than 50% of waking |
| | hours |
| 3 | Capable of only limited selfcare, confined to bed or chair |
| | more than 50% of waking hours |
| 4 | Completely disabled. Cannot carry on any selfcare. Totally |
| | confined to bed or chair |
| 5 | Dead |

Oken M M, Creech R H, Tormey D C, et al. Toxicity and response criteria of the Eastern Cooperative Oncology Group. Am J Clin Oncol 1982; 5(6): 649-655.

APPENDIX 3
Sample List of Excluded Medications (subject to change)

| | | | Drugs Potentially Affected | | |
|---|---|---|---|---|---|
| | | | by Coadministration of | | |
| | Drugs Potentially Affecting | | Compound (10b) | | |
| | Exposure of Compound (10b) | | MATE1/2-K | P-gp | BCRP |
| Medication | CYP3A4 | P-gp | Substrates | Substrates | Substrates |
| Aliskiren | | | | X | |
| Alogliptin | | Inhibitor | | | |
| Ambrisentan | | | | X | |
| Amiodarone | | Inhibitor | | | |
| Amprenavir | Inhibitor | | | | |
| | (M) | | | | |
| Apalutamide | Inducer | Inducer | | | |
| | (S) | | | | |
| Apixaban | | | | X | |
| Apremilast | | | | X | |
| Aprepitant | Inhibitor | | | X | |
| | (M) | | | | |
| Asian ginseng (Panax ginseng) | | Inhibitor | | | |
| Asunaprevir | | Inhibitor | | | |
| Atazanavir | Inhibitor | | | X | |
| | (M) | | | | |
| Atazanavir/Ritonavir | Inhibitor | | | | |
| | (M) | | | | |
| Atorvastatin | | | | X | X |
| Avasimibe | Inducer | Inducer | | | |
| | (S) | | | | |
| Avatrombopag | | | | X | |
| Azithromycin | | Inhibitor | | | |
| Baricitinib | | | X | X | X |
| Betrixaban | | | | X | |
| Boceprevir | Inhibitor | | | X | |
| | (S) | | | | |
| Bosentan | Inducer | | | | |
| | (M) | | | | |
| Bosutinib | | | | X | |
| Budesonide | | | | X | |
| Canaglifozin | | Inhibitor | | X | |
| Capmatinib | | Inhibitor | | | |
| Captopril | | Inhibitor | | | |
| Carbamazepine | Inducer | Inducer | | | |
| | (S) | | | | |
| Carvedilol | | Inhibitor | | X | |
| Casopitant | Inhibitor | | | | |
| | (M) | | | | |
| Ceritinib | Inhibitor | | | | |
| | (S) | | | | |
| Cimetidine | Inhibitor | Inhibitor | | | |
| | (M) | | | | |
| Ciprofloxacin | Inhibitor | | | | |
| | (M) | | | | |
| Clarithromycin | Inhibitor | Inhibitor | | | |
| | (S) | | | | |
| Clobazam | | | | X | |
| Clopidogrel | | Inhibitor | | | |
| Cobicistat | Inhibitor | Inhibitor | | | |
| | (S) | | | | |
| Cobimetinib | | | | X | |
| Conivaptan | Inhibitor | Inhibitor | | X | |
| | (M) | | | | |
| Coumestrol | | | | | X |
| Cremophor EL | | Inhibitor | | | |
| Cremophor RH | | Inhibitor | | | |
| Crizotinib | Inhibitor | | | X | |
| | (M) | | | | |
| Cyclosporine | Inhibitor | | | X | |
| | (M) | | | | |
| Dabigatran etexilate | | | | X | |
| Dabrafenib | Inducer | | | X | |
| | (M) | | | | |
| Daclatasvir | | Inhibitor | | X | |
| Danoprevir/Ritonavir | Inhibitor | | | | |
| | (S) | | | | |
| Dan-shen (Salvia miltiorrhiza) | | Inducer | | | |
| Dantrolene | | | | | X |
| Darolutamide | | | | X | |
| Darunavir | Inhibitor | | | | |
| | (M) | | | | |
| Darunavir/Ritonavir | Inhibitor | | | | |
| | (M) | | | | |
| Digoxin | | | | X | |
| Diltiazem | Inhibitor | Inhibitor | | X | |
| | (M) | | | | |
| Diosmin | | Inhibitor | | | |
| Dolutegravir | | | | X | X |
| Doxorubicin | | | | X | |
| Dronedarone | Inhibitor | Inhibitor | | | |
| | (M) | | | | |
| Duvelisib | Inhibitor | | | X | X |
| | (M) | | | | |
| Edoxaban | | | | X | |
| Efavirenz | Inducer | Inducer | | | |
| | (M) | | | | |
| Elagolix | Inducer | Inhibitor | | | |
| | (M) | | | | |
| Elexacaftor | | | | X | |
| Eliglustat | | Inhibitor | | X | |
| Elvitegravir/Ritonavir | Inhibitor | | | | |
| | (S) | | | | |
| Enzalutamide | Inducer | | | | |
| | (S) | | | | |
| Erythromycin | Inhibitor | Inhibitor | | | |
| | (M) | | | | |
| Estrone sulfate | | | | | X |
| Etravirine | Inducer | | | | |
| | (M) | | | | |
| Everolimus | | | | X | |
| Faldaprevir | Inhibitor | | | | |
| | (M) | | | | |
| Fedratinib | Inhibitor | | | X | |
| | (M) | | | | |
| Felodipine | | Inhibitor | | | |
| Fexofenadine | | | | X | |
| Five-flavor berry | | Inhibitor | | | |
| Fidaxomicin | | | | X | |
| Fluconazole | Inhibitor | | | | |
| | (M) | | | | |
| Fluvoxamine | Inhibitor | Inhibitor | | | |
| | (M) | | | | |
| Fostamatinib | | Inhibitor | | | |
| Genistein | | Inducer | | | X |
| Gilteritinib | | | | X | |
| Ginkgo (Ginkgo biloba) | | Inhibitor | | | |
| Glasdegib | | | | X | X |
| Glecaprevir/Pibrentasvir | | Inhibitor | | X | X |
| Grapefruit juice | Inhibitor | | | | |
| | (S) | | | | |
| Grapefruit juice DS | Inhibitor | | | | |
| | (S) | | | | |
| Green tea | | Inducer | | | |
| Idelalisib | Inhibitor | | | X | |
| | (S) | | | | |
| Imatinib | Inhibitor | | | X | X |
| | (M) | | | | |
| Indacaterol | | | | X | |
| Indinavir | Inhibitor | | | X | X |
| | (S) | | | | |
| Indinavir/Ritonavir | Inhibitor | Inhibitor | | | |
| | (S) | | | | |
| Irinotecan | | | | X | X |
| Isavuconazole | Inhibitor | Inhibitor | | | |
| | (M) | | | | |
| Istradefylline | Inhibitor | Inhibitor | | | |
| | (M) | | | | |
| Itraconazole | Inhibitor | Inhibitor | | | |
| | (S) | | | | |
| Ivacaftor | | Inhibitor | | | |
| Ivosidenib | Inducer | | | X | |
| | (S) | | | | |
| Ketoconazole | Inhibitor | Inhibitor | | | |
| | (S) | | | | |
| Lapatinib | | Inhibitor | | X | |
| Larotrectinib | | | | X | X |
| Ledipasvir | | | | X | X |
| Lefamulin | Inhibitor | | | X | |
| | (M) | | | | |
| Lenvatinib | | | | X | X |
| Lersivirine | Inducer | | | | |
| | (M) | | | | |
| Lesinurad | Inducer | | | | |
| | (M) | | | | |
| Letermovir | Inhibitor | | | | |
| | (M) | | | | |
| Linagliptin | | | | X | |
| Loperamide | | | | X | |
| Lopinavir | Inducer | | | X | |
| | (M) | | | | |
| Lopinavir/Ritonavir | Inhibitor | Inhibitor | | | |
| | (S) | | | | |
| Lorlatinib | Inducer | | | | |
| | (M) | | | | |
| Lovastatin | | | | X | |
| Lumacaftor | Inducer | | | | |
| | (S) | | | | |
| Magnolia vine (Schisandra | Inhibitor | | | | |
| sphenanthera) | (M) | | | | |
| Metformin | | | X | | |
| Methotrexate | | | | | X |
| Methylprednisolone | | | | X | |
| Mibefradil | Inhibitor | Inhibitor | | | |
| | (S) | | | | |
| Mifepristone | Inhibitor | Inhibitor | | | |
| | (S) | | | | |
| Milk thistle (Silybum marianum) | | Inhibitor | | | |
| Mirabegron | | Inhibitor | | | |
| Mitotane | Inducer | | | | |
| | (S) | | | | |
| Modafinil | Inducer | | | | |
| | (M) | | | | |
| Nafcillin | Inducer | | | | |
| | (M) | | | | |
| Nefazodone | Inhibitor | Inhibitor | | | |
| | (S) | | | | |
| Nelfinavir | Inhibitor | Inhibitor | | X | |
| | (S) | | | | |
| Neratinib | | Inhibitor | | | |
| Netupitant | Inhibitor | | | | |
| | (M) | | | | |
| Nifedipine | | Inhibitor | | | |
| Nilotinib | Inhibitor | | | | |
| | (M) | | | | |
| Nintedanib | | | | X | |
| Nitrendipine | | Inhibitor | | | |
| Osimertinib | | Inhibitor | | | |
| Ozanimod | | | | | X |
| Paclitaxel | | | | X | |
| Panobinostat | | | | X | |
| Paritaprevir/Ritonavir/Ombitasvir | Inhibitor | Inhibitor | | | |
| | (S) | | | | |
| Paroxetine | | Inhibitor | | X | |
| Pexidartinib | Inducer | Inhibitor | | | |
| | (M) | | | | |
| Phenobarbital | Inducer | | | | |
| | (S) | | | | |
| Phenytoin | Inducer | Inducer | | | |
| | (S) | | | | |
| Piperine | | Inhibitor | | | |
| Pitavastatin | | | | X | X |
| Posaconazole | Inhibitor | | | X | |
| | (S) | | | | |
| Pralsetinib | | | | X | |
| Prazosin | | | | X | X |
| Primidone | Inducer | | | | |
| | (M) | | | | |
| Propafenone | | Inhibitor | | | |
| Quercetin | | Inhibitor | | | |
| Quinidine | | Inhibitor | | X | |
| Quinine | | Inhibitor | | | |
| Ranolazine | | Inhibitor | | | |
| Ravuconazole | Inhibitor | | | | |
| | (M) | | | | |
| Ribociclib | Inhibitor | | | | |
| | (S) | | | | |
| Rifabutin | Inducer | Inducer | | | |
| | (M) | | | | |
| Rifampin | Inducer | Inhibitor | | | |
| | (S) | | | | |
| Rifapentine | Inducer | | | | |
| | (S) | | | | |
| Rimegepant | | | | X | X |
| Riociguat | | | | X | X |
| Risperidone | | | | X | |
| Ritonavir | Inhibitor | Inhibitor | | X | |
| | (S) | | | | |
| Rivaroxaban | | | | X | |
| Rolapitant | | Inhibitor | | | |
| Romidepsin | | | | X | |
| Rosuvastatin | | | | | X |
| Rucaparib | | Inhibitor | | | |
| Saquinavir | Inhibitor | | | X | |
| | (S) | | | | |
| Saquinavir/Ritonavir | Inhibitor | Inhibitor | | | |
| | (S) | | | | |
| Sarecycline | | Inhibitor | | | |
| Selexipag | | | | X | |
| Semagacestat | Inducer | | | | |
| | (M) | | | | |
| Silodosin | | | | X | |
| Simeprevir | | Inhibitor | | | |
| Simvastatin | | | | X | |
| Sirolimus | | | | X | |
| Sofosbuvir | | | | X | X |
| Sofosbuvir/Velpatasvir/Voxilaprevir | | Inhibitor | | | |
| St. John's Wort Extract | Inducer | Inhibitor | | | |
| | (S) | | | | |
| Sulfasalazine | | | | | X |
| Suvorexant | | Inhibitor | | | |
| Tacrolimus | | | | X | |
| Talazoparib | | | | X | X |
| Talinolol | | Inhibitor | | X | |
| Talviraline | Inducer | | | | |
| | (M) | | | | |
| Telaprevir | Inhibitor | Inhibitor | | X | |
| | (S) | | | | |
| Telithromycin | Inhibitor | Inhibitor | | | |
| | (S) | | | | |
| Telmisartan | | Inhibitor | | | |
| Telotristat ethyl | Inducer | | | | |
| | (M) | | | | |
| Tenofovir alafenamide | | | | X | X |
| Tezacaftor | | | | X | |
| Thioridazine | Inducer | | | | |
| | (M) | | | | |
| Ticagrelor | | Inhibitor | | | |
| Tipranavir | | | | X | |
| Tipranavir/Ritonavir | Inhibitor | Inhibitor | | | |
| | (S) | | | | |
| Tivantinib | | Inducer | | | |
| Tofisopam | Inhibitor | | | | |
| | (M) | | | | |
| Tolvaptan | | Inhibitor | | | |
| Topotecan | | | | X | X |
| Troleandomycin | Inhibitor | | | | |
| | (S) | | | | |
| Tucatinib | Inhibitor | Inhibitor | | | |
| | (S) | | | | |
| Ubrogepant | | | | X | X |
| Umeclidinium | | | | X | |
| Valbenazine | | Inhibitor | | | |
| Valspodar (PSC 833) | | Inhibitor | | | |
| Vandetanib | | Inhibitor | | | |
| Velpatasvir | | Inhibitor | | X | X |
| Vemurafenib | | Inhibitor | | | |
| Venetoclax | | Inhibitor | | X | X |
| Verapamil | Inhibitor | Inhibitor | | X | |
| | (M) | | | | |
| Viekira Pak | Inhibitor | | | | |
| | (S) | | | | |
| Vinblastine | | | | X | |
| Vincristine | | | | X | |
| Voclosporin | | Inhibitor | | X | |
| Vorapaxar | | Inhibitor | | | |
| Voriconazole | Inhibitor | | | | |
| | (S) | | | | |
| Voxilaprevir | | | | X | X |
| Zanubrutinib | | Inhibitor | | | |

APPENDIX 4
List of Medications to be Used with Caution
OATP1B1 and/or OATP1B3 Substrates

Cholescystokinin
Eluxadoline
Estradiol-glucuronide
Fluvastatin
Glyburide
Nateglinide
Pitavastatin
Pravastatin
Repaglinide

### Example 8: Initial Results of a Phase 1/1B First-In-Human Study

The initial results were from Cohorts 1-5 of the Phase 1/1B First-in-Human Study of Example 6.

### Safety of Compound (10b)

Compound (10b) was well tolerated in Cohorts 1-5, where compound (10b) was dosed at 80 mg, 150 mg, 250 mg, 400 mg, and 550 mg once daily, respectively. No safety findings impacting the risk or benefit of compound (10b) reported in the 19 patients dosed. Nine (9) treatment-emergent serious adverse events (SAEs) were reported in 7 patients, none of which were deemed related to the treatment with compound (10b). No adverse events (AEs) led to study discontinuation and no dose-limiting toxicity (DLTs) occurred. Notable treatment-emergent adverse events (TEAEs) are listed in Table 6.

TABLE 6
Notable treatment-emergent adverse events (TEAEs)

| | Number of | | | | | | | | |
|---|---|---|---|---|---|---|---|---|---|
| | patients | | | | | | | | |
| TEAEs | (n = 19) | Grades | | | Cohorts | | | | |
| Edema | 4 | ✓ | ✓✓ | | ✓ | ✓ | | ✓ | ✓ |
| LFT increases | 2 | | ✓ | ✓ | | ✓ | | ✓ | |
| LVEF decrease | 1 | | ✓ | | | | | | |
| Rash | 2 | ✓ | ✓ | | | | | ✓ | ✓ |
| Nausea/ | 4 | ✓✓ | ✓✓ | | | ✓ | | ✓✓ | ✓ |
| vomiting | | | | | | | | | |
| Diarrhea | 5 | ✓ × | ✓ | | | ✓ | ✓ | ✓✓ | ✓ |
| | | 4 | | | | | | | |
| Constipation | 1 | ✓ | | | | ✓ | | | |

### Pharmacokinetic (PK) Profiles of Compound (10b)

Compound (10b) was found to have a continued increase of pharmacokinetic exposure in Cohort 5, where compound (10b) was dosed at 550 mg once daily. Data are presented in Table 7A and Table 7B.

TABLE 7A
PK Exposure of Compound (10b) in Cohorts
1-5 (C1D1 - Day 1 of Cycle 1)

| Dose | | Cmax | Tmax | t_{1/2} | AUClast | | |
|---|---|---|---|---|---|---|---|
| (mg) | N | (ng/mL) | (h) | (h) | (ng · h/mL) | Cmax/D | AUC/D |
| 80 | 1 | 405 | 2.00 | (14.9) | 4890 | 5.06 | 61.1 |
| 150 | 3 | 651 | 2.00 | (15.6) | 9647 | 4.34 | 64.3 |
| 250 | 2* | 797 | 3.00 | 9.44^{a} | 10605 | 3.19 | 42.4 |
| 400 | 5 | 1538 | 3.00 | 10.1^{a} | 18780 | 3.85 | 47.0 |
| 550 | 6 | 2548 | 2.67 | (13.5) | 37700 | 4.63 | 68.6 |

AUC/D calculated using AUClast;
t_{1/2} values in parentheses should be interpreted with caution (associated with large extrapolated AUC);
*1 subject with active gastritis (due to H. pylori) during PK visit exhibited outlying PK values and was excluded from mean calculation; and
^{a}N = 1

TABLE 7B
PK Exposure of Compound (10b) in Cohorts
1-5 (C2D1 - Day 1 of Cycle 2)

| Dose | | Cmax | Tmax | t_{1/2} | AUClast | P/T | AR | AR |
|---|---|---|---|---|---|---|---|---|
| (mg) | N | (ng/mL) | (h) | (h) | (ng · h/mL) | Ratio | Cₘₐₓ | AUC |
| 80 | 1 | 622 | 1.00 | 10.0 | 6290 | 6.82 | 1.54 | 1.29 |
| 150 | 3 | 1119 | 3.33 | 10.1^{a} | 18200 | 3.19 | 1.70 | 1.90 |
| 250 | 1 | 1380 | 2.00 | NR | 21600 | 2.81 | 1.82 | 1.85 |
| 400 | 3 | 2267 | 3.00 | (12.3)^{a} | 32000 | 3.42 | 1.54 | 1.82 |

t_{1/2} values in parentheses should be interpreted with caution (associated with large extrapolated AUC); and
^{a}N = 1

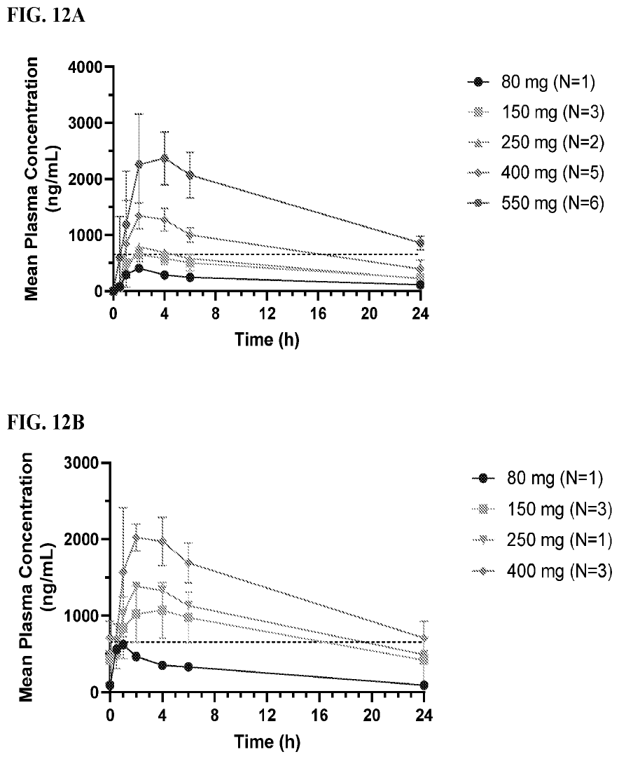

FIGS. 12A-12B show mean plasma concentrations of compound (10b) in Cohorts 1-5 of Example 6. FIG. 12A: Day 1 of Cycle 1 (C1D1); and FIG. 12B: Day 1 of Cycle 2 (C2D1). Dotted line represents in vivo IC₅₀ (1.5 μM; the KYSE-520 xexograft DUXP6 IC₅₀ is 656 ng/ml) and predicted efficacious Cₘₐₓ (5.3 μM).

pERK Inhibition of Compound (10b)

Compound (10b) was found to have deeper PBMC pERK inhibition in cohort 5 (550 mg): ˜ 95% of the inhibition was observed 4 hours post dose and the inhibition was sustained 24 hours post dosing.

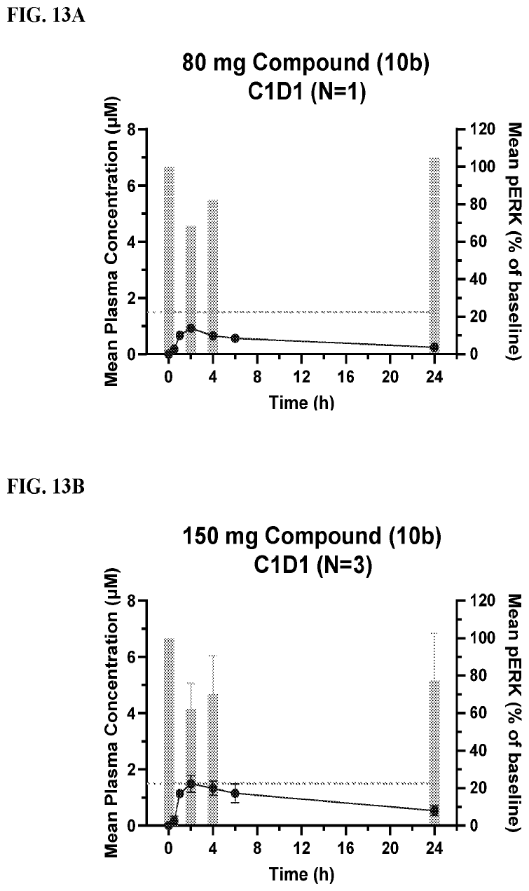

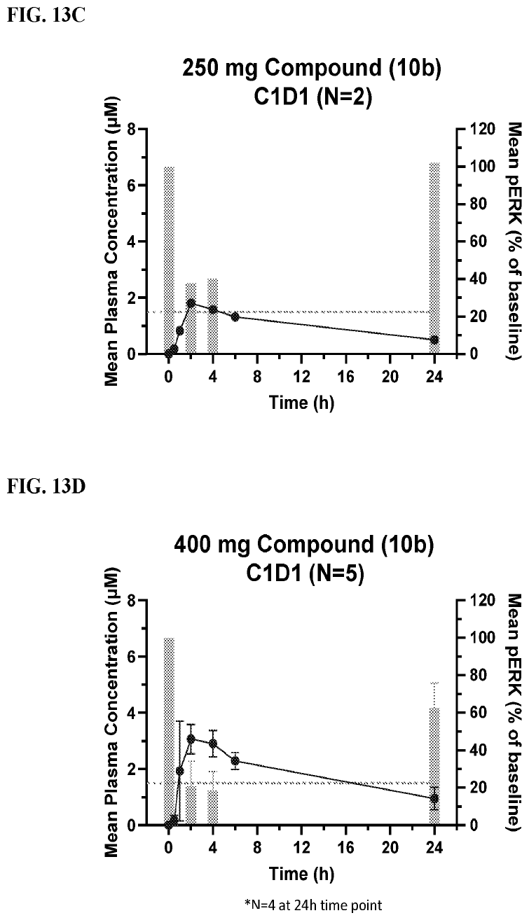

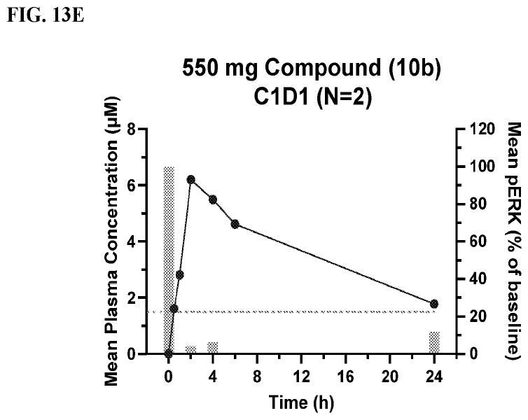

FIGS. 13A-13E show pERK inhibition of compound (10b) in Cohorts 1-5 of Example 6. FIG. 13A: Cohort 1; FIG. 13B: Cohort 2: FIG. 13C: Cohort 3; FIG. 13D: Cohort 4; and FIG. 13E: Cohort 5. Dotted line represents IC₅₀ of 1.5 μM. Bars represent PD (% pERK) and filled circles represent PK.

### SUMMARY

In summary, the clinical PK/PD profile of compound (10b) is consistent with nonclinical data. Compound (10b) is compared to other SHP2 inhibitors (e.g., TNO-155 and RMC-4630), as shown in Table 8.

TABLE 8
Comparison of Compound (10b) to Other SHP2 Inhibitors

| Properties | TNO-155 | RMC-4630 | Compound (10b) |
|---|---|---|---|
| PD target defined at | >50% DUSP6 mRNA | >50% pERKinhibition | >50% DUSP6 mRNA |
| maximal nonclinical | inhibition for majority | for dosing interval | inhibition for majority |
| efficacy | of dosing interval | | of dosing interval |
| Human PD | >50% DUSP6 mRNA | >30% pERKinhibition | 95% @ 4 h and 90% |
| | inhibition in tumors | in half of tumors | @ 24 h |
| | at ≥20 mg daily | analyzed at ≥20 mg | pERKinhibition in |
| | | daily | PBMC at 550 mg |
| | | | dose level |
| Human DLTs | LVEF decrease, liver | AEs lead to dose | None |
| | enzyme elevation, | interruption in 50% | |
| | diarrhea | patients; diarrhea, | |
| | | thrombocytopenia, | |
| | | edema are most | |
| | | common AEs | |
| Human Half life | 35 hr | 28-33 hr | 10-16 hr |
| Dosing schedule | No RP2D determined; | Intermittent 200 mg | Tolerable continuous |
| | 95% of patients had 1 | D1D2qw dosing to | daily dosing cohorts |
| | week of dosing | improve tolerability | 1-5 |
| | holiday (2/3 weeks | | |
| | on, 1 week off) | | |

The profile and dosing schedule of compound (10b) can provide dosing flexibility for combinations while providing an excellent target coverage at tolerable doses.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method of treating cancer, comprising administering to a subject in need thereof a therapeutically effective amount of a compound represented by formula (I): 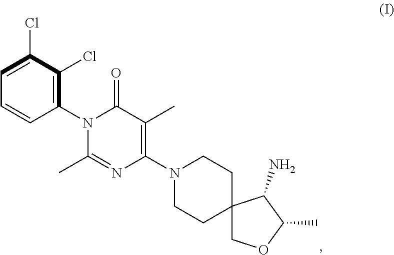
or a pharmaceutically acceptable salt, hydrate, solvate, stereoisomer, conformational isomer, tautomer, or a combination thereof, wherein the subject has (i) one or more mutations in MAPK pathway, provided that the one or more mutations in MAPK pathway are other than a BRAF mutation comprising V600X mutation, and/or (ii) one or more mutations in PTPN11.

2. (canceled)

3. The method of claim 1, wherein the compound of formula (I) is represented by formula (10b): 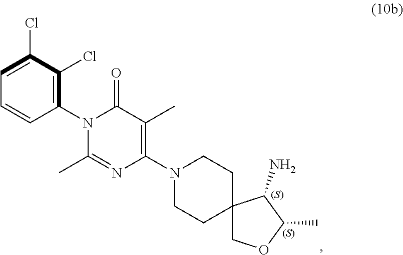
having the name of 6-((3S,4S)-4-amino-3-methyl-2-oxa-8-azaspiro[4.5]decan-8-yl)-3-(Rₐ)-(2,3-dichlorophenyl)-2,5-dimethylpyrimidin-4 (3H)-one.

4. (canceled)

5. (canceled)

6. The method of claim 1, wherein the cancer is an advanced or metastatic KRAS G12C-positive non-small cell lung cancer (NSCLC).

7. (canceled)

8. The method of claim 1, wherein the cancer is an advanced or metastatic KRAS mutant solid tumor.

9. The method of claim 1, wherein the subject has one or more mutations in MAPK pathway selected from the group consisting of one or more mutations in KRAS, NRAS, HRAS, CRAF, BRAF, NRAF, MAPK/ERK, MAPKK/MEK, NF1, IGFR, EGFR, PDGFR, VEGFR, FGFR, CCKR, NGFR, EphR, AXLR, KEAP1, TIE receptor, RYK receptor, DDR receptor, RET receptor, ROS receptor, LTK receptor, ROR receptor, MuSK receptor, and a combination thereof.

10. The method of claim 1, wherein the cancer is an advanced or metastatic neurofibromatosis type 1 (NF1) loss of function (LOF) solid tumor, an advanced or metastatic EGFR-positive solid tumor, an advanced or metastatic BRAF class II/III mutant solid tumor, chordoma, or notochordal sarcoma.

11. (canceled)

12. (canceled)

13. The method of claim 1, wherein the subject has an EGFR mutation comprising an EGFR exon 19 deletion, exon 20 insertion, L858X mutation, T790X mutation, C797X mutation, G719X mutation, L861X mutation, S768X mutation, E709X mutation, or any combination thereof.

14-18. (canceled)

19. The method of claim 1, wherein the subject does not have one or more additional activating mutations in PTPN11 (SHP2), MEK, or RAS Q61.

20-24. (canceled)

25. The method of claim 1, wherein the therapeutically effective amount is a total daily dosage of from about 10 mg to about 2000 mg, from about 50 mg to about 2000 mg, from about 80 mg to about 2000 mg, from about 80 mg to about 1000 mg, from about 80 mg to about 700 mg, from about 80 mg to about 550 mg, from about 80 mg to about 450 mg, from about 80 mg to about 400 mg, from about 80 mg to about 250 mg, or from about 80 mg to about 150 mg of the compound of formula (I) or (10b), on a salt-free and anhydrous basis.

26. (canceled)

27. The method of claim 1, wherein the therapeutically effective amount is a total daily dosage of about 80 mg, about 150 mg, about 250 mg, about 400 mg, about 450 mg, about 550 mg, or about 700 mg of the compound of formula (I) or (10b), on a salt-free and anhydrous basis.

28-34. (canceled)

35. The method of claim 1, wherein the compound of formula (I) or (10b) is administered orally.

36. (canceled)

37. The method of claim 1, wherein the compound of formula (I) or (10b) is administered once daily.

38. The method of claim 1, wherein the compound of formula (I) or (10b) is administered to the subject without food, at least about 8 hours prior to the administration and at least about 2 hours post the administration.

39. (canceled)

40. (canceled)

41. The method of claim 1, wherein administration of the therapeutically effective amount of formula (I) or (10b) reduces a volume of and/or stabilizes the cancer for at least about one month.

42-45. (canceled)

46. A method of treating a tumor, comprising administering to a subject in need thereof a therapeutically effective amount of a compound represented by formula (10b): 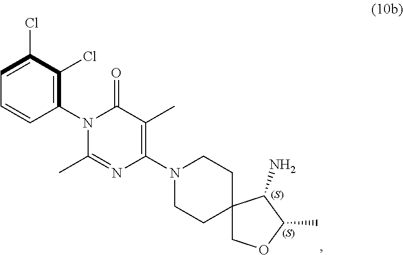
or a tautomer thereof, wherein the subject has (i) one or more mutations in MAPK pathway, provided that the one or more mutations in MAPK pathway are other than a BRAF mutation comprising V600X mutation, and/or (ii) one or more mutations in PTPN11.

47. The method of claim 46, wherein the tumor is an advanced or metastatic KRAS G12C-positive non-small cell lung cancer (NSCLC), an advanced or metastatic EGFR-positive NSCLC, an advanced or metastatic KRAS mutant solid tumor, an advanced or metastatic neurofibromatosis type 1 (NF1) loss of function (LOF) solid tumor, an advanced or metastatic BRAF class II/III mutant solid tumor, chordoma, or notochordal sarcoma.

48-53. (canceled)

54. The method of claim 46, wherein the therapeutically effective amount is a total daily dosage of about 450 mg of the compound of formula (10b), on a salt-free and anhydrous basis.

55. The method of claim 46, wherein the compound of formula (10b) is administered once daily.

56. The method of claim 1, wherein the compound of formula (10b) is administered orally.

57. A kit for treating cancer or a solid tumor in a subject, comprising:
a therapeutically effective amount of a compound represented by formula (I): 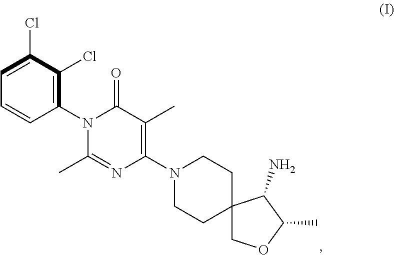
or a pharmaceutically acceptable salt, hydrate, solvate, stereoisomer, conformational isomer, tautomer, or a combination thereof; or
a compound represented by formula (10b): 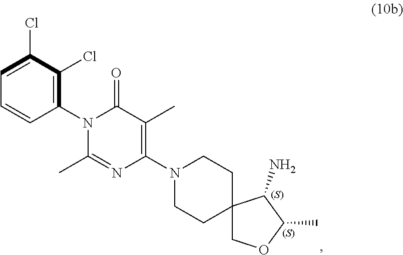
or a tautomer thereof,
together with instruction for effective administration, wherein the subject has (i) one or more mutations in MAPK pathway, provided that the one or more mutations in MAPK pathway are other than a BRAF mutation comprising V600X mutation, and/or (ii) one or more mutations in PTPN11.
